(19) United States
(12) Patent Application Publication
(10) Pub. No.: US 2025/0290151 A1
(43) Pub. Date: Sep. 18, 2025

(54) Differentially-Methylated Regions of the Genome Useful as Markers of Embryo-Adult Transitions

(71) Applicants: Michael D. West, Mill Valley, CA (US); Karen Chapman, Mill Valley, CA (US)

(72) Inventors: Michael D. West, Mill Valley, CA (US); Karen Chapman, Mill Valley, CA (US)

(21) Appl. No.: 18/926,271

(22) Filed: Oct. 24, 2024

Related U.S. Application Data
(63) Continuation of application No. 17/251,145, filed on Dec. 10, 2020, now abandoned.
(60) Provisional application No. 62/891,225, filed on Aug. 23, 2019.

(51) Int. Cl. C12Q 1/6886; C12Q 1/6806

(52) U.S. Cl. CPC C12Q 1/6886; C12Q 1/6806; C12Q 2600/118; C12Q 2600/154

(57) ABSTRACT

The present invention relates to compositions and methods for the assay, diagnosis, prognosis or monitoring of the embryonic, fetal, and adult epigenetic states of a human genome. The disclosed methods are useful in monitoring the progress of in vitro and in vivo cellular reprogramming and the diagnosis, prognosis or monitoring of cancer in an individual. Specifically, the invention provides methods for the detection and interpretation of observed differential DNA methylation patterns and associated epigenetic modifications to core histones in determining the developmental status of human cells for the detection and characterization of cancer cells and determining optimum therapeutic modalities.

### CROSS REFERENCE TO RELATED APPLICATIONS

This application claims priority benefit of the filing date of U.S. Provisional Patent Application 62/891,225, filed Aug. 23, 2019, the content of which is incorporated herein by reference in their entirety.

### FIELD OF THE INVENTION

The present invention relates to compositions and methods for the assay, diagnosis, prognosis, monitoring and modulation of the embryonic, fetal, and adult epigenetic states of a human genome. The disclosed methods are useful in monitoring the progress of in vitro and in vivo cellular reprogramming and the diagnosis, prognosis and/or monitoring of cancer and the determination of optimum therapeutic regimens for the treatment of cancer in an individual. Specifically, the invention provides methods for the detection and interpretation of observed differential DNA methylation patterns and/or associated epigenetic modifications to core histones in determining the developmental status of human cells useful in quality control assays and choice of therapeutic modalities.

### BACKGROUND

Advances in stem cell technology, such as the isolation and propagation in vitro of human pluripotent stem (hPS), including but not limited to human embryonic stem (hES) and induced pluripotent stem (iPS) cells, constitute an important new area of medical research. hPS cells have a demonstrated potential to be propagated in the undifferentiated state and then to be induced subsequently to differentiate into any and all of the cell types in the human body, including cells displaying markers of early pre-fetal, and prenatal development (see PCT application Ser. No. PCT/US2006/013519 filed on Apr. 11, 2006 and titled “Novel Uses of Cells With Prenatal Patterns of Gene Expression”; U.S. patent application Ser. No. 11/604,047 filed on Nov. 21, 2006 and titled “Methods to Accelerate the Isolation of Novel Cell Strains from Pluripotent Stem Cells and Cells Obtained Thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, each incorporated in their entirety herein by reference).

While closely matching the transcriptional profile of normal hES cells, hiPS cells have subtle differences including frequently not reprogramming telomere length (Vaziri et al 2010, Spontaneous reversal of the developmental aging of normal human cells following transcriptional reprogramming Regen Med 5(3):345-363), as well as epigenetic abnormalities such as displaying an epigenetic memory of the cells from which they were derived (in other words, a lack of complete epigenetic reprogramming). We previously disclosed methods to assay the telomere length of reprogrammed cells as a quality control step in manufacturing (West, M.D., Methods For Telomere Length And Genomic DNA Quality Control Analysis In Pluripotent Stem Cells, U.S. Patent Application 20170335392) incorporated herein by reference. Nevertheless, there remains a need for additional markers that provide improved sensitivity for quantifying the extent of reprogramming of somatic cells to pluripotency (iPS cells) and partial in vivo reprogramming to reverse aging and induce tissue regeneration (iTR). More specifically, there is a need for improved methods to determine the extent of reprogramming the epigenome during in vitro reprogramming of somatic cells to pluripotency (iPS cells) and partial in vivo reprogramming to reverse aging and induce tissue regeneration (iTR).

We previously disclosed gene expression markers as well as regulators of the embryonic-fetal transition (EFT) as well as the neonatal transition (NT) described in “Compositions and Methods for Induced Tissue Regeneration in Mammalian Species” (international patent application publication number WO 2014/197421), incorporated herein by reference in its entirety and “Improved Methods for Detecting and Modulating the Embryonic-Fetal Transition in Mammalian Species” (international patent application publication number WO 2017/214342, incorporated herein by reference in its entirety); and “Induced Tissue Regeneration Using Extracellular Vesicles” (U.S. Provisional Patent Applications 62/872,246, filed Jul. 9, 2019, and 62/825,732, filed Mar. 28, 2019), each incorporated herein by reference in their entirety. We taught that these genes associated with the developmental transition from embryonic development to fetal and later adult development were also responsible for the transition from the regenerative state observed in embryonic (pre-fetal tissues) such as the ability of those tissue to undergo scarless wound repair, to the later state of scarring in lieu of regeneration observed in fetal and adult tissues. We also described a subset of specific genes whose pattern of expression on an mRNA level in the embryonic (pre-EFT) state matches the expression in the majority of all human cancers (i.e. they are pan-cancer markers).

Alterations in gene expression during development, aging, and carcinogenesis have been associated with altered DNA methylation, including, but not limited to, altered DNA methylation in CpG islands. In the case of cancer, methylation of tumor suppressor genes has been implicated in carcinogenesis (Kanai Y., Hirohashi S., “Alterations of DNA methylation associated with abnormalities of DNA methyltransferases in human cancers during transition from a precancerous to a malignant state,” Carcinogenesis, 2007, 28, 2434-2442). Importantly, whole genome bisulfite sequencing (WGBS) of the genome of diverse types of cancer when compared to normal tissue counterparts have revealed a large number of differentially-methylated regions (DMRs) in the cancer cell genome such as those associated with CpG sequences (Su J. Huang Y H, Cui X, et al. Homeobox oncogene activation by pan-cancer DNA hypermethylation, Genome Biol. 2018; 19(1):108. Published 2018 Aug. 10. doi:10.1186/si3059-018-1492-3) (referred to as Su et al, 2018 herein).

Since the methylation status of DNA is relatively stable in most biological settings, such as in blood, there is great interest in detecting cell-free DNA (cfDNA) in blood derived from tumors (circulating tumor-derived DNA (ctDNA) using differential methylation as a marker. While methods of detecting differentially-methylated DNA sequences in the blood and other body fluids are well known in the art, novel and defined differentially methylated regions such as those that precisely identify cells displaying a phenotype of a cell before versus after the EFT and the neonatal transition (NT) are needed that are capable of being used for detecting rare cells or circulating DNA such as that originating from cancers in body fluids (liquid biopsies) that have reverted to said embryonic as opposed to fetal/adult pattern of gene expression (embryo-onco phenotype), or monitoring the progress of in vitro or in vivo reprogramming. In addition, the present invention discloses the novel observation that cells within tumors are heterogeneous in regard to pre-EFT or post-EFT maturation status, and the population of cells surviving commonly-used chemotherapeutic or radiotherapeutic regimens (commonly designated cancer stem cells (CSCs)) are not undifferentiated stem cells, but actually show a post-EFT phenotype that result in slower growth and relative resistance to apoptosis. Therefore, the methods and compositions of the present invention provide means of assaying the state of maturation of cancer cells as to whether they are adult-like cancer (AC) cells or dematured cancer (DC) cells, which in turn, are useful in the diagnosis and prognosis of cancer and determining optimum therapeutic choices targeting and ablating AC or DC cells.

### SUMMARY OF THE INVENTION

The present invention teaches novel compositions and methods related to the detection of differentially methylated regions (DMRs) of DNA associated with the EFT. More specifically, the present invention relates to novel composition and methods related to DMRs that are hypermethylated in normal cells in a pre-fetal state of maturation. Said pre-fetal cells with the hypermethylated DMRs of the present invention may be fully differentiated and yet not fully mature in that they display a phenotype differing substantially from corresponding cells in the post-EFT state including increased sensitivity to apoptosis, increased regenerative and proliferative potential, and increased potential for senolysis in the pre-fetal (pre-EFT) state.

The present invention discloses the maturation of cells at the EFT, while not necessarily altering their differentiated state, nevertheless acts as a tumor suppression, anti-regeneration, and antiviral mechanism. Therefore, the DMRs of the present invention provide methods to assay the extent of reprogramming of normal adult somatic cell types back to an embryonic or regenerative pattern of gene expression, to assay the metabolic state of cells such whether the cells have shifted toward glycolytic or oxidative phosphorylation as a major energy source, and determine the associated epigenetic state of said cells. These varied aspects of the pre-fetal phenotype are also referred to herein as the “embryo-onco phenotype.” The present invention discloses that these DMR markers are unexpectedly nearly universal hallmarks of diverse types of malignancies, including diverse sarcomas, carcinomas, and adenocarcinomas (i.e. are “pan-cancer markers.”)

In addition, the present invention teaches that an important feature of the heterogeneity of cancer cells in a tumor is the maturation status of the cancer cell. The present invention teaches that cancer cells can alternate their developmental status from dematured (pre-EFT) cancer cells (DC cells) to adult-like AC cells and from adult-like AC cells to DC cells. Unlike the cancer stem cell model which predicts that the subset of cancer cells surviving chemotherapy or radiation therapy are developmentally less-differentiated “stem cells” perhaps even expressing pluripotency markers such as OCT4, KLF4, SOX2, and MYC or other ES-specific transcription factors, the AC/DC model of developmental heterogeneity discloses that the heterogeneity of cancer cells is the state of maturation only in regard to being pre-EFT or post-EFT in phenotype. Furthermore the present invention discloses that the residual cancer cells following chemotherapy or radiation therapy are enriched in AC cells that are more mature, and more resistant to apoptosis (FIG. 1) as opposed to the currently widespread belief that the residual cells are more undifferentiated cancer stem cells.

The identification of novel methylated/demethylated genomic DNA that provides markers of the EFT therefore could therefore allow protocols for the amplification and detection of markers of EFT in tumors useful in diagnostic, prognostic, and therapeutic decision-making as well as in detecting the presence of cancer in a patient by detecting circulating tumor DNA (ctDNA) in blood, bronchial lavage, urine, or other body fluids or tissues using downstream detection methods of differentially-methylated DNA known in the art. Novel DMRs described in the present invention provide the novel assay of the embryonic (pre-fetal) as well as fetal (prenatal) markers useful in identifying malignant, and in some cases pre-malignant cells, that have reverted to said embryonic (pre-fetal) phenotype for the purpose of diagnosis and therapy, and for making clinical decisions relating to the advisability of maturing those cells to a more mature fetal or adult phenotype (also referred herein as “induced Cancer maturation” or “iCM”) to arrest their growth and/or metastasis, or to induce the embryonic (pre-fetal) phenotype in cancer stem cells to increase their susceptibility to apoptosis in response to chemotherapeutic regimens. This latter technology of reverting CSCs to a more primitive embryonic state is counterintuitive. The present invention shows that by causing iTR in cancer stem cells (referred to herein as “induced Senolysis of Cancer Stem Cells” or “iS-CSC”), the result is the production of cells with an embryonic phenotype (pre-fetal) pattern of gene expression less resistant to apoptosis. Therefore the present invention provides methods to detect and target malignant cells that have adult pattern of gene expression as well as providing methods to screen for agents capable of causing iS-CSC. Surprisingly, such diagnosis relates to a broad array of cancer types including carcinomas, adenocarcinomas, and sarcomas.

Embodiments of the disclosure are directed to methods of determining the developmental staging of cells that were the source of a sample of human DNA. More specifically, the present invention provides compositions and methods for determining whether human DNA contains methylated or unmethylated CpG epigenetic marks of embryonic (pre-fetal), fetal (prenatal), or postnatal (adult) marks. Said modifications unexpectedly provide useful broad pan-cancer markers for the diagnosis, prognosis and treatment of cancer as well as markers of the completeness of the in vitro transcriptional reprogramming of cells to pluripotency (iPS cell reprogramming) or the in vivo reprogramming of cells and tissues to reverse aging or to induce tissue regeneration (iTR) in diverse tissues in the body.

The disclosed methods are pan-cancer in nature and may therefore be used for diagnosing and/or treating an unexpectedly broad array of cancer types including but not limited to: carcinomas and adenocarcinomas (including but not limited to of any type, including solid tumors and leukemias including: apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, merkel cell, mucinous, non-small cell lung, oat cell, papillary, scirrhous, bronchiolar, bronchogenic, squamous cell, and transitional-cell), histiocytic disorders, leukemia (e.g., b-cell, mixed-cell, null-cell, T-cell, T-cell chronic, HTLV-II-associated, lymphocytic acute, lymphocytic chronic, mast-cell, and myeloid), histiocytosis malignant, Hodgkin's disease, immunoproliferative small, non-Hodgkin's lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, leydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, osteosarcoma, rhabdomyosarcoma, sarcoma (e.g., Ewing's, experimental, Kaposi's, and mast-cell), neoplasms of the bone, breast, digestive system, colorectal, liver, pancreatic, pituitary, testicular, orbital, head and neck, central nervous system, acoustic, pelvic, respiratory tract, and urogenital systems, neurofibromatosis, cervix dysplasia, hepatocellular carcinomas, epidermoid carcinomas, renal cell adenocarcinomas, colorectal carcinomas and adenocarcinomas, esophageal and head and neck cancers, bronchio-alveolar carcinomas such as non-small cell lung cancer, small cell lung cancer, mammary gland carcinomas, mammary ductal carcinomas; gastric carcinomas, prostate carcinomas, uterine adenocarcinomas, embryonal neuroectodermal tumors and teratocarcinomas, brain cell cancers such as glioblastomas and neuroblastomas, blood cell cancers such as histiocytic and lymphoblastic lymphomas and B-cell lymphoblastic leukemia, or sarcomas (including but not limited to embryonic and alveolar rhabdomyosarcomas, osteosarcomas, chondrosarcomas, liposarcomas, giant cell sarcomas of bone, uterine sarcomas, leiomyosarcomas, Wilms tumor, Ewing's sarcoma, pagetoid sarcoma, epithelioid sarcoma, synovial sarcomas, fibrosarcomas, and spindle cell sarcomas).

In addition, the disclosed methods may be used for staging the developmental status of an unexpectedly broad array of human somatic cell types including but not limited to: derivatives of the three germ layers endoderm, mesoderm, and ectoderm including neural crest, examples of endodermal somatic cell types being, but not limited to esophageal, tracheal, lung, gastrointestinal, liver, and pancreatic cells. Examples of mesodermal somatic cell types being, but not limited to bone, cartilage, tendon, skeletal, cardiac, and smooth muscle, renal, dermal, white and brown adipose, blood, and vascular endothelial cells. Examples of ectodermal somatic cell types being, but not limited to CNS and PNS neuronal cells including but not limited to neurons, glial and sensory neuronal cells such as those in the retina and inner ear. Examples of neural crest somatic cell types being, but not limited to connective tissues of the head and neck including dermal, cartilage, bone, meningeal, and adrenal cortical cells. Said staging is useful in assaying the completeness of the in vitro transcriptional reprogramming of cells to pluripotency (iPS cell reprogramming) or the in vivo reprogramming of cells and tissues to reverse aging or to induce tissue regeneration (iTR) in diverse tissues in the body.

In one embodiment, the method comprises steps to identify DMRs useful in distinguishing embryonic (pre-fetal) stage cells from postnatal stage cells, said method comprised of the steps: 1) determining the methylation status of the CpGs in the DNA of pluripotent stem cell-derived progenitor cells and their adult cell counterparts, 2) comparing the methylation of the embryonic (pre-fetal) cells to their post-natal counterparts to identify statistically-significant DMRs.

In another embodiment, the method comprises steps to diagnose cancer being: 1) obtaining DNA from biopsied human tissue or body fluid-derived cell-free DNA (cfDNA), 2) measuring the levels of methylated or unmethylated DNA within DMRs of the present invention, 3) determining whether the % methylation of the CpGs in the DMR or multiple DMRs is statistically-significant higher levels than normal tissue or cfDNA, 4) diagnosing cancer based on statistically-significant higher methylation in the DMRs of the sample compared to a normal control sample.

In another embodiment, the method comprises steps to diagnose cancer being: 1) obtaining DNA from biopsied human tissue or body fluid-derived cell-free DNA (cfDNA), 2) converting unmethylated cytosine residues to uracil using bisulfite, 3) sequencing the DMRs of the present invention to determine whether the % methylation of the CpGs in the DMR or multiple DMRs is statistically-significant higher levels than normal tissue or ccfDNA, 4) diagnosing cancer based on statistically-significant higher methylation in the DMRs of the sample compared to a normal control sample.

In another embodiment, the method comprises steps to diagnose cancer being: 1) obtaining DNA from biopsied human tissue or body fluid-derived cell-free DNA (cfDNA), 2) converting unmethylated cytosine residues to uracil using bisulfite, 3) digestion of DNA sample with methylation-specific restriction enzymes, 4) PCR amplifying sequences within the DMR region to determine whether the % methylation of the CpGs in the DMR or multiple DMRs is statistically-significant higher levels than normal tissue or cfDNA, 4) diagnosing cancer based on statistically-significant higher methylation in the DMRs of the sample compared to a normal control sample.

In another embodiment, the method comprises steps to diagnose cancer being: 1) obtaining DNA from biopsied human tissue or body fluid-derived cell-free DNA (cfDNA), 2) measuring the levels of methylated or unmethylated DNA within DMRs of the present invention, 3) determining whether the % methylation of the CpGs in the DMR or multiple DMRs is statistically-significant higher levels than normal tissue or cfDNA, 4) diagnosing cancer based on statistically-significant higher methylation in the DMRs of the sample compared to a normal control sample.

In another embodiment, the method comprises steps to diagnose cancer being: 1) obtaining DNA from biopsied human tissue or body fluid-derived cell-free DNA (cfDNA), 2) converting unmethylated cytosine residues to uracil using bisulfite, 3) sequencing the DMRs of the present invention to determine whether the % methylation of the CpGs in the DMR or multiple DMRs is statistically-significant higher levels than normal tissue or cfDNA, 4) diagnosing cancer based on statistically-significant higher methylation in the DMRs of the sample compared to a normal control sample.

In another embodiment, the method comprises steps to diagnose cancer being: 1) obtaining DNA from biopsied human tissue or body fluid-derived cell-free DNA (cfDNA), 2) converting unmethylated cytosine residues to uracil using bisulfite, 3) digestion of DNA sample with methylation-specific restriction enzymes, 4) PCR amplifying sequences within the DMR region to determine whether the % methylation of the CpGs in the DMR or multiple DMRs is statistically-significant higher levels than normal tissue or cfDNA, 4) diagnosing cancer based on statistically-significant higher methylation in the DMRs of the sample compared to a normal control sample.

In another embodiment, the method comprises steps to diagnose cancer being: 1) obtaining DNA from body fluid-derived cell-free DNA (cfDNA), 2) removal of the nucleosomes containing fetal or adult-specific histone epigenetic modifications H3K4me1, H3K4me2, H3K4me3, H3K9Ac, and H2AZ using affinity separation methods, 3) converting unmethylated cytosine residues to uracil using metabisulfite, 4) PCR amplifying sequences within the DMR region to determine whether the % methylation of the CpGs in the DMR or multiple DMRs is statistically-significantly higher levels than normal tissue or cfDNA, 5) diagnosing cancer based on statistically-significant higher methylation in the DMRs of the sample compared to a normal control sample.

In another embodiment, the method comprises steps to diagnose cancer being: 1) obtaining DNA from body fluid-derived cell-free DNA (cfDNA), 2) isolation of the nucleosomes containing histone epigenetic modifications present in the DMR regions of the present invention including H3K9me2 and H3K9me3 using affinity separation methods, 3) converting unmethylated cytosine residues to uracil using metabisulfite, 4) PCR amplifying sequences within the DMR region to determine whether the % methylation of the CpGs in the DMR or multiple DMRs is statistically-significantly higher levels than normal tissue or cfDNA, 5) diagnosing cancer based on statistically-significant higher methylation in the DMRs of the sample compared to a normal control sample.

In another embodiment, the method comprises steps to detect cancer stem cells (CSCs) that would respond to iS-CSC or alternatively iCM being: 1) obtaining DNA from biopsied human tissue, 2) measuring the levels of methylated or unmethylated DNA within DMRs of the present invention, 3) determining whether the % methylation of the CpGs in the DMR or multiple DMRs is statistically-significant higher levels than normal tissue, 4) diagnosing therapy-resistant CSCs based on statistically-significantly lower methylation in the DMRs of the sample compared to a therapy-responsive sample.

In another embodiment, the method comprises steps to detect cancer stem cells (CSCs) that would respond to iS-CSC or alternatively iCM being: 1) obtaining DNA from biopsied human tissue, 2) converting unmethylated cytosine residues to uracil using bisulfite, 3) sequencing the DMRs of the present invention to determine whether the % methylation of the CpGs in the DMR or multiple DMRs is statistically-significant higher levels than normal tissue or Cf-DNA, 4) diagnosing therapy-resistant CSCs based on statistically-significantly lower methylation in the DMRs of the sample compared to a therapy-responsive sample.

In another embodiment, the method comprises steps to detect cancer stem cells (CSCs) that would respond to IS-CSC or alternatively iCM being: 1) obtaining DNA from biopsied human tissue, 2) converting unmethylated cytosine residues to uracil using bisulfite, 3) digestion of DNA sample with methylation-specific restriction enzymes, 4) PCR amplifying sequences within the DMR region to determine whether the % methylation of the CpGs in the DMR or multiple DMRs is statistically-significant higher levels than normal tissue or Cf-DNA, 4) diagnosing therapy-resistant CSCs based on statistically-significantly lower methylation in the DMRs of the sample compared to a therapy-responsive sample.

In another embodiment, the method comprises steps to score the completeness of in vitro reprogramming of somatic cells to pluripotency (iPS cells) being: 1) obtaining DNA from cells treated with agents intended to reprogram somatic cells to pluripotency, 2) measuring the levels of methylated or unmethylated DNA within DMRs of the present invention, 3) determining whether the % methylation of the CpGs in the DMR or multiple DMRs is statistically-significant higher levels than normal hES cell DNA, 4) scoring the completeness of reprogramming utilizing the percentage of CpGs that are methylated within said DMRs.

In another embodiment, the method comprises steps to score the completeness of in vitro reprogramming of somatic cells to pluripotency (iPS cells) being: 1) obtaining DNA from cells treated with agents intended to reprogram somatic cells to pluripotency, 2) converting unmethylated cytosine residues to uracil using bisulfite, 3) sequencing the DMRs of the present invention to determine whether the % methylation of the CpGs in the DMR or multiple DMRs is statistically-significantly lower levels than normal pluripotent stem cells (hES cells), 4) scoring the completeness of reprogramming utilizing the percentage of CpGs that are methylated within said DMRs.

In another embodiment, the method comprises steps to score the completeness of in vitro reprogramming of somatic cells to pluripotency (iPS cells) being: 1) obtaining DNA from cells treated with agents intended to reprogram somatic cells to pluripotency, 2) converting unmethylated cytosine residues to uracil using bisulfite, 3) digestion of DNA sample with methylation-specific restriction enzymes, 4) PCR amplifying sequences within the DMR region to determine whether the % methylation of the CpGs in the DMR or multiple DMRs is statistically-significant higher levels than normal human pluripotent stem cells (hES cells), 4) scoring the completeness of reprogramming utilizing the percentage of CpGs that are methylated within said DMRs.

In another embodiment, the method comprises steps to score the extent of in vitro reprogramming of somatic cells to a pre-EFT state to reverse aging and induce tissue regeneration (iTR) being: 1) obtaining DNA from cells treated with agents intended to reverse aging and induce tissue regeneration, 2) measuring the levels of methylated or unmethylated DNA within DMRs of the present invention, 3) determining whether the % methylation of the CpGs in the DMR or multiple DMRs is statistically-significant higher levels than normal hES cell DNA, 4) scoring the completeness of iTR reprogramming utilizing the percentage of CpGs that are methylated within said DMRs.

In another embodiment, the method comprises steps to score the extent of in vivo reprogramming of somatic cells to a pre-EFT state to reverse aging and induce tissue regeneration (iTR) being: 1) obtaining DNA from cells, tissues, or body fluids treated with agents intended to reverse aging and induce tissue regeneration, 2) measuring the levels of methylated or unmethylated DNA within DMRs of the present invention, 3) determining whether the % methylation of the CpGs in the DMR or multiple DMRs is statistically-significant higher levels than normal hES cell DNA, 4) scoring the completeness of iTR reprogramming utilizing the percentage of CpGs that are methylated within said DMRs.

In another embodiment, the method comprises steps to score the extent of in vivo reprogramming of somatic cells to a pre-EFT state to reverse aging and induce tissue regeneration (iTR) being: 1) obtaining DNA from cells, tissues, or body fluids treated with agents intended to reverse aging and induce tissue regeneration, 2) converting unmethylated cytosine residues to uracil using bisulfite, 3) sequencing the DMRs of the present invention to determine whether the % methylation of the CpGs in the DMR or multiple DMRs is statistically-significantly lower levels than normal pluripotent stem cells (hES cells) and/or higher than somatic cell counterparts, and 4) scoring the completeness of iTR reprogramming utilizing the percentage of CpGs that are methylated within said DMRs.

In another embodiment, the method comprises steps to score the extent of in vivo reprogramming of somatic cells to a pre-EFT state to reverse aging and induce tissue regeneration (iTR) being: 1) obtaining DNA from cells, tissues, or body fluids treated with agents intended to reverse aging and induce tissue regeneration, 2) converting unmethylated cytosine residues to uracil using bisulfite, 3) converting unmethylated cytosine residues to uracil using bisulfite, 4) digestion of DNA sample with methylation-specific restriction enzymes, 5) PCR amplifying sequences within the DMR region to determine whether the % methylation of the CpGs in the DMR or multiple DMRs is statistically-significantly lower levels than normal human pluripotent stem cells (hES cells) and/or higher than normal somatic controls, and 4) scoring the completeness of iTR reprogramming utilizing the percentage of CpGs that are methylated within said DMRs.

In accordance with the present invention, there is provided a method for the detection or monitoring of the developmental stage of cells using a biological sample selected from cultured cells, tissue, tumors, blood, plasma, serum, saliva, urine from an individual, said method comprising:
(a) obtaining DNA from the said biological sample;
(b) determining the percent methylation of the CpG residues within the DMRs of the present invention
(c) using the percent methylation to determine whether the DNA represents an embryonic (pre-fetal) or adult epigenetic state.

The novelty of the present invention relates to novel DMRs that robustly discriminate between DNA originating from cells with an embryonic or embryo-onco phenotype as well as the novel uses of said information to diagnose cancer, determine the presence of cancer stem cells, to monitor the completeness of the in vitro reprogramming of somatic cells to pluripotency (iPS cells), and to monitor the extent of in vivo reprogramming of human cells and tissues in vivo to induce tissue regeneration (iTR). Downstream methods to detect differentially-methylated DNA, such as for applications in liquid biopsy to detect cancer-derived cfDNA (circulating tumor-derived DNA (ctDNA) are well known in the art. By way of nonlimiting example, altered methylation of the DMRs may be detected by:
(a) obtaining DNA from the said biological sample;
(b) digesting the DNA sample with one or more methylation-sensitive restriction enzymes;
(c) quantifying or detecting a DNA sequence of interest after step (b), wherein the target sequence of interest contains at least two methylation-sensitive restriction enzyme recognition sites; and
(d) comparing the level of the DNA sequence from the individual to a normal standard, to detect, prognosticate or monitor cancer.

In a preferred aspect of the present invention, the polymerase chain reaction is used in step (c). Preferably, the methylation-sensitive restriction enzyme recognizes DNA sequences which have not been methylated. The target sequence is a sequence susceptible to methylation in cancer patients so that an unmethylated target sequence in a normal patient is digested and is not amplified by the polymerase chain reaction, whereas in a cancer patient, the target sequence is methylated and is not digested by the enzyme and can subsequently be quantified or detected, for example using the polymerase chain reaction.

The methods of the present invention can be used to predict the susceptibility to cancer of the individual, to assess the stage of cancer in the individual, to predict the likelihood of overall survival for the individual, to predict the likelihood of recurrence for the individual or to assess the effectiveness of treatment in the individual.

In accordance with another aspect of the present invention, there is provided a method for the detection or monitoring of cancer using a biological sample selected from tissue, tumor, blood, plasma, serum, saliva, urine from an individual, said method comprising:
(a) obtaining DNA from the said biological sample;
(b) digesting the DNA sample with one or more methylation-sensitive restriction enzymes;
(c) quantifying or detecting a DNA sequence of interest after step (b) wherein the DNA sequence is a sequence comprising part or all of a DMR in Table I; and
(d) comparing the level of the DNA sequence from the individual to a normal standard, to detect, prognosticate or monitor cancer.

Methods for the PCR-based amplification and detection of DMRs such as with Luminometric Methylation Assay (LUMA), bisulfite conversion, pyrosequencing, mass spectrometry, qPCR arrays, affinity and restriction enzyme-based arrays, bisulfite conversion-based arrays, and next generation sequencing are well-known in the art (Kurdyukov, S. and Bullock, M. DNA Methylation Analysis: Choosing the Right Method. 2016 Biology 5(1):3 and Sant, K. E. et al, DNA Methylation Screening and Analysis. 2012. Methods Mol Bio 889: 385-406) incorporated herein by reference with the general rules being generally applicable:

In accordance with a further aspect of the invention, there is provided probes, primers and kits for use in the method of the invention. In particular, there is provided:
a set of primers for the detection or monitoring of cancer in a biological sample selected from tissue, tumors, blood, plasma, serum, saliva, urine from an individual, which comprises a primers specific for the DMRs of Table I wherein the primer sets are shown in Table II;
a kit for the detection or monitoring of cancer in a biological sample selected from tissue, tumors, blood, plasma, serum, saliva, urine from an individual, which comprises the probe of the invention and the set of primers of the invention; and
a kit for use as a control during the detection or monitoring of cancer in a biological sample selected from blood, plasma, serum, saliva, urine from an individual, which comprises the primer sets of the invention and the set of control primers of the invention.

### DESCRIPTION OF THE FIGURES

FIG. 1 IGV of methylated CpG residues displayed as percent modified. Shown are four hES cell-derived clonal embryonic progenitor cell lines corresponding to osteogenic mesenchyme, vascular endothelium, skeletal myoblasts, and white preadipocytes respectively (4D20.8, 30-MV2-6, SK5, and E3) followed by their respective four adult-derived counterparts (bone marrow mesenchymal stem cells (MSCs), aortic endothelial cells (HAEC), skeletal myoblasts, and white preadipocytes respectively. The position of DMR_327 is shown as the bar in the row labeled Top DMRs.

FIG. 2 shows IGV of ATAC-seq and CpG methylation results of two hES cell-derived clonal embryonic progenitor cell lines corresponding to osteogenic mesenchyme and vascular endothelium (4D20.8 and 30-MV2-6 respectively) followed by their respective two adult-derived counterparts (bone marrow mesenchymal stem cells (MSCs) and aortic endothelial cells (HAEC). Also shown are comparable ATAC and BIS results from the two hES cell lines MA03 and H9 and iPSCs produced from what were originally the hES cell-derived clonal cell line EN13 (Vaziri et al 2010, Spontaneous reversal of the developmental aging of normal human cells following transcriptional reprogramming Regen Med 5(3):345-363), as well as human adult dermal fibroblast-derived iPSCs. The row titled Top DMRs shows the location of the DMR.

FIG. 3 shows IGV of CpG methylation results obtained by BIS of a hES cell-derived clonal embryonic progenitor cell line corresponding to osteogenic mesenchyme and a normal adult counterpart being bone marrow mesenchymal stem cells (4D20.8 and MSCs respectively) followed by corresponding adult-derived cancer cell lines derived from osteogenic mesenchyme (the osteosarcoma cell lines U-2, SJSA-1, KHOS-240S, and KHOS/NP). Also shown are CpG methylation results obtained by BIS of a hES cell-derived clonal embryonic progenitor cell line corresponding to skeletal myoblasts and an adult derived normal counterpart being adult skeletal myoblasts (SK5 and Adult Skel Muscle Myoblasts respectively) followed by corresponding adult-derived cancer cell lines derived from muscle mesenchyme (the rhabdomyosarcoma (RMS) cell lines CCL-136, A-204, SJCRH30, and TE 617.T). Also shown are CpG methylation results obtained by BIS of a hES cell-derived clonal embryonic progenitor cell line corresponding to white adipocyte progenitors (E3) and an adult derived normal counterpart being adult preadipocytes and the lipogenic cancer cell lines (94T778 and 93T449). The row titled Top DMRs shows the location of the DMR.

FIG. 4 shows RNA-seq values in FPKM of the transcript LINC00865 in four hES cell lines and an EP-derived iPS cell line (ES & iPSC); 42 diverse hESC-derived EP cell lines (Diverse EPs); 100 diverse somatic cell types including neuronal, glial, hepatocytes, diverse stromal cell types as well as others (Diverse Normal Somatic Cells); 24 diverse cultured epithelial cell types (Epithelial); 39 diverse sarcoma cell types (Sarcomas); 35 diverse carcinoma and adenocarcinoma cell types (Carcinomas); and four blood cancer cell types (Blood CA).

FIG. 5 shows RNA-seq values in FPKM of the transcript LINC00865 in four hES cell lines and an EP-derived iPS cell line (ES & iPSC); three dermal fibroblast cultures from the upper arm of late embryonic (8 wk) human embryos (Emb); 12 dermal fibroblast cultures from the upper arm of human fetuses aged 9-16 wk (Fetal); 13 dermal fibroblast cultures from the upper arm of human neonates aged 3-13 years (Neonatal); and 29 dermal fibroblast cultures from the upper arm of human adults aged 19-83 years (Old Age); as well as human adult fibroblasts from a 59 year old donor (passage 5) from the upper arm cultured in conditions to induce quiescence as described herein along with iPS cells generated from said fibroblasts (passage 6) labelled (Old & Reprogrammed).

FIG. 6 shows RNA-seq values in FPKM in the hES cell lines H9, MA03, ESI 017, ESI 053, and the EP-derived iPS cell line EH3 as well as an adult-derived (59 year-old) dermal fibroblast line followed by an iPS cell line derived from said adult fibroblasts of the transcripts: A) DNMT3B; B) POU5F1 (OCT4); C) LIN28B; and the fetal/adult-onset gene PCDHGA12.

FIG. 7: IGV image of the region surrounding DMR_327 and the gene LINC00865. From top to bottom, rows show BIS-generated CpG methylation for the osteogenic mesenchymal EP cell line 4D20.8 followed by its adult counterpart bone marrow-derived MSCs; CTCF binding sites (none in this example); DMR Q-values followed by the significance ranking of the top 1000 DMRs; ChIP-seq reads for the indicated histone modifications in the embryonic versus adult cells.

FIG. 8: The fraction of the CpGs methylated in DMR_327 in colon cancer vs normal colon, prostate cancer vs benign prostate, glioblastoma with an IDH mutation vs normal brain, glioblastoma with an MSC phenotype vs normal brain, glioblastoma with a PDGFRA amplification vs normal brain, glioblastoma with an EGFR amplification vs normal brain, liposarcoma cells vs normal subcutaneous preadipocytes, osteosarcoma cells vs bone marrow MSCs, and rhabdomyosarcoma vs normal skeletal muscle myoblasts. Each in replicate.

FIG. 9: Timeline of the growth of xenograft tumors from the fibrosarcoma cell line HT1080 and HT1080 exogenously expression adult levels of COX7A1.

FIG. 10: Expression of the adult cell markers COX7A1 and CAT in pancreatic tumor vs surviving pancreatic cancer stem cells following KRAS ablation.

FIG. 11: Apoptotic response as measured by the TUNEL assay of the DC fibrosarcoma cell line HT1080 following exogenous expression of the iCM gene COX7A1. TABLE I shows the DMRs hypermethylated in pre-EFT cells and DC cells of the present invention, together with their unique status wherein said status marked with an asterisk (“*”) are novel over Su et al, 2018); those without the asterisk are covered in at least 2 nucleotides with DMRs disclosed in Su et al, 2018, chromosome number (Chr), start and end position on the designated chromosome in human genome Hg38; the size of the DMR region in bp, the statistical significance (Q-value) of the differential methylation as determined in four hES cell-derived clonal embryonic progenitors lines (the osteogenic mesenchyme line 4D20.8, the endothelial line 30-MV2-6, the preadipocyte line E3, and the skeletal myoblast line SK-5, compared to their adult counterparts bone marrow MSCs, aortic endothelial cells, subcutaneous white preadipocytes, and skeletal muscle myoblasts); the % methylation difference between the average pre-EFT lines and adult, and the number of CpGs in the DMR. The DMRs with an asterisk in Table I (the ones not in Su et al 2018) are disclosed as part of the invention for both determining the EFT status and making choices thereby for therapy as well as for detecting cancer generally such as with liquid biopsy. DMRs without the asterisk are disclosed by Su et al 2018 and are disclosed as part of the invention for determining EFT status and subsequent therapy decisions.

### DETAILED DESCRIPTION OF THE INVENTION

### Abbreviations

AC Cells—Adult Cancer cells refers to malignant cancer cells that display post-EFT epigenetic markers such as the relatively unmethylated DMRs of the present invention.
AMH—Anti-Mullerian Hormone
ATAC—Assay for Transposase Accessible Chromatin
ATAC-seq—Assay for Transposase Accessible Chromatin followed by high throughput DNA sequencing
ASC—Adult stem cells
BIS—Bisulfite sequencing refers to the sequencing of DNA subsequent to the bisulfite modification of unmethylated cytosines to uracils as a means of identifying methylated CpGs.
BP—Base pairs of DNA
Chr—Chromosome
CSC—Cancer Stem Cell
cGMP—Current Good Manufacturing Processes
CM—Cancer Maturation
CNS—Central Nervous System
CTCF—CCCTC-binding factor
cfDNA—Cell-free DNA
ctDNA—Circulating tumor-derived DNA
DC Cells—Dematured Cancer Cells refer to normal cells that have acquired in the course of oncogenesis a pre-EFT pattern of gene expression and a pre-EFT pattern of heavily methylated DMRs of the present invention
DMEM—Dulbecco's modified Eagle's medium
DMR—Differentially-Methylated Region refers to CpGs that are significantly differentially methylated in pre-EFT cells compared to Post-EFT cells.
DPBS—Dulbecco's Phosphate Buffered Saline
ED Cells—Embryo-derived cells; hED cells are human ED cells
EDTA—Ethylenediamine tetraacetic acid
EFT—Embryonic-Fetal Transition being the developmental transition that occurs in humans at the completion of 8 weeks of gestation when fetal development commences.
EG Cells—Embryonic germ cells; hEG cells are human EG cells
EP—Embryonic progenitors
ES Cells—Embryonic stem cells; hES cells are human ES cells
ESC—Embryonic Stem Cells
FACS—Fluorescence activated cell sorting
FBS—Fetal bovine serum
FPKM—Fragments Per Kilobase of transcript per Million mapped reads from RNA sequencing.
hED Cells—Human embryo-derived cells
hEG Cells—Human embryonic germ cells are stem cells derived from the primordial germ cells of fetal tissue.
HESC—Human Embryonic Stem Cells
hiPS Cells—Human induced pluripotent stem cells are cells with properties similar to hES cells obtained from somatic cells after exposure to hES-specific transcription factors such as SOX2, KLF4, OCT4, MYC, or NANOG, LIN28, OCT4, and SOX2.
iCM—Induced Cancer Maturation.
IGV—Integrative Genomics Viewer
iPS Cells—Induced pluripotent stem cells are cells with properties similar to hES cells obtained from somatic cells after exposure to ES-specific transcription factors such as SOX2, KLF4, OCT4, MYC, or NANOG, LIN28, OCT4, and SOX2, SOX2, KLF4, OCT4, MYC, and (LIN28A or LIN28B), or other combinations of OCT4, SOX2, KLF4, NANOG, ESRRB, NR5A2, CEBPA, MYC, LIN28A and LIN28B.
iS—induced Senolysis refers to the use of iTR to induce the intrinsic apoptosis of aged or senescent cells.
iS-CSC—induced Senolysis of Cancer Stem Cells refers to the treatment of cells in malignant tumors that are refractory to ablation by chemotherapeutic agents or radiation therapy wherein said iS-CSC treatment causes said refractory cells to revert to a pre-fetal pattern of gene expression and become sensitive to chemotherapeutic agents or radiation therapy.
iTM—Induced Tissue Maturation
iTR—Induced Tissue Regeneration
MEM—Minimal essential medium
MSCs—Mesenchymal stem cells
MSP—Methylation specific PCR
NT—Neonatal transition which is the developmental transition of the conceptus at the time of parturition.
PBS—Phosphate buffered saline
RFU—Relative Fluorescence Units
RMS—Rhabdomyosarcoma
RNA-seq—RNA sequencing
SFM—Serum-Free Medium

The present invention provides a method to assess, diagnose, prognosticate or monitor the presence or progression of tumors in an individual including but not limited to predicting the sensitivity of cancer cells to chemotherapeutic agents or iCM protocols. Surprisingly, such diagnosis is pan-cancer in nature and relates to a broad array of cancer types including carcinomas, adenocarcinomas, and sarcomas, including but not limited to hepatocellular carcinomas, epidermoid carcinomas, renal cell adenocarcinomas, colorectal carcinomas and adenocarcinomas, bronchio-alveolar carcinomas such as non-small cell lung cancer, mammary gland carcinomas, mammary ductal carcinomas; vaginal and cervical carcinomas, gastric carcinomas, prostate carcinomas and adenocarcinomas, uterine adenocarcinomas); embryonal neuroectodermal tumors and teratocarcinomas; brain cell cancers such as glioblastomas and neuroblastomas; blood cell cancers such as histiocytic and lymphoblastic lymphomas and B-cell lymphoblastic leukemia; or sarcomas (including but not limited to embryonic and alveolar rhabdomyosarcomas, osteosarcomas, chondrosarcomas, liposarcomas, giant cell sarcomas of bone, uterine sarcomas, leiomyosarcomas, Wilms tumor, Ewing's sarcoma, pagetoid sarcoma, epithelioid sarcoma, synovial sarcomas, fibrosarcomas, and spindle cell sarcomas).

In cases where these as other carcinomas, adenocarcinomas, sarcomas, and brain or blood cell cancers have been determined by means of the methods of the present invention have reverted to an embryonic phenotype (also known as an embryo-onco phenotype) (also known as Dematured Cancer (DC) cells), then treating a patient's cancer with agents appropriate to that phenotype, i.e. agents that are effective in inhibiting the replication or inducing apoptosis of the cancer cells in that particular phenotype such as common chemotherapeutic agents including but not limited to the alkylating agents including but not limited to altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, or trabectedin can be employed. Alternatively, using the compositions and methods of the instant invention, CNS tumors such as glioblastomas and astrocytomas that display a pre-fetal embryo-onco phenotype (DC Cells) may be selected for treatment with alkylating agents that cross the blood-brain barrier such as carmustine, lomustine, and streptozocin. In addition, using the compositions and methods of the instant invention, tumors that display a pre-fetal embryo-onco phenotype (DC cells) are determined to proliferate at a relatively fast rate and to metastasize more aggressively than those that display a fetal or adult phenotype (Adult Cancer (AC) cells), therefore said DC cells, and are determined to be more sensitive to antimetabolites including but not limited to azacytidine, 5-fluorouracil (5-FU), 6-mercaptopurine (6-MP), capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, and trifluridine. Alternatively, using the compositions and methods of the instant invention, tumors that display a pre-fetal embryo-onco phenotype (DC cells) and are therefore determined to proliferate at a relatively fast rate and to metastasize more aggressively than cells are determined to be more sensitive to anti-tumor antibiotics including but not limited to the anthracyclines daunorubicin, doxorubicin, epirubicin, idarubicin, and valrubicin or bleomycin, dactinomycin, mitomycin-C, and mitroxantrone, or the topoisomerase inhibitors irinotecan, topotecan, camptothecin, etoposide, teniposide, the mitotic inhibitors cabazitaxel, docetaxel, nab-pacitaxel, and paclitaxel, or the vinca alkyloids vinblastine, vincristine, and vinorelbine.

In addition, or in contrast, in cases where malignant cells have reverted to a post-EFT phenotype (AC cells) (surprisingly also known as what are commonly designated cancer stem cells), thereby becoming relatively resistant to apoptosis, the resistant “cancer stem cells” can be induced back to a pre-fetal phenotype to increase their susceptibility to treatments that induce apoptosis. These include the reprogramming of said AC cells using iTR reprogramming methods disclosed herein (also known as the induction of senolysis of cancer stem cells (iS-CSC), inhibiting the PI3K/AKT/MTOR (phosphoinositide 3-kinase/AKT/namimalian target of rapamycin) pathway such as with rapanycin or other inhibitors of mTOR, dietary restriction, or the use of dietary restriction mimetics. These and related uses of pathways related to the EFT in the diagnosis and treatment of cancer are the subject of the present invention.

It is known in the art that numerous tumor suppressor genes are relatively highly methylated in many tumor cells. Therefore, the use of such highly methylated circulating tumor DNA (ctDNA) is known in the art to be useful as diagnostic and prognostic markers for managing cancer in animals including humans. However, there remains a need to identify additional such markers of cancer, in particular, those that are markers of all cancer types (pan-cancer markers), and those that can support clinical decision making in the choice of optimum therapeutic strategies. The present invention provides a large number of novel DMRs identified through a comparative analysis of regions differentially methylated in four hES cell-derived clonal embryonic progenitors to osteochondral mesenchyme, vascular endothelium, skeletal myoblasts, and white preadipocytes compared to their adult counterparts. The positions of these DMRs in the Hg38 version of the human genome are shown in Table I. The DMRs are listed in rank order of the statistical significance of the differential methylation, such significance being <1×10E-25 for the entire list.

TABLE I

| | | | | | | | Mean | |
|---|---|---|---|---|---|---|---|---|
| | | | | | Region | | Methylation | |
| Unique | DMR | | | | Size | | Difference | Number |
| Status | Name | chr | Start | Stop | (bp) | Q-value | (%) | CpGs |
| | DMR_001 | chr19 | 2250699 | 2252101 | 1402 | 2.80E−251 | 87.43 | 253 |
| | DMR_002 | chr22 | 46074866 | 46080081 | 5215 | 5.50E−203 | 77.28 | 279 |
| | DMR_003 | chr5 | 42951061 | 42952709 | 1648 | 4.60E−187 | 82.15 | 211 |
| | DMR_004 | chr19 | 58355899 | 58357477 | 1578 | 7.70E−180 | 78.17 | 205 |
| | DMR_005 | chr19 | 45152055 | 45153440 | 1385 | 9.60E−153 | 85.33 | 172 |
| * | DMR_006 | chr5 | 141418258 | 141420380 | 2122 | 2.00E−152 | 63.99 | 189 |
| | DMR_007 | chr22 | 46084688 | 46087931 | 3243 | 8.50E−149 | 71.41 | 222 |
| | DMR_008 | chr17 | 17205477 | 17206497 | 1020 | 3.70E−140 | 86.73 | 163 |
| | DMR_009 | chr5 | 141430419 | 141431777 | 1358 | 7.30E−133 | 81.36 | 146 |
| * | DMR_010 | chr5 | 141431781 | 141433256 | 1475 | 2.70E−129 | 60.74 | 183 |
| | DMR_011 | chr1 | 200873014 | 200874207 | 1193 | 5.60E−129 | 76.44 | 164 |
| * | DMR_012 | chr5 | 141101671 | 141103356 | 1685 | 9.60E−125 | 61.06 | 189 |
| | DMR_013 | chr14 | 64541526 | 64542606 | 1080 | 1.90E−124 | 76.79 | 153 |
| | DMR_014 | chr6 | 26043041 | 26045991 | 2950 | 1.80E−121 | 61.54 | 228 |
| | DMR_015 | chr22 | 46053155 | 46056587 | 3432 | 4.30E−121 | 77.27 | 172 |
| * | DMR_016 | chr2 | 131039692 | 131040648 | 956 | 2.50E−120 | 61.33 | 152 |
| | DMR_017 | chr1 | 2789309 | 2790366 | 1057 | 2.60E−117 | 72.46 | 165 |
| | DMR_018 | chr1 | 147078724 | 147080452 | 1728 | 4.30E−115 | 51.79 | 234 |
| | DMR_019 | chr5 | 141421454 | 141423647 | 2193 | 2.50E−114 | 59.43 | 190 |
| * | DMR_020 | chr5 | 141409467 | 141410141 | 674 | 9.10E−114 | 77.04 | 123 |
| * | DMR_021 | chr4 | 1471639 | 1472662 | 1023 | 1.60E−112 | 83.75 | 128 |
| | DMR_022 | chr12 | 1796381 | 1797560 | 1179 | 3.10E−112 | 61.39 | 192 |
| | DMR_023 | chr7 | 99918960 | 99919785 | 825 | 1.80E−110 | 81.84 | 134 |
| | DMR_024 | chr8 | 56112946 | 56114042 | 1096 | 3.00E−109 | 73.43 | 147 |
| | DMR_025 | chr8 | 143275827 | 143276642 | 815 | 3.10E−108 | 76.53 | 143 |
| | DMR_026 | chr10 | 103584327 | 103585102 | 775 | 1.80E−107 | 64.14 | 199 |
| | DMR_027 | chr1 | 7953758 | 7954617 | 859 | 4.20E−107 | 79.13 | 130 |
| | DMR_028 | chr7 | 157612781 | 157613782 | 1001 | 9.10E−107 | 75.26 | 140 |
| | DMR_029 | chr3 | 42685716 | 42686423 | 707 | 9.60E−107 | 74.13 | 138 |
| | DMR_030 | chr2 | 240672528 | 240673179 | 651 | 2.30E−105 | 78.28 | 132 |
| * | DMR_031 | chr2 | 241803131 | 241804698 | 1567 | 2.70E−105 | 57.25 | 268 |
| | DMR_032 | chr14 | 69572579 | 69573684 | 1105 | 4.50E−105 | 66.99 | 150 |
| * | DMR_033 | chr5 | 141136703 | 141137837 | 1134 | 8.70E−105 | 53.52 | 174 |
| | DMR_034 | chr4 | 6663861 | 6664923 | 1062 | 1.40E−104 | 80.52 | 125 |
| * | DMR_035 | chrX | 1465424 | 1466337 | 913 | 1.40E−101 | 74.93 | 135 |
| | DMR_036 | chr7 | 64569488 | 64570399 | 911 | 7.30E−101 | 79.72 | 123 |
| | DMR_037 | chr15 | 52112085 | 52112836 | 751 | 2.90E−100 | 77.07 | 129 |
| * | DMR_038 | chr19 | 36151147 | 36153031 | 1884 | 1.10E−99 | 78.83 | 123 |
| | DMR_039 | chr2 | 232351797 | 232352767 | 970 | 1.60E−98 | 80.38 | 114 |
| | DMR_040 | chr5 | 73381388 | 73382712 | 1324 | 1.80E−98 | 58.08 | 192 |
| | DMR_041 | chr19 | 23070349 | 23071580 | 1231 | 2.70E−98 | 74.39 | 137 |
| | DMR_042 | chr7 | 56115839 | 56116630 | 791 | 1.00E−94 | 73.12 | 134 |
| * | DMR_043 | chr8 | 144109493 | 144110147 | 654 | 6.20E−93 | 78.76 | 94 |
| * | DMR_044 | chr5 | 141389221 | 141390111 | 890 | 1.30E−92 | 57.79 | 138 |
| | DMR_045 | chr8 | 143707138 | 143708079 | 941 | 1.80E−92 | 63.25 | 127 |
| | DMR_046 | chr18 | 14450469 | 14450984 | 515 | 4.40E−92 | 76.39 | 125 |
| | DMR_047 | chr9 | 97076627 | 97077677 | 1050 | 1.10E−91 | 70.92 | 129 |
| * | DMR_048 | chr1 | 245687575 | 245688591 | 1016 | 1.50E−91 | 55.58 | 125 |
| | DMR_049 | chr10 | 70255234 | 70255922 | 688 | 2.50E−91 | 78.06 | 112 |
| | DMR_050 | chr9 | 123373057 | 123373951 | 894 | 2.70E−91 | 75.94 | 121 |
| * | DMR_051 | chr7 | 143510972 | 143511764 | 792 | 5.40E−90 | 60.51 | 115 |
| | DMR_052 | chr8 | 39106981 | 39107994 | 1013 | 8.00E−90 | 76.57 | 116 |
| | DMR_053 | chr2 | 201115668 | 201117061 | 1393 | 1.30E−89 | 38.57 | 176 |
| * | DMR_054 | chr11 | 95037499 | 95038028 | 529 | 1.30E−87 | 70.78 | 114 |
| * | DMR_055 | chr5 | 172669735 | 172670339 | 604 | 3.40E−87 | 59.52 | 99 |
| * | DMR_056 | chr5 | 141096095 | 141097146 | 1051 | 3.10E−86 | 60.82 | 126 |
| | DMR_057 | chr19 | 940381 | 941556 | 1175 | 5.90E−86 | 68.83 | 117 |
| * | DMR_058 | chr2 | 39243601 | 39244144 | 543 | 1.40E−85 | 53.76 | 107 |
| | DMR_059 | chr19 | 58009360 | 58010023 | 663 | 5.00E−85 | 84.74 | 99 |
| | DMR_060 | chr5 | 157670565 | 157672009 | 1444 | 1.00E−84 | 57.76 | 184 |
| * | DMR_061 | chr19 | 58349937 | 58351032 | 1095 | 4.20E−84 | 79.91 | 98 |
| * | DMR_062 | chr19 | 58203919 | 58204665 | 746 | 2.00E−83 | 80.45 | 92 |
| | DMR_063 | chr17 | 76075849 | 76077796 | 1947 | 2.70E−83 | 48.89 | 264 |
| * | DMR_064 | chr3 | 126541724 | 126542341 | 617 | 3.70E−83 | 71.56 | 97 |
| * | DMR_066 | chr5 | 140823132 | 140823943 | 811 | 3.30E−82 | 47.38 | 137 |
| | DMR_067 | chr6 | 10719884 | 10720978 | 1094 | 5.30E−82 | 68.23 | 103 |
| * | DMR_068 | chr18 | 80159732 | 80160128 | 396 | 7.20E−82 | 73.18 | 91 |
| | DMR_069 | chr4 | 143911990 | 143912359 | 369 | 2.40E−81 | 78.92 | 99 |
| | DMR_070 | chr2 | 169768217 | 169768970 | 753 | 4.20E−81 | 74.31 | 102 |
| | DMR_071 | chr14 | 67411817 | 67412601 | 784 | 4.90E−81 | 71.93 | 123 |
| * | DMR_072 | chr5 | 141414488 | 141415677 | 1189 | 5.00E−81 | 62.69 | 112 |
| * | DMR_073 | chr7 | 102300503 | 102300918 | 415 | 1.20E−80 | 70.87 | 91 |
| | DMR_074 | chr19 | 58561830 | 58563850 | 2020 | 3.00E−80 | 50.38 | 255 |
| | DMR_075 | chr11 | 89863850 | 89864607 | 757 | 4.20E−80 | 61.36 | 133 |
| | DMR_076 | chr13 | 52194956 | 52195816 | 860 | 1.20E−79 | 82.83 | 96 |
| | DMR_077 | chr5 | 177400159 | 177400636 | 477 | 1.60E−79 | 82.46 | 99 |
| | DMR_078 | chr17 | 1270473 | 1271492 | 1019 | 2.40E−79 | 73.83 | 116 |
| | DMR_079 | chr3 | 8767331 | 8767850 | 519 | 2.50E−79 | 77.71 | 93 |
| | DMR_080 | chr14 | 33799794 | 33800749 | 955 | 2.70E−79 | 61.26 | 128 |
| * | DMR_081 | chr3 | 44580918 | 44581374 | 456 | 2.00E−78 | 66.47 | 94 |
| * | DMR_082 | chr5 | 140870342 | 140870983 | 641 | 6.40E−78 | 57.84 | 122 |
| | DMR_083 | chr10 | 133528096 | 133528853 | 757 | 7.10E−78 | 63.25 | 121 |
| * | DMR_084 | chr1 | 11501401 | 11501896 | 495 | 8.80E−78 | 61.32 | 115 |
| | DMR_085 | chr16 | 54938153 | 54940011 | 1858 | 9.00E−78 | −37.47 | 188 |
| | DMR_086 | chr2 | 9474066 | 9475564 | 1498 | 1.00E−77 | 48.42 | 192 |
| * | DMR_087 | chr22 | 50577791 | 50578738 | 947 | 1.00E−77 | 72.59 | 112 |
| | DMR_088 | chr9 | 136994737 | 136995891 | 1154 | 1.30E−77 | 60.87 | 125 |
| * | DMR_089 | chr5 | 141151648 | 141152632 | 984 | 2.90E−77 | 60.81 | 121 |
| | DMR_090 | chr7 | 158957546 | 158958855 | 1309 | 7.90E−77 | 59.58 | 140 |
| * | DMR_091 | chr2 | 94272682 | 94273712 | 1030 | 1.20E−76 | 83.17 | 92 |
| | DMR_092 | chr4 | 84482243 | 84484227 | 1984 | 4.50E−76 | 48.05 | 212 |
| | DMR_093 | chr5 | 177984212 | 177984925 | 713 | 4.50E−76 | 69.93 | 117 |
| * | DMR_094 | chr2 | 94208342 | 94208946 | 604 | 8.10E−76 | 79.23 | 110 |
| | DMR_095 | chr13 | 109140460 | 109141351 | 891 | 9.90E−76 | 78.58 | 101 |
| | DMR_096 | chr17 | 57045464 | 57046363 | 899 | 1.30E−75 | 69.01 | 123 |
| | DMR_097 | chr7 | 56174725 | 56175831 | 1106 | 1.30E−75 | 69.68 | 110 |
| | DMR_098 | chr17 | 82374854 | 82375645 | 791 | 1.70E−75 | 71.63 | 108 |
| | DMR_099 | chr1 | 227560723 | 227561531 | 808 | 1.90E−75 | 69.48 | 104 |
| | DMR_100 | chr10 | 86968316 | 86969034 | 718 | 3.80E−74 | 79.49 | 94 |
| * | DMR_1000 | chr2 | 47071890 | 47072651 | 761 | 2.80E−26 | 59.06 | 54 |
| | DMR_101 | chr19 | 12194498 | 12195811 | 1313 | 4.60E−74 | 52.53 | 165 |
| * | DMR_102 | chr5 | 141399596 | 141400261 | 665 | 6.50E−74 | 70.83 | 91 |
| * | DMR_103 | chr7 | 47052646 | 47053139 | 493 | 9.40E−74 | 71.29 | 79 |
| | DMR_104 | chr1 | 6455459 | 6456143 | 684 | 1.80E−73 | 73.36 | 102 |
| | DMR_105 | chr14 | 24310511 | 24311647 | 1136 | 2.60E−73 | 59.21 | 162 |
| | DMR_106 | chr14 | 96592402 | 96593301 | 899 | 9.40E−73 | 72.89 | 102 |
| * | DMR_107 | chr5 | 141394417 | 141395515 | 1098 | 1.30E−72 | 57.95 | 110 |
| | DMR_108 | chr1 | 6454650 | 6455084 | 434 | 2.10E−72 | 88.39 | 88 |
| | DMR_109 | chr2 | 91441994 | 91443601 | 1607 | 3.80E−72 | 48.48 | 191 |
| * | DMR_110 | chr16 | 817208 | 817833 | 625 | 6.00E−72 | 63.53 | 117 |
| * | DMR_111 | chr19 | 2275695 | 2276478 | 783 | 1.50E−71 | 58.43 | 113 |
| | DMR_112 | chr7 | 73769205 | 73769846 | 641 | 2.80E−71 | 67.65 | 105 |
| | DMR_113 | chr13 | 20177206 | 20177572 | 366 | 9.70E−71 | 80.47 | 85 |
| | DMR_114 | chr19 | 36421102 | 36421978 | 876 | 1.80E−70 | 60.45 | 147 |
| | DMR_115 | chr7 | 153887852 | 153888508 | 656 | 1.80E−70 | 75.27 | 106 |
| | DMR_116 | chr11 | 504189 | 504937 | 748 | 2.90E−70 | 89.27 | 83 |
| | DMR_117 | chr15 | 90929733 | 90930742 | 1009 | 4.40E−70 | 81.74 | 89 |
| | DMR_118 | chr5 | 180590133 | 180591631 | 1498 | 5.20E−70 | 48.17 | 224 |
| | DMR_119 | chr15 | 100848993 | 100850180 | 1187 | 5.70E−70 | 74.89 | 103 |
| | DMR_121 | chr5 | 176060830 | 176061389 | 559 | 6.50E−70 | 75.37 | 91 |
| * | DMR_122 | chr20 | 14226364 | 14226901 | 537 | 1.20E−69 | 80.04 | 82 |
| | DMR_123 | chr1 | 71046416 | 71047179 | 763 | 1.90E−69 | 70.69 | 103 |
| | DMR_124 | chr2 | 130427660 | 130428992 | 1332 | 2.70E−69 | 49.05 | 171 |
| | DMR_126 | chr16 | 32085024 | 32086011 | 987 | 1.30E−68 | 72.39 | 98 |
| * | DMR_127 | chr7 | 64181634 | 64182257 | 623 | 1.40E−68 | 63.66 | 90 |
| * | DMR_128 | chr5 | 141361737 | 141362282 | 545 | 1.50E−68 | 70.71 | 100 |
| * | DMR_129 | chr16 | 28367833 | 28368613 | 780 | 1.60E−67 | 68.14 | 99 |
| | DMR_130 | chr6 | 17015912 | 17016390 | 478 | 2.40E−67 | 78.38 | 78 |
| | DMR_131 | chr20 | 46010585 | 46011650 | 1065 | 3.30E−67 | 72.7 | 93 |
| | DMR_132 | chr12 | 7872673 | 7873370 | 697 | 1.10E−66 | 69.64 | 99 |
| | DMR_133 | chr5 | 140632026 | 140633185 | 1159 | 1.30E−66 | 50.25 | 151 |
| | DMR_136 | chr14 | 102922694 | 102923988 | 1294 | 4.10E−66 | 55.12 | 149 |
| | DMR_137 | chr19 | 47456942 | 47457544 | 602 | 4.20E−66 | 58.87 | 129 |
| | DMR_138 | chr10 | 42897760 | 42898834 | 1074 | 1.20E−65 | 69.98 | 104 |
| | DMR_140 | chr10 | 80535745 | 80536460 | 715 | 1.90E−65 | 66.01 | 91 |
| | DMR_141 | chr20 | 44115838 | 44116417 | 579 | 1.90E−65 | 79.6 | 77 |
| * | DMR_142 | chr19 | 51098580 | 51099305 | 725 | 2.20E−65 | 45.37 | 107 |
| * | DMR_143 | chr1 | 121117977 | 121118626 | 649 | 2.30E−65 | 82.16 | 82 |
| | DMR_145 | chr11 | 47589966 | 47590201 | 235 | 2.60E−65 | 71.65 | 73 |
| | DMR_146 | chr7 | 156607615 | 156608164 | 549 | 2.80E−65 | 84.91 | 79 |
| * | DMR_147 | chr10 | 61867420 | 61868069 | 649 | 3.40E−65 | 66.07 | 96 |
| | DMR_148 | chr18 | 72541465 | 72543838 | 2373 | 3.40E−65 | 41.69 | 247 |
| | DMR_149 | chr11 | 614555 | 615450 | 895 | 3.70E−65 | 56.88 | 113 |
| | DMR_150 | chr3 | 50193312 | 50194472 | 1160 | 3.70E−65 | 60.4 | 127 |
| | DMR_151 | chr10 | 29409325 | 29410341 | 1016 | 4.50E−65 | 74.25 | 92 |
| | DMR_152 | chr2 | 47521168 | 47521700 | 532 | 5.30E−65 | 88.15 | 75 |
| | DMR_153 | chr18 | 12911027 | 12911580 | 553 | 1.30E−64 | 69.37 | 94 |
| * | DMR_154 | chr3 | 75395857 | 75396477 | 620 | 2.60E−64 | 65.17 | 97 |
| * | DMR_155 | chr20 | 34475982 | 34476591 | 609 | 4.80E−64 | 84.61 | 71 |
| | DMR_156 | chr5 | 33937621 | 33938115 | 494 | 6.00E−64 | 68.49 | 86 |
| | DMR_157 | chr6 | 1601393 | 1602007 | 614 | 9.20E−64 | 66.98 | 88 |
| | DMR_158 | chr9 | 25677165 | 25678888 | 1723 | 9.60E−64 | 45.26 | 191 |
| | DMR_159 | chr14 | 44911991 | 44912514 | 523 | 1.80E−63 | 87.39 | 65 |
| | DMR_160 | chr5 | 115962594 | 115963149 | 555 | 1.80E−63 | 77.14 | 88 |
| | DMR_161 | chr12 | 124369224 | 124370295 | 1071 | 2.90E−63 | 71.57 | 96 |
| * | DMR_162 | chr5 | 141392455 | 141393356 | 901 | 3.00E−63 | 65.65 | 87 |
| | DMR_163 | chr13 | 112330893 | 112331418 | 525 | 3.80E−63 | 73.14 | 89 |
| | DMR_164 | chr8 | 95072934 | 95073582 | 648 | 4.20E−63 | 79.48 | 78 |
| * | DMR_165 | chr6 | 169961853 | 169962565 | 712 | 6.30E−63 | 60.05 | 118 |
| * | DMR_167 | chr6 | 110399465 | 110400284 | 819 | 1.10E−62 | 71.51 | 85 |
| | DMR_168 | chr6 | 42727076 | 42727473 | 397 | 2.60E−62 | 78.21 | 78 |
| | DMR_169 | chr8 | 66542190 | 66542798 | 608 | 3.20E−62 | 76.65 | 76 |
| | DMR_170 | chr1 | 175599114 | 175599672 | 558 | 3.50E−62 | 71.93 | 84 |
| * | DMR_171 | chr7 | 76150123 | 76150808 | 685 | 3.70E−62 | 67.5 | 85 |
| | DMR_172 | chr9 | 97307470 | 97308153 | 683 | 3.80E−62 | 80.71 | 74 |
| | DMR_173 | chr8 | 43246597 | 43247293 | 696 | 5.40E−62 | 59.33 | 109 |
| * | DMR_174 | chr2 | 97724035 | 97724392 | 357 | 5.70E−62 | 70.86 | 79 |
| * | DMR_175 | chr1 | 45622529 | 45623522 | 993 | 6.70E−62 | 55.49 | 133 |
| * | DMR_176 | chr5 | 140835903 | 140836925 | 1022 | 8.00E−62 | 48.76 | 118 |
| | DMR_177 | chr11 | 34438475 | 34439567 | 1092 | 1.70E−61 | 76.37 | 78 |
| * | DMR_178 | chr5 | 140787811 | 140788408 | 597 | 1.80E−61 | 49.2 | 128 |
| * | DMR_179 | chr12 | 63821720 | 63822267 | 547 | 2.40E−61 | 75.53 | 80 |
| | DMR_180 | chr1 | 147077098 | 147078590 | 1492 | 2.60E−61 | 48.57 | 149 |
| * | DMR_182 | chr21 | 46161319 | 46161947 | 628 | 5.90E−61 | 61.52 | 74 |
| * | DMR_183 | chr5 | 140862619 | 140863334 | 715 | 8.10E−61 | 53.62 | 102 |
| | DMR_184 | chr19 | 3404345 | 3405274 | 929 | 1.30E−60 | 78.22 | 82 |
| | DMR_185 | chr5 | 141224803 | 141225732 | 929 | 1.60E−60 | 56.63 | 117 |
| | DMR_186 | chr7 | 128240697 | 128241298 | 601 | 2.00E−60 | 69.46 | 88 |
| | DMR_187 | chr6 | 27282872 | 27284088 | 1216 | 3.70E−60 | 63.47 | 103 |
| | DMR_190 | chr2 | 120576671 | 120577260 | 589 | 6.10E−60 | 74.89 | 85 |
| | DMR_191 | chr14 | 93231208 | 93232331 | 1123 | 1.10E−59 | 64.91 | 86 |
| | DMR_192 | chr1 | 40132666 | 40133339 | 673 | 2.70E−59 | 70.37 | 88 |
| * | DMR_193 | chr2 | 25161002 | 25161766 | 764 | 3.00E−59 | 60.34 | 86 |
| | DMR_194 | chr16 | 53372943 | 53373683 | 740 | 3.10E−59 | 72.99 | 86 |
| | DMR_195 | chr19 | 2290614 | 2292171 | 1557 | 4.10E−59 | 47.38 | 210 |
| | DMR_196 | chr4 | 1404919 | 1405506 | 587 | 6.20E−59 | 67.61 | 96 |
| | DMR_197 | chr7 | 63900271 | 63901336 | 1065 | 8.40E−59 | 59.34 | 116 |
| * | DMR_198 | chr19 | 53992935 | 53993334 | 399 | 1.20E−58 | 65.9 | 78 |
| | DMR_199 | chr8 | 8702421 | 8703148 | 727 | 1.20E−58 | 51.76 | 105 |
| | DMR_200 | chrX | 101291092 | 101291532 | 440 | 1.50E−58 | 54.7 | 111 |
| | DMR_201 | chr8 | 48514400 | 48515351 | 951 | 1.80E−58 | 87.43 | 67 |
| | DMR_202 | chr14 | 73591729 | 73592574 | 845 | 2.00E−58 | 47.87 | 173 |
| * | DMR_203 | chr11 | 75428083 | 75428910 | 827 | 2.40E−58 | 76.66 | 66 |
| * | DMR_204 | chr7 | 158996827 | 158997425 | 598 | 3.70E−58 | 61.46 | 102 |
| | DMR_205 | chr1 | 143661084 | 143662512 | 1428 | 6.00E−58 | 61.71 | 108 |
| | DMR_206 | chr19 | 22807208 | 22807806 | 598 | 8.70E−58 | 73.92 | 85 |
| | DMR_207 | chr12 | 1907633 | 1908345 | 712 | 1.30E−57 | 70.64 | 85 |
| * | DMR_208 | chr11 | 396756 | 397535 | 779 | 1.40E−57 | 68.59 | 85 |
| | DMR_209 | chr21 | 36069806 | 36070679 | 873 | 1.40E−57 | 48.57 | 117 |
| * | DMR_210 | chr6 | 3849699 | 3850403 | 704 | 1.70E−57 | 49.23 | 91 |
| | DMR_211 | chr2 | 232386379 | 232387832 | 1453 | 2.10E−57 | 47.3 | 209 |
| | DMR_212 | chr14 | 103102562 | 103103090 | 528 | 3.40E−57 | 66.78 | 87 |
| * | DMR_213 | chr5 | 140876281 | 140877101 | 820 | 4.10E−57 | 51.42 | 98 |
| | DMR_214 | chr1 | 229431511 | 229433253 | 1742 | 4.40E−57 | 45.77 | 217 |
| | DMR_215 | chr11 | 2166415 | 2167014 | 599 | 5.40E−57 | 68.51 | 95 |
| | DMR_217 | chr3 | 13204113 | 13204712 | 599 | 6.40E−57 | 65.17 | 99 |
| | DMR_218 | chr17 | 8199849 | 8200650 | 801 | 9.60E−57 | 80.97 | 69 |
| | DMR_219 | chr18 | 12896886 | 12897154 | 268 | 1.10E−56 | 92.71 | 59 |
| * | DMR_220 | chr1 | 121116707 | 121117975 | 1268 | 1.30E−56 | 75.05 | 80 |
| | DMR_221 | chr3 | 42264974 | 42265911 | 937 | 2.40E−56 | 71.2 | 86 |
| * | DMR_222 | chr5 | 140877115 | 140877371 | 256 | 4.10E−56 | 68.75 | 66 |
| | DMR_223 | chr13 | 113129832 | 113130377 | 545 | 9.10E−56 | 61.46 | 85 |
| * | DMR_224 | chr5 | 140802516 | 140802825 | 309 | 1.10E−55 | 52.56 | 85 |
| | DMR_226 | chr19 | 14473224 | 14473788 | 564 | 1.20E−55 | 86.03 | 58 |
| | DMR_227 | chr8 | 144700058 | 144700495 | 437 | 1.40E−55 | 80.13 | 77 |
| | DMR_228 | chr4 | 1402272 | 1403174 | 902 | 2.10E−55 | 58.66 | 114 |
| | DMR_229 | chr20 | 45097916 | 45098375 | 459 | 3.60E−55 | 69.14 | 85 |
| * | DMR_230 | chr1 | 228158048 | 228158685 | 637 | 4.40E−55 | 50.25 | 107 |
| | DMR_231 | chr10 | 43350690 | 43351177 | 487 | 5.50E−55 | 69.78 | 76 |
| | DMR_232 | chr3 | 133746117 | 133746576 | 459 | 5.90E−55 | 70.79 | 81 |
| * | DMR_233 | chr5 | 141235852 | 141236420 | 568 | 6.90E−55 | 59.66 | 96 |
| | DMR_234 | chr17 | 78040210 | 78041482 | 1272 | 7.00E−55 | 75.08 | 77 |
| * | DMR_235 | chr5 | 42924036 | 42924453 | 417 | 7.30E−55 | 47.39 | 74 |
| | DMR_236 | chr6 | 16216253 | 16217006 | 753 | 7.90E−55 | 70.62 | 75 |
| * | DMR_238 | chr11 | 420275 | 420759 | 484 | 8.90E−55 | 68.03 | 72 |
| | DMR_239 | chr7 | 289651 | 290909 | 1258 | 1.50E−54 | 53.98 | 151 |
| | DMR_240 | chr1 | 244460827 | 244461592 | 765 | 2.40E−54 | 38.27 | 96 |
| | DMR_241 | chr17 | 4899951 | 4900701 | 750 | 3.10E−54 | 58.7 | 97 |
| | DMR_242 | chr21 | 37220114 | 37221705 | 1591 | 3.10E−54 | 49.74 | 185 |
| * | DMR_243 | chr16 | 1534498 | 1536296 | 1798 | 4.20E−54 | 51.54 | 128 |
| * | DMR_244 | chr11 | 6570888 | 6571363 | 475 | 5.20E−54 | 53.97 | 86 |
| * | DMR_245 | chr5 | 178165573 | 178165866 | 293 | 6.40E−54 | 85.51 | 62 |
| * | DMR_246 | chr19 | 36309259 | 36310115 | 856 | 7.90E−54 | 46.53 | 118 |
| | DMR_247 | chr19 | 2252117 | 2252776 | 659 | 8.10E−54 | 70.31 | 85 |
| | DMR_249 | chr7 | 35971406 | 35971796 | 390 | 9.20E−54 | 61.62 | 81 |
| | DMR_250 | chr18 | 13136310 | 13137285 | 975 | 1.10E−53 | 56.1 | 118 |
| | DMR_252 | chr11 | 62443995 | 62445738 | 1743 | 1.70E−53 | 66.2 | 86 |
| * | DMR_253 | chr9 | 96924791 | 96925345 | 554 | 1.80E−53 | 55.7 | 71 |
| * | DMR_254 | chr17 | 20896120 | 20896658 | 538 | 1.90E−53 | 56.32 | 68 |
| * | DMR_255 | chr18 | 79638272 | 79638865 | 593 | 2.40E−53 | 59.89 | 93 |
| | DMR_256 | chr20 | 3660222 | 3661595 | 1373 | 2.90E−53 | 50.36 | 173 |
| | DMR_257 | chr16 | 19114863 | 19115365 | 502 | 3.50E−53 | 73.23 | 76 |
| | DMR_259 | chr2 | 26562368 | 26563463 | 1095 | 3.70E−53 | 53.96 | 141 |
| * | DMR_260 | chr6 | 112367152 | 112367499 | 347 | 4.00E−53 | 67.19 | 70 |
| | DMR_261 | chr22 | 46081207 | 46082084 | 877 | 4.30E−53 | 79.6 | 68 |
| | DMR_262 | chr3 | 48656738 | 48657169 | 431 | 5.80E−53 | 71.53 | 78 |
| * | DMR_263 | chr7 | 67364373 | 67364971 | 598 | 1.10E−52 | 45.27 | 104 |
| | DMR_264 | chr20 | 60087403 | 60088030 | 627 | 1.40E−52 | 72.79 | 74 |
| * | DMR_265 | chr5 | 140672397 | 140672978 | 581 | 1.40E−52 | 70.23 | 61 |
| * | DMR_266 | chr7 | 50400052 | 50400636 | 584 | 1.40E−52 | 67.2 | 82 |
| | DMR_267 | chr6 | 24357152 | 24358454 | 1302 | 2.20E−52 | 57.95 | 118 |
| | DMR_268 | chr2 | 238163287 | 238164468 | 1181 | 2.30E−52 | 54.7 | 147 |
| | DMR_269 | chr9 | 120894399 | 120895033 | 634 | 3.60E−52 | 60 | 102 |
| * | DMR_270 | chr2 | 237410882 | 237415032 | 4150 | 6.20E−52 | 54.42 | 157 |
| | DMR_271 | chr9 | 96877416 | 96877701 | 285 | 6.40E−52 | 84.29 | 58 |
| | DMR_272 | chr19 | 12513087 | 12514019 | 932 | 1.20E−51 | 71.15 | 74 |
| | DMR_273 | chr2 | 232351015 | 232351326 | 311 | 2.10E−51 | 82.71 | 62 |
| | DMR_274 | chr2 | 636398 | 636993 | 595 | 2.60E−51 | 56.86 | 92 |
| * | DMR_275 | chr8 | 22086563 | 22087012 | 449 | 3.40E−51 | 65.41 | 68 |
| | DMR_276 | chr19 | 51948912 | 51949499 | 587 | 4.00E−51 | 66.89 | 74 |
| | DMR_277 | chr14 | 77641633 | 77642337 | 704 | 6.80E−51 | 75.38 | 75 |
| * | DMR_278 | chr5 | 28927708 | 28928240 | 532 | 8.10E−51 | 61.07 | 84 |
| * | DMR_279 | chr13 | 48318653 | 48319176 | 523 | 9.00E−51 | 46.97 | 73 |
| * | DMR_280 | chr5 | 141303749 | 141304240 | 491 | 9.20E−51 | 59.68 | 71 |
| | DMR_281 | chr13 | 43022908 | 43024412 | 1504 | 1.30E−50 | 50.01 | 121 |
| | DMR_282 | chr3 | 13932789 | 13933333 | 544 | 1.30E−50 | 76.81 | 66 |
| | DMR_283 | chr1 | 17149183 | 17149668 | 485 | 3.20E−50 | 78.83 | 57 |
| | DMR_284 | chrX | 100407437 | 100408006 | 569 | 3.20E−50 | 47.8 | 106 |
| | DMR_285 | chr4 | 11368697 | 11369234 | 537 | 8.00E−50 | 64.86 | 64 |
| * | DMR_286 | chr4 | 131975273 | 131976230 | 957 | 1.20E−49 | 39.08 | 108 |
| * | DMR_287 | chr6 | 87122559 | 87123075 | 516 | 1.30E−49 | 58.5 | 96 |
| | DMR_288 | chr1 | 143904645 | 143906096 | 1451 | 1.60E−49 | 58.41 | 82 |
| | DMR_289 | chr8 | 141205450 | 141206453 | 1003 | 1.70E−49 | 54.61 | 119 |
| | DMR_290 | chr18 | 15197652 | 15198310 | 658 | 2.20E−49 | 73.99 | 79 |
| * | DMR_291 | chr19 | 56840049 | 56840767 | 718 | 2.30E−49 | 63.81 | 80 |
| | DMR_292 | chr5 | 141476980 | 141478541 | 1561 | 2.30E−49 | 52.89 | 136 |
| * | DMR_293 | chr2 | 230990553 | 230990906 | 353 | 3.70E−49 | 71.82 | 61 |
| * | DMR_294 | chr5 | 157690356 | 157690893 | 537 | 4.00E−49 | 64.89 | 60 |
| | DMR_295 | chr21 | 29018553 | 29019561 | 1008 | 4.80E−49 | 39.45 | 92 |
| | DMR_296 | chr11 | 62687896 | 62688272 | 376 | 5.20E−49 | 69.23 | 77 |
| | DMR_297 | chr2 | 230827983 | 230828828 | 845 | 5.40E−49 | 61.36 | 96 |
| | DMR_298 | chr17 | 5115761 | 5116721 | 960 | 7.80E−49 | 59.7 | 93 |
| * | DMR_299 | chr1 | 146393088 | 146394036 | 948 | 1.40E−48 | 61.57 | 84 |
| * | DMR_300 | chr9 | 35035980 | 35036738 | 758 | 1.40E−48 | 64.48 | 70 |
| * | DMR_301 | chr17 | 38840518 | 38841636 | 1118 | 2.20E−48 | 43.63 | 94 |
| * | DMR_302 | chr5 | 140850277 | 140850540 | 263 | 2.70E−48 | 61.18 | 59 |
| | DMR_303 | chr9 | 136845215 | 136846003 | 788 | 2.70E−48 | 76.86 | 57 |
| | DMR_304 | chr10 | 96089724 | 96090338 | 614 | 3.40E−48 | 76.79 | 66 |
| * | DMR_305 | chr20 | 44750048 | 44750659 | 611 | 4.20E−48 | 44.18 | 118 |
| | DMR_306 | chr11 | 125886864 | 125887453 | 589 | 8.70E−48 | 80.36 | 61 |
| | DMR_307 | chr5 | 1245802 | 1246430 | 628 | 1.00E−47 | 73.52 | 65 |
| | DMR_308 | chr12 | 124517083 | 124518076 | 993 | 1.20E−47 | 68.31 | 84 |
| | DMR_309 | chr11 | 85682410 | 85683081 | 671 | 1.30E−47 | 69.66 | 72 |
| * | DMR_310 | chr16 | 15144891 | 15146257 | 1366 | 1.40E−47 | 50.3 | 100 |
| * | DMR_311 | chr5 | 141351867 | 141352395 | 528 | 1.60E−47 | 60.73 | 80 |
| | DMR_312 | chr19 | 55639989 | 55640520 | 531 | 1.90E−47 | 71.32 | 69 |
| | DMR_313 | chr18 | 12911937 | 12912223 | 286 | 2.00E−47 | 67.44 | 64 |
| * | DMR_314 | chr2 | 131646566 | 131647323 | 757 | 2.00E−47 | 61.8 | 88 |
| | DMR_315 | chr10 | 35607476 | 35608352 | 876 | 3.50E−47 | 78.98 | 60 |
| | DMR_316 | chr3 | 194397374 | 194398794 | 1420 | 3.70E−47 | 48.88 | 135 |
| * | DMR_317 | chr14 | 104155263 | 104155929 | 666 | 4.60E−47 | 78.01 | 61 |
| | DMR_318 | chr2 | 176066814 | 176069023 | 2209 | 5.80E−47 | 38.45 | 221 |
| | DMR_319 | chr10 | 42242145 | 42242858 | 713 | 6.60E−47 | 57.95 | 92 |
| | DMR_320 | chr16 | 19885236 | 19886231 | 995 | 7.50E−47 | 65.36 | 89 |
| * | DMR_322 | chr2 | 130229080 | 130229559 | 479 | 1.20E−46 | 52.21 | 89 |
| | DMR_323 | chr19 | 10514184 | 10514895 | 711 | 1.50E−46 | 43.35 | 149 |
| | DMR_324 | chr22 | 50546636 | 50547626 | 990 | 1.50E−46 | 61.02 | 94 |
| | DMR_325 | chr3 | 129045831 | 129046214 | 383 | 1.80E−46 | 77.89 | 60 |
| * | DMR_326 | chr19 | 39891784 | 39892146 | 362 | 2.30E−46 | 54.59 | 81 |
| | DMR_327 | chr10 | 89837217 | 89837885 | 668 | 2.80E−46 | 86.25 | 61 |
| | DMR_328 | chr2 | 95526153 | 95527324 | 1171 | 3.60E−46 | 46.37 | 169 |
| * | DMR_329 | chr15 | 82298504 | 82298858 | 354 | 4.10E−46 | 76.91 | 57 |
| | DMR_330 | chr1 | 157194799 | 157195344 | 545 | 4.90E−46 | 57.13 | 99 |
| * | DMR_331 | chr17 | 50781168 | 50781576 | 408 | 5.20E−46 | 58.97 | 76 |
| | DMR_332 | chr19 | 17872266 | 17873199 | 933 | 6.00E−46 | 62.92 | 93 |
| | DMR_333 | chr18 | 50298321 | 50298881 | 560 | 7.10E−46 | 71.33 | 64 |
| | DMR_334 | chr16 | 1160093 | 1160869 | 776 | 7.90E−46 | 53.07 | 109 |
| | DMR_335 | chr5 | 42949263 | 42950452 | 1189 | 7.90E−46 | 61.39 | 76 |
| * | DMR_336 | chr2 | 88284014 | 88284470 | 456 | 8.60E−46 | 63.22 | 77 |
| * | DMR_337 | chr5 | 141174430 | 141174953 | 523 | 9.20E−46 | 61.19 | 68 |
| * | DMR_338 | chr5 | 140842538 | 140842939 | 401 | 1.20E−45 | 58.41 | 62 |
| * | DMR_339 | chr4 | 54149468 | 54149760 | 292 | 1.30E−45 | 59.59 | 57 |
| * | DMR_340 | chrX | 1466354 | 1466679 | 325 | 1.30E−45 | 87.43 | 54 |
| * | DMR_341 | chr8 | 143727743 | 143728163 | 420 | 1.40E−45 | 70.79 | 52 |
| | DMR_343 | chr9 | 117744813 | 117745515 | 702 | 2.90E−45 | 55.17 | 96 |
| * | DMR_344 | chr21 | 39385615 | 39385952 | 337 | 3.20E−45 | 63.12 | 53 |
| | DMR_345 | chr1 | 143643014 | 143643456 | 442 | 3.30E−45 | 74.99 | 67 |
| | DMR_346 | chr17 | 74356688 | 74357435 | 747 | 4.00E−45 | 46.07 | 152 |
| | DMR_347 | chr14 | 24316308 | 24316873 | 565 | 6.90E−45 | 58.52 | 94 |
| | DMR_348 | chr11 | 280880 | 281298 | 418 | 1.00E−44 | 71.88 | 65 |
| | DMR_349 | chr6 | 32095617 | 32096340 | 723 | 1.10E−44 | 49.64 | 92 |
| | DMR_350 | chr1 | 182952748 | 182953055 | 307 | 1.30E−44 | 83.25 | 50 |
| | DMR_351 | chr19 | 37855169 | 37855520 | 351 | 1.30E−44 | 82.07 | 55 |
| | DMR_352 | chr4 | 55793461 | 55794287 | 826 | 1.40E−44 | 60.59 | 93 |
| | DMR_353 | chr9 | 96719547 | 96720110 | 563 | 1.50E−44 | 73.89 | 65 |
| | DMR_354 | chr22 | 20670771 | 20671419 | 648 | 2.60E−44 | 73.31 | 64 |
| * | DMR_355 | chr5 | 141236426 | 141237190 | 764 | 2.70E−44 | 66.49 | 65 |
| * | DMR_356 | chr1 | 148328866 | 148329783 | 917 | 2.80E−44 | 44.88 | 102 |
| * | DMR_357 | chr1 | 205849533 | 205850522 | 989 | 2.80E−44 | 55.48 | 75 |
| | DMR_358 | chr15 | 90664973 | 90666051 | 1078 | 3.90E−44 | 47.45 | 152 |
| * | DMR_359 | chr5 | 141123659 | 141124222 | 563 | 3.90E−44 | 66.19 | 60 |
| | DMR_360 | chr12 | 52789848 | 52790494 | 646 | 4.10E−44 | 63.42 | 76 |
| | DMR_361 | chr1 | 44738213 | 44739091 | 878 | 4.20E−44 | 56.68 | 68 |
| * | DMR_362 | chr1 | 228158690 | 228159027 | 337 | 4.20E−44 | 67.74 | 56 |
| * | DMR_363 | chr16 | 33028208 | 33029285 | 1077 | 4.20E−44 | 52.49 | 102 |
| | DMR_364 | chr19 | 23470897 | 23471550 | 653 | 4.60E−44 | 69.9 | 67 |
| | DMR_365 | chr1 | 178486610 | 178487234 | 624 | 5.30E−44 | 78.49 | 57 |
| | DMR_366 | chr20 | 25853583 | 25854223 | 640 | 6.00E−44 | 73.3 | 66 |
| | DMR_367 | chr1 | 28774889 | 28775799 | 910 | 6.80E−44 | 65.49 | 82 |
| | DMR_368 | chr8 | 124972900 | 124973848 | 948 | 7.10E−44 | 65.54 | 75 |
| | DMR_369 | chr18 | 37274552 | 37275034 | 482 | 7.70E−44 | 82.77 | 60 |
| * | DMR_370 | chr9 | 41355744 | 41357023 | 1279 | 9.20E−44 | 69.07 | 77 |
| | DMR_371 | chr17 | 62138038 | 62138707 | 669 | 1.10E−43 | 80.42 | 58 |
| * | DMR_372 | chr22 | 36563599 | 36564756 | 1157 | 1.20E−43 | 55.95 | 86 |
| * | DMR_373 | chr8 | 73285618 | 73285974 | 356 | 1.20E−43 | 63.94 | 60 |
| * | DMR_374 | chr2 | 96500447 | 96500887 | 440 | 1.30E−43 | 72.46 | 64 |
| | DMR_375 | chr7 | 66043644 | 66044765 | 1121 | 1.60E−43 | 60.64 | 95 |
| * | DMR_376 | chr17 | 74919808 | 74920496 | 688 | 2.00E−43 | 42.33 | 115 |
| | DMR_377 | chr2 | 233938835 | 233939457 | 622 | 2.50E−43 | 67.14 | 69 |
| | DMR_378 | chr1 | 22953673 | 22953886 | 213 | 2.60E−43 | 83.86 | 54 |
| * | DMR_380 | chr17 | 19979174 | 19980541 | 1367 | 2.80E−43 | 53.51 | 69 |
| * | DMR_381 | chr9 | 137417237 | 137417891 | 654 | 3.40E−43 | 68.37 | 46 |
| | DMR_382 | chr1 | 35120731 | 35121270 | 539 | 3.70E−43 | 75.36 | 58 |
| | DMR_383 | chr11 | 281298 | 281668 | 370 | 4.40E−43 | 74.79 | 57 |
| | DMR_384 | chr9 | 39809229 | 39810157 | 928 | 5.00E−43 | 41.61 | 103 |
| * | DMR_385 | chr18 | 80160136 | 80160414 | 278 | 6.90E−43 | 62.36 | 48 |
| * | DMR_386 | chr6 | 134115321 | 134115812 | 491 | 7.40E−43 | 65.72 | 62 |
| * | DMR_387 | chr19 | 22517609 | 22518041 | 432 | 7.60E−43 | 60.28 | 66 |
| * | DMR_388 | chr5 | 141188991 | 141189380 | 389 | 9.60E−43 | 64.41 | 64 |
| * | DMR_389 | chr5 | 141123308 | 141123604 | 296 | 1.10E−42 | 64.27 | 57 |
| | DMR_390 | chr2 | 24402783 | 24403379 | 596 | 1.30E−42 | 70.3 | 63 |
| | DMR_391 | chrX | 15675450 | 15676803 | 1353 | 1.30E−42 | 28.52 | 97 |
| * | DMR_392 | chr12 | 80716861 | 80717337 | 476 | 1.80E−42 | 58.88 | 64 |
| * | DMR_393 | chr5 | 140787522 | 140787780 | 258 | 2.10E−42 | 53.34 | 66 |
| * | DMR_394 | chr11 | 129618086 | 129618408 | 322 | 2.30E−42 | 74.43 | 54 |
| * | DMR_395 | chr2 | 132256542 | 132257064 | 522 | 2.70E−42 | 78.14 | 59 |
| | DMR_396 | chr6 | 44275737 | 44276083 | 346 | 2.80E−42 | 80.91 | 57 |
| | DMR_397 | chr14 | 35825192 | 35825732 | 540 | 3.30E−42 | 67.89 | 57 |
| | DMR_398 | chr19 | 38791266 | 38792507 | 1241 | 3.30E−42 | 59.21 | 97 |
| | DMR_399 | chr4 | 183721667 | 183722430 | 763 | 3.50E−42 | 60.18 | 59 |
| * | DMR_400 | chr7 | 140435476 | 140435820 | 344 | 4.00E−42 | 52.85 | 73 |
| * | DMR_401 | chr7 | 55449184 | 55449579 | 395 | 4.40E−42 | 73.17 | 61 |
| | DMR_402 | chr7 | 128271380 | 128271915 | 535 | 6.00E−42 | 75 | 61 |
| * | DMR_404 | chr6 | 89887497 | 89888065 | 568 | 6.70E−42 | 64.97 | 67 |
| | DMR_405 | chr1 | 108660751 | 108661755 | 1004 | 6.90E−42 | 46.14 | 171 |
| | DMR_406 | chr2 | 231483020 | 231484052 | 1032 | 7.70E−42 | 50.81 | 103 |
| | DMR_407 | chr16 | 88938709 | 88940113 | 1404 | 7.80E−42 | 60.07 | 88 |
| | DMR_408 | chr8 | 142700184 | 142700783 | 599 | 8.00E−42 | 41.2 | 96 |
| * | DMR_409 | chr11 | 78196165 | 78196621 | 456 | 8.10E−42 | 50.26 | 63 |
| | DMR_410 | chr1 | 2207452 | 2208219 | 767 | 8.50E−42 | 73.96 | 63 |
| * | DMR_411 | chr16 | 1533946 | 1534483 | 537 | 8.50E−42 | 74.07 | 64 |
| | DMR_412 | chr13 | 113109643 | 113110109 | 466 | 8.60E−42 | 72.23 | 65 |
| * | DMR_413 | chrX | 2324971 | 2325816 | 845 | 9.70E−42 | 73.01 | 55 |
| | DMR_415 | chr22 | 50603240 | 50604340 | 1100 | 1.20E−41 | 44.23 | 151 |
| * | DMR_416 | chr19 | 37059142 | 37059483 | 341 | 2.10E−41 | 55.67 | 65 |
| * | DMR_417 | chr18 | 61554250 | 61554637 | 387 | 2.50E−41 | 34.02 | 86 |
| | DMR_418 | chr9 | 128192192 | 128193362 | 1170 | 2.70E−41 | 52.38 | 109 |
| * | DMR_419 | chr10 | 133446000 | 133446724 | 724 | 3.20E−41 | 50.09 | 69 |
| | DMR_420 | chr5 | 141364008 | 141364937 | 929 | 3.40E−41 | 58.64 | 78 |
| | DMR_421 | chr1 | 223393380 | 223393744 | 364 | 3.60E−41 | 68.48 | 64 |
| * | DMR_422 | chr16 | 71441540 | 71441981 | 441 | 3.60E−41 | 69.54 | 57 |
| | DMR_423 | chr1 | 1290830 | 1291864 | 1034 | 3.80E−41 | 55.6 | 92 |
| * | DMR_424 | chr17 | 20784271 | 20784669 | 398 | 3.90E−41 | 67.87 | 63 |
| | DMR_425 | chr15 | 34495584 | 34495918 | 334 | 4.80E−41 | 79.36 | 49 |
| | DMR_426 | chr19 | 615950 | 616845 | 895 | 4.80E−41 | 65.26 | 72 |
| | DMR_427 | chr20 | 45316292 | 45317015 | 723 | 4.90E−41 | 67.97 | 64 |
| | DMR_428 | chr22 | 30921989 | 30922458 | 469 | 5.50E−41 | 63.52 | 64 |
| | DMR_429 | chr3 | 40476957 | 40477921 | 964 | 6.20E−41 | 24.45 | 80 |
| | DMR_430 | chr11 | 278668 | 279619 | 951 | 6.50E−41 | 65.07 | 80 |
| | DMR_431 | chr13 | 113110109 | 113110894 | 785 | 7.30E−41 | 49.96 | 94 |
| | DMR_432 | chr2 | 102186829 | 102187672 | 843 | 9.10E−41 | 55.52 | 96 |
| | DMR_433 | chr9 | 131289880 | 131290838 | 958 | 1.00E−40 | 58.39 | 80 |
| | DMR_434 | chr6 | 28259232 | 28259620 | 388 | 1.20E−40 | 68.66 | 63 |
| | DMR_435 | chr14 | 105364152 | 105364616 | 464 | 1.30E−40 | 87.99 | 45 |
| * | DMR_436 | chr10 | 122149580 | 122150056 | 476 | 1.40E−40 | 74.3 | 50 |
| * | DMR_437 | chr16 | 87216864 | 87217337 | 473 | 1.50E−40 | 68.33 | 68 |
| | DMR_439 | chr2 | 91589318 | 91589888 | 570 | 1.90E−40 | 46.59 | 103 |
| | DMR_440 | chr5 | 54518934 | 54520265 | 1331 | 2.00E−40 | 57.29 | 97 |
| | DMR_441 | chr4 | 385880 | 387129 | 1249 | 2.10E−40 | 55.06 | 100 |
| | DMR_442 | chr7 | 153886098 | 153886995 | 897 | 2.40E−40 | 67.42 | 68 |
| * | DMR_443 | chr18 | 79863915 | 79864111 | 196 | 2.60E−40 | 63.65 | 46 |
| * | DMR_444 | chr10 | 50024783 | 50025171 | 388 | 2.90E−40 | 77.3 | 54 |
| | DMR_445 | chr15 | 30224960 | 30226120 | 1160 | 3.10E−40 | 48.9 | 137 |
| | DMR_446 | chr8 | 97277311 | 97278283 | 972 | 3.10E−40 | 46.86 | 152 |
| | DMR_447 | chr12 | 43758309 | 43759667 | 1358 | 3.30E−40 | 46.08 | 138 |
| * | DMR_448 | chr14 | 100828008 | 100828287 | 279 | 3.30E−40 | 69.79 | 42 |
| | DMR_449 | chr19 | 17393877 | 17394310 | 433 | 3.40E−40 | 68.98 | 62 |
| | DMR_450 | chr14 | 64540729 | 64541012 | 283 | 3.80E−40 | 77.09 | 56 |
| | DMR_451 | chr5 | 33938119 | 33939446 | 1327 | 3.80E−40 | 52.97 | 92 |
| * | DMR_452 | chr19 | 53554195 | 53554689 | 494 | 5.00E−40 | 76.99 | 42 |
| * | DMR_453 | chr4 | 1311131 | 1311739 | 608 | 5.20E−40 | 39.94 | 71 |
| | DMR_454 | chr19 | 3097222 | 3098434 | 1212 | 5.40E−40 | 62.67 | 69 |
| * | DMR_455 | chr16 | 46844412 | 46844609 | 197 | 5.90E−40 | 71.65 | 52 |
| | DMR_456 | chr6 | 37625005 | 37625860 | 855 | 6.40E−40 | 74.18 | 60 |
| * | DMR_457 | chr18 | 59969080 | 59969941 | 861 | 8.60E−40 | 64.53 | 61 |
| | DMR_458 | chr3 | 46898409 | 46899026 | 617 | 1.20E−39 | 68.27 | 60 |
| | DMR_460 | chr5 | 140800945 | 140801683 | 738 | 1.90E−39 | 62.79 | 70 |
| | DMR_461 | chr1 | 143651717 | 143652738 | 1021 | 2.10E−39 | 49.95 | 103 |
| | DMR_462 | chr19 | 11848762 | 11849369 | 607 | 2.20E−39 | 66.47 | 70 |
| | DMR_463 | chr6 | 73523459 | 73523906 | 447 | 2.30E−39 | 49.63 | 77 |
| * | DMR_464 | chr19 | 19514315 | 19514696 | 381 | 2.90E−39 | 83.08 | 38 |
| | DMR_465 | chr16 | 30474028 | 30474587 | 559 | 3.30E−39 | 69.44 | 62 |
| * | DMR_466 | chr8 | 53711918 | 53712805 | 887 | 3.30E−39 | 58.39 | 52 |
| | DMR_467 | chr22 | 46073812 | 46074794 | 982 | 3.40E−39 | 63.41 | 63 |
| * | DMR_469 | chr5 | 140829169 | 140829542 | 373 | 3.80E−39 | 48.21 | 74 |
| | DMR_470 | chr14 | 37610882 | 37611628 | 746 | 4.00E−39 | 51.82 | 91 |
| | DMR_471 | chr6 | 101398726 | 101399861 | 1135 | 4.30E−39 | 48.08 | 106 |
| | DMR_472 | chr19 | 47545268 | 47545645 | 377 | 4.50E−39 | 68.37 | 58 |
| * | DMR_473 | chr17 | 81498999 | 81499413 | 414 | 5.60E−39 | 53.18 | 62 |
| * | DMR_474 | chr13 | 20394444 | 20395014 | 570 | 6.10E−39 | 54.66 | 76 |
| * | DMR_475 | chr2 | 74436051 | 74436763 | 712 | 6.60E−39 | 52.29 | 65 |
| | DMR_476 | chr9 | 83956133 | 83957207 | 1074 | 6.70E−39 | 48.78 | 124 |
| | DMR_477 | chr7 | 63926230 | 63926716 | 486 | 7.20E−39 | 70.91 | 64 |
| | DMR_478 | chr16 | 2967435 | 2967770 | 335 | 7.40E−39 | 64.43 | 66 |
| | DMR_479 | chr6 | 73461627 | 73462135 | 508 | 7.40E−39 | 23.65 | 90 |
| * | DMR_480 | chr22 | 20424819 | 20425714 | 895 | 7.60E−39 | 42.62 | 111 |
| | DMR_481 | chr10 | 122879518 | 122880174 | 656 | 8.00E−39 | 46.38 | 128 |
| * | DMR_482 | chr2 | 149320248 | 149320911 | 663 | 8.30E−39 | 50.5 | 77 |
| | DMR_483 | chr16 | 6483019 | 6483383 | 364 | 9.90E−39 | 51.25 | 93 |
| | DMR_484 | chr19 | 1325045 | 1325317 | 272 | 1.10E−38 | 82.88 | 51 |
| | DMR_485 | chr17 | 62138707 | 62139190 | 483 | 1.40E−38 | 55.76 | 66 |
| | DMR_486 | chr1 | 228276332 | 228276832 | 500 | 1.50E−38 | 60.52 | 75 |
| * | DMR_487 | chr15 | 78830813 | 78831337 | 524 | 1.60E−38 | 60.41 | 77 |
| | DMR_488 | chr17 | 19579586 | 19580513 | 927 | 1.70E−38 | 52.61 | 114 |
| | DMR_489 | chr2 | 110211678 | 110212853 | 1175 | 1.70E−38 | 51.26 | 89 |
| | DMR_490 | chr1 | 201283778 | 201284304 | 526 | 1.90E−38 | 60.73 | 75 |
| * | DMR_491 | chr11 | 72012929 | 72014405 | 1476 | 1.90E−38 | 44.85 | 100 |
| * | DMR_492 | chr5 | 141371888 | 141372309 | 421 | 1.90E−38 | 60.13 | 64 |
| | DMR_493 | chr11 | 61777257 | 61777894 | 637 | 2.00E−38 | 57.24 | 76 |
| | DMR_494 | chr1 | 156245443 | 156246290 | 847 | 2.10E−38 | 51.37 | 126 |
| * | DMR_495 | chr19 | 13702559 | 13702738 | 179 | 3.30E−38 | 88.7 | 38 |
| | DMR_496 | chr6 | 142088166 | 142088848 | 682 | 3.40E−38 | 55.57 | 85 |
| | DMR_497 | chr17 | 4786024 | 4787088 | 1064 | 4.00E−38 | 59.7 | 75 |
| * | DMR_498 | chr3 | 48210582 | 48210896 | 314 | 5.10E−38 | 59.06 | 58 |
| * | DMR_499 | chr13 | 23945706 | 23946578 | 872 | 5.20E−38 | 42.95 | 71 |
| * | DMR_500 | chr7 | 2762614 | 2763578 | 964 | 5.20E−38 | 69.17 | 53 |
| * | DMR_501 | chr10 | 133465164 | 133465643 | 479 | 5.30E−38 | 49.83 | 91 |
| | DMR_502 | chr19 | 50050157 | 50051428 | 1271 | 5.50E−38 | 51.09 | 110 |
| | DMR_504 | chr22 | 18091644 | 18092607 | 963 | 6.20E−38 | 77.29 | 54 |
| | DMR_505 | chr17 | 74357437 | 74357659 | 222 | 6.60E−38 | 69.71 | 56 |
| * | DMR_506 | chr3 | 112637972 | 112639929 | 1957 | 6.60E−38 | 58.73 | 90 |
| * | DMR_507 | chr1 | 2300712 | 2301539 | 827 | 7.10E−38 | 65.68 | 71 |
| | DMR_508 | chr2 | 164955099 | 164955685 | 586 | 8.00E−38 | 73.67 | 55 |
| | DMR_509 | chr3 | 49199209 | 49199823 | 614 | 8.30E−38 | 78.38 | 51 |
| | DMR_510 | chr17 | 72640294 | 72640987 | 693 | 8.50E−38 | 64.13 | 62 |
| | DMR_511 | chr5 | 137888941 | 137889756 | 815 | 9.20E−38 | 50.26 | 105 |
| | DMR_512 | chr11 | 126414460 | 126416896 | 2436 | 1.10E−37 | 51.57 | 107 |
| | DMR_513 | chr17 | 75077258 | 75078141 | 883 | 1.10E−37 | 50.39 | 125 |
| * | DMR_514 | chr6 | 29014917 | 29015208 | 291 | 1.10E−37 | 62.39 | 54 |
| | DMR_515 | chr12 | 75207436 | 75208045 | 609 | 1.20E−37 | 46.5 | 135 |
| | DMR_516 | chr22 | 16602522 | 16603290 | 768 | 1.30E−37 | 64.61 | 65 |
| | DMR_517 | chr16 | 4375631 | 4378556 | 2925 | 1.70E−37 | 42.72 | 163 |
| | DMR_518 | chr15 | 56732131 | 56733952 | 1821 | 1.80E−37 | 48.5 | 113 |
| | DMR_519 | chr17 | 19724532 | 19724984 | 452 | 2.80E−37 | 71.07 | 59 |
| * | DMR_520 | chr14 | 103928275 | 103928461 | 186 | 3.40E−37 | 71.23 | 45 |
| * | DMR_521 | chr19 | 2634248 | 2635114 | 866 | 3.50E−37 | 57.83 | 71 |
| * | DMR_522 | chr5 | 140843132 | 140844451 | 1319 | 3.50E−37 | 44.06 | 95 |
| | DMR_523 | chr19 | 48480190 | 48481432 | 1242 | 3.70E−37 | 46.4 | 143 |
| | DMR_524 | chr2 | 74415060 | 74415469 | 409 | 3.80E−37 | 68.44 | 68 |
| * | DMR_525 | chr22 | 36323400 | 36325667 | 2267 | 3.80E−37 | 46.23 | 112 |
| | DMR_526 | chr9 | 121700466 | 121701433 | 967 | 4.10E−37 | 55.14 | 76 |
| | DMR_527 | chr8 | 144524504 | 144524692 | 188 | 4.70E−37 | 62.18 | 51 |
| | DMR_528 | chr16 | 3028761 | 3029953 | 1192 | 5.60E−37 | 49.62 | 127 |
| | DMR_529 | chr19 | 2249925 | 2250693 | 768 | 6.00E−37 | 62.07 | 66 |
| * | DMR_530 | chr9 | 39132934 | 39133261 | 327 | 6.10E−37 | 66.85 | 57 |
| | DMR_531 | chr1 | 145486952 | 145487930 | 978 | 6.20E−37 | 51.23 | 92 |
| | DMR_532 | chr1 | 2322586 | 2323013 | 427 | 6.80E−37 | 73.86 | 52 |
| * | DMR_533 | chr16 | 28747084 | 28747478 | 394 | 6.80E−37 | 78.96 | 48 |
| | DMR_534 | chr1 | 143698344 | 143700189 | 1845 | 7.20E−37 | 48.53 | 100 |
| * | DMR_535 | chr19 | 6238699 | 6239704 | 1005 | 8.60E−37 | 63.44 | 71 |
| | DMR_536 | chr20 | 56003842 | 56004362 | 520 | 9.40E−37 | 46.18 | 93 |
| * | DMR_537 | chr5 | 141373964 | 141374385 | 421 | 1.10E−36 | 62.64 | 61 |
| * | DMR_538 | chr19 | 35309476 | 35310096 | 620 | 1.30E−36 | 50.86 | 79 |
| | DMR_539 | chr18 | 3411908 | 3412244 | 336 | 1.40E−36 | 71.36 | 53 |
| | DMR_540 | chr22 | 41998555 | 41999090 | 535 | 1.40E−36 | 73.08 | 48 |
| * | DMR_541 | chr19 | 58367669 | 58368061 | 392 | 1.50E−36 | 53.12 | 55 |
| | DMR_542 | chr10 | 133527569 | 133528094 | 525 | 1.70E−36 | 51.76 | 71 |
| | DMR_543 | chr1 | 16758938 | 16759956 | 1018 | 1.80E−36 | 39.11 | 124 |
| | DMR_544 | chr14 | 95714069 | 95714634 | 565 | 1.90E−36 | 60.39 | 73 |
| | DMR_545 | chr19 | 50358474 | 50358722 | 248 | 2.20E−36 | 85.43 | 48 |
| | DMR_546 | chr18 | 14450119 | 14450469 | 350 | 2.30E−36 | 65.88 | 66 |
| | DMR_547 | chr4 | 14862839 | 14863263 | 424 | 2.30E−36 | 63.4 | 70 |
| * | DMR_548 | chr7 | 768376 | 769113 | 737 | 2.70E−36 | 50.59 | 70 |
| | DMR_551 | chr3 | 142947357 | 142947701 | 344 | 3.50E−36 | 64.31 | 48 |
| * | DMR_552 | chr5 | 141241296 | 141241658 | 362 | 3.70E−36 | 60.12 | 69 |
| * | DMR_553 | chr5 | 141441862 | 141442121 | 259 | 3.70E−36 | 70.25 | 42 |
| | DMR_554 | chr6 | 73451886 | 73452458 | 572 | 4.70E−36 | 29.37 | 96 |
| * | DMR_555 | chr19 | 5102981 | 5103659 | 678 | 5.00E−36 | 64.62 | 47 |
| * | DMR_556 | chr16 | 34161895 | 34162761 | 866 | 5.20E−36 | 40.63 | 93 |
| | DMR_557 | chr6 | 149450744 | 149451757 | 1013 | 5.60E−36 | 44.56 | 157 |
| | DMR_558 | chr17 | 34637765 | 34638036 | 271 | 5.80E−36 | 76 | 49 |
| | DMR_559 | chr2 | 53859742 | 53860206 | 464 | 5.80E−36 | 57.21 | 81 |
| * | DMR_560 | chr3 | 139020661 | 139021080 | 419 | 6.80E−36 | 43.1 | 80 |
| * | DMR_561 | chr10 | 125773778 | 125774004 | 226 | 6.90E−36 | 67.02 | 42 |
| * | DMR_562 | chr19 | 12555431 | 12555723 | 292 | 6.90E−36 | 64.45 | 63 |
| | DMR_563 | chr19 | 23203849 | 23204510 | 661 | 7.30E−36 | 62.07 | 67 |
| | DMR_564 | chr16 | 3500131 | 3500401 | 270 | 9.30E−36 | 80.34 | 47 |
| | DMR_565 | chr3 | 87088619 | 87089912 | 1293 | 9.90E−36 | 51.03 | 101 |
| | DMR_566 | chr12 | 4911361 | 4911813 | 452 | l.00E−35 | 52.24 | 71 |
| * | DMR_567 | chr2 | 10006384 | 10006802 | 418 | l.00E−35 | 69.81 | 48 |
| | DMR_568 | chr2 | 94735635 | 94735891 | 256 | 1.20E−35 | 67.15 | 61 |
| | DMR_569 | chrX | 13653068 | 13653470 | 402 | 1.20E−35 | 30.55 | 71 |
| | DMR_570 | chr1 | 143904069 | 143904645 | 576 | 1.40E−35 | 84.16 | 45 |
| | DMR_571 | chr11 | 1874320 | 1876526 | 2206 | 1.40E−35 | 48.92 | 113 |
| * | DMR_572 | chr15 | 65077123 | 65077598 | 475 | 1.60E−35 | 41.84 | 82 |
| | DMR_574 | chr1 | 227786396 | 227787111 | 715 | 2.00E−35 | 59.08 | 69 |
| | DMR_575 | chr20 | 19975077 | 19976333 | 1256 | 2.00E−35 | 57.21 | 79 |
| * | DMR_576 | chrS | 141194287 | 141194743 | 456 | 2.00E−35 | 52.72 | 71 |
| * | DMR_577 | chr17 | 42040078 | 42040425 | 347 | 2.30E−35 | 67.2 | 51 |
| | DMR_578 | chr12 | 65881723 | 65882806 | 1083 | 2.50E−35 | 66.6 | 59 |
| * | DMR_579 | chr10 | 79274306 | 79275615 | 1309 | 2.60E−35 | 51.93 | 88 |
| | DMR_580 | chr16 | 20348516 | 20348806 | 290 | 2.70E−35 | 71.2 | 52 |
| | DMR_581 | chr6 | 73309604 | 73310324 | 720 | 2.80E−35 | 45.07 | 117 |
| | DMR_582 | chr18 | 50267861 | 50268572 | 711 | 2.90E−35 | 64.66 | 65 |
| | DMR_583 | chr19 | 37251255 | 37252127 | 872 | 3.00E−35 | 60.09 | 67 |
| | DMR_584 | chr7 | 5072003 | 5072800 | 797 | 3.00E−35 | 82.98 | 47 |
| | DMR_585 | chr19 | 10513826 | 10514179 | 353 | 3.10E−35 | 63.28 | 58 |
| | DMR_586 | chr1 | 207669312 | 207669933 | 621 | 3.30E−35 | 50.32 | 98 |
| | DMR_588 | chr1 | 217924860 | 217925323 | 463 | 4.30E−35 | 56.17 | 70 |
| | DMR_589 | chr6 | 57961158 | 57961724 | 566 | 6.00E−35 | 32.04 | 52 |
| | DMR_590 | chr5 | 176596592 | 176597676 | 1084 | 6.70E−35 | 35.94 | 206 |
| | DMR_591 | chr5 | 141382661 | 141383287 | 626 | 7.90E−35 | 61.92 | 66 |
| | DMR_592 | chr10 | 79982758 | 79983835 | 1077 | 8.40E−35 | 66.14 | 58 |
| | DMR_593 | chr12 | 52601169 | 52601481 | 312 | 9.20E−35 | 68.61 | 62 |
| * | DMR_594 | chr22 | 46089234 | 46090564 | 1330 | 9.40E−35 | 51.68 | 88 |
| | DMR_595 | chr15 | 31483105 | 31483657 | 552 | 9.50E−35 | 57.44 | 83 |
| | DMR_596 | chr8 | 38650740 | 38651182 | 442 | 9.80E−35 | 54.63 | 93 |
| | DMR_597 | chr19 | 9497854 | 9498594 | 740 | 1.10E−34 | 54.9 | 73 |
| | DMR_598 | chr19 | 48444049 | 48444828 | 779 | 1.10E−34 | 65.15 | 58 |
| | DMR_599 | chr2 | 208406425 | 208407453 | 1028 | 1.10E−34 | 42.75 | 145 |
| * | DMR_600 | chr9 | 137416835 | 137417230 | 395 | 1.10E−34 | 41.18 | 52 |
| | DMR_601 | chr19 | 40600152 | 40600794 | 642 | 1.20E−34 | 68.28 | 62 |
| | DMR_603 | chr9 | 136844071 | 136844746 | 675 | 1.30E−34 | 35.33 | 151 |
| | DMR_604 | chr2 | 226797423 | 226798365 | 942 | 1.40E−34 | 41.74 | 95 |
| * | DMR_605 | chr12 | 9450446 | 9450753 | 307 | 1.60E−34 | 47.59 | 42 |
| | DMR_606 | chr1 | 157978735 | 157979294 | 559 | 1.70E−34 | 63.84 | 65 |
| | DMR_607 | chr11 | 66567943 | 66568946 | 1003 | 1.80E−34 | 46.1 | 137 |
| | DMR_608 | chr11 | 47590279 | 47590490 | 211 | 1.90E−34 | 61.6 | 55 |
| * | DMR_609 | chr19 | 6220170 | 6221277 | 1107 | 2.00E−34 | 54.89 | 79 |
| * | DMR_610 | chr3 | 128653319 | 128654249 | 930 | 2.00E−34 | 54.95 | 59 |
| * | DMR_611 | chr3 | 28200367 | 28200789 | 422 | 2.10E−34 | 47.99 | 68 |
| | DMR_612 | chr4 | 15702722 | 15703377 | 655 | 2.20E−34 | 45.4 | 71 |
| | DMR_613 | chr1 | 19273921 | 19274291 | 370 | 2.50E−34 | 68.52 | 56 |
| | DMR_614 | chr4 | 1194307 | 1194998 | 691 | 2.50E−34 | 63.49 | 78 |
| * | DMR_615 | chr19 | 58217108 | 58217362 | 254 | 2.60E−34 | 61.66 | 46 |
| | DMR_616 | chr19 | 56765174 | 56765719 | 545 | 2.80E−34 | 69.82 | 59 |
| | DMR_617 | chr7 | 5397048 | 5397646 | 598 | 2.80E−34 | 65.75 | 51 |
| | DMR_618 | chr2 | 114662309 | 114662639 | 330 | 2.90E−34 | 55.79 | 72 |
| * | DMR_619 | chr1 | 85615997 | 85616432 | 435 | 3.00E−34 | 55.54 | 51 |
| | DMR_620 | chr5 | 79069774 | 79070407 | 633 | 3.00E−34 | 59.93 | 75 |
| | DMR_622 | chr4 | 86903971 | 86904771 | 800 | 3.30E−34 | 66.4 | 70 |
| * | DMR_623 | chr8 | 143726883 | 143727361 | 478 | 3.40E−34 | 37.37 | 77 |
| * | DMR_624 | chr5 | 140877374 | 140877946 | 572 | 3.50E−34 | 52.65 | 55 |
| | DMR_625 | chr10 | 42177515 | 42178135 | 620 | 3.60E−34 | 78.15 | 45 |
| * | DMR_626 | chr5 | 140802964 | 140803315 | 351 | 3.80E−34 | 51.29 | 60 |
| | DMR_627 | chr17 | 8002877 | 8003427 | 550 | 3.90E−34 | 48.77 | 76 |
| * | DMR_628 | chr5 | 179632478 | 179633092 | 614 | 3.90E−34 | 38.8 | 55 |
| | DMR_629 | chr20 | 59031680 | 59032296 | 616 | 4.10E−34 | 26.27 | 82 |
| | DMR_630 | chr9 | 138147649 | 138148158 | 509 | 5.10E−34 | 57.52 | 73 |
| | DMR_631 | chr19 | 57105992 | 57107080 | 1088 | 5.30E−34 | 48.65 | 101 |
| * | DMR_632 | chr7 | 72975942 | 72976274 | 332 | 6.00E−34 | 59.29 | 42 |
| * | DMR_633 | chr6 | 88047633 | 88048041 | 408 | 6.20E−34 | 46.14 | 76 |
| | DMR_634 | chr22 | 19941143 | 19942188 | 1045 | 6.70E−34 | 57.6 | 60 |
| | DMR_635 | chr5 | 140827875 | 140828430 | 555 | 6.70E−34 | 64.07 | 62 |
| * | DMR_636 | chr19 | 47097476 | 47098308 | 832 | 8.10E−34 | 61.12 | 64 |
| | DMR_637 | chr2 | 3703101 | 3703946 | 845 | 8.10E−34 | 50.11 | 119 |
| | DMR_638 | chr7 | 128270807 | 128271226 | 419 | 8.30E−34 | 69.71 | 54 |
| * | DMR_639 | chr2 | 26728106 | 26728392 | 286 | 9.30E−34 | 70.24 | 38 |
| | DMR_640 | chr19 | 51887310 | 51888537 | 1227 | 9.70E−34 | 48.18 | 114 |
| | DMR_641 | chr20 | 3673295 | 3673793 | 498 | 9.70E−34 | 54.36 | 58 |
| | DMR_642 | chrX | 53224838 | 53225282 | 444 | 9.80E−34 | 28.99 | 80 |
| * | DMR_643 | chr17 | 58487556 | 58488216 | 660 | 1.00E−33 | 50.83 | 75 |
| * | DMR_644 | chr5 | 140857451 | 140857860 | 409 | l.00E−33 | 53.6 | 55 |
| * | DMR_645 | chr10 | 30818866 | 30819919 | 1053 | 1.10E−33 | 63.66 | 56 |
| | DMR_646 | chr17 | 18634673 | 18635401 | 728 | 1.10E−33 | 45.49 | 118 |
| * | DMR_647 | chr19 | 612957 | 613305 | 348 | 1.10E−33 | 42.5 | 55 |
| | DMR_648 | chr11 | 128824189 | 128824481 | 292 | 1.20E−33 | 76.87 | 49 |
| | DMR_650 | chr19 | 36009212 | 36009822 | 610 | 1.40E−33 | 47.88 | 92 |
| * | DMR_651 | chr5 | 141215469 | 141215960 | 491 | 1.40E−33 | 50.66 | 77 |
| | DMR_652 | chr19 | 9283438 | 9283981 | 543 | 1.50E−33 | 58.94 | 72 |
| | DMR_653 | chr9 | 136364154 | 136364476 | 322 | 1.70E−33 | 66.8 | 54 |
| * | DMR_654 | chr7 | 48847924 | 48848214 | 290 | 1.80E−33 | 55.19 | 63 |
| | DMR_655 | chr1 | 6455087 | 6455459 | 372 | 2.20E−33 | 53.81 | 91 |
| | DMR_656 | chr2 | 131829705 | 131830287 | 582 | 2.40E−33 | 39.69 | 129 |
| | DMR_657 | chr2 | 120912146 | 120912968 | 822 | 2.50E−33 | 73.91 | 49 |
| | DMR_658 | chr5 | 141402709 | 141403253 | 544 | 2.70E−33 | 65.1 | 57 |
| | DMR_659 | chr18 | 14999280 | 14999545 | 265 | 2.90E−33 | 64.28 | 52 |
| * | DMR_661 | chrX | 100406925 | 100407350 | 425 | 3.10E−33 | 45.36 | 68 |
| * | DMR_662 | chr11 | 1868254 | 1870222 | 1968 | 3.20E−33 | 49.35 | 114 |
| * | DMR_663 | chr18 | 80160414 | 80160610 | 196 | 3.40E−33 | 51.45 | 48 |
| | DMR_664 | chr2 | 224442576 | 224442991 | 415 | 3.40E−33 | 73.41 | 52 |
| | DMR_665 | chr10 | 38093820 | 38094382 | 562 | 3.80E−33 | 42.42 | 66 |
| | DMR_666 | chr11 | 88508600 | 88509488 | 888 | 3.80E−33 | 42.89 | 149 |
| | DMR_667 | chr18 | 79507376 | 79507840 | 464 | 4.00E−33 | 78.84 | 49 |
| | DMR_668 | chr20 | 20003029 | 20004364 | 1335 | 4.50E−33 | 61.65 | 57 |
| | DMR_669 | chr6 | 17580243 | 17580950 | 707 | 4.60E−33 | 64.68 | 56 |
| | DMR_670 | chr19 | 19110111 | 19111097 | 986 | 4.80E−33 | 44.44 | 127 |
| * | DMR_671 | chr11 | 3231922 | 3232931 | 1009 | 4.90E−33 | 45.53 | 98 |
| | DMR_672 | chr1 | 152515218 | 152515874 | 656 | 5.00E−33 | 55.39 | 74 |
| * | DMR_673 | chr12 | 6132469 | 6132840 | 371 | 5.30E−33 | 46.99 | 48 |
| | DMR_674 | chr19 | 22009906 | 22010965 | 1059 | 5.60E−33 | 53.18 | 81 |
| * | DMR_675 | chr4 | 26804488 | 26805118 | 630 | 6.20E−33 | 37.51 | 64 |
| * | DMR_676 | chr2 | 236214372 | 236214535 | 163 | 7.20E−33 | 78.15 | 41 |
| * | DMR_678 | chr3 | 120306597 | 120306874 | 277 | 7.60E−33 | 62.72 | 45 |
| | DMR_679 | chr5 | 8457028 | 8458397 | 1369 | 7.80E−33 | 37.36 | 139 |
| | DMR_680 | chr6 | 28090906 | 28091431 | 525 | 9.00E−33 | 70.08 | 55 |
| | DMR_681 | chr12 | 101209533 | 101210359 | 826 | 9.10E−33 | 40.48 | 146 |
| | DMR_683 | chr6 | 158516147 | 158516525 | 378 | 9.70E−33 | 58.66 | 68 |
| * | DMR_684 | chr11 | 2909595 | 2909883 | 288 | 1.00E−32 | 76.63 | 43 |
| * | DMR_685 | chr7 | 152366749 | 152367241 | 492 | 1.00E−32 | 60.24 | 48 |
| * | DMR_686 | chr11 | 128451345 | 128452198 | 853 | 1.10E−32 | 60.2 | 72 |
| | DMR_687 | chr5 | 191357 | 191885 | 528 | 1.10E−32 | 70.52 | 56 |
| | DMR_688 | chr6 | 170023190 | 170023595 | 405 | 1.10E−32 | 71.11 | 47 |
| | DMR_689 | chr14 | 103126680 | 103127460 | 780 | 1.20E−32 | 50.9 | 101 |
| | DMR_690 | chr19 | 22532253 | 22533234 | 981 | 1.60E−32 | 56.08 | 72 |
| | DMR_691 | chr19 | 36797050 | 36797791 | 741 | 1.80E−32 | 45.75 | 117 |
| * | DMR_692 | chr2 | 130036887 | 130037419 | 532 | 1.80E−32 | 42.34 | 74 |
| * | DMR_693 | chr7 | 4265015 | 4265430 | 415 | 1.90E−32 | 47.24 | 70 |
| | DMR_694 | chr1 | 203075628 | 203075983 | 355 | 2.00E−32 | 69.21 | 49 |
| | DMR_695 | chr19 | 22427768 | 22428282 | 514 | 2.00E−32 | 61.62 | 59 |
| | DMR_697 | chr20 | 45970522 | 45971513 | 991 | 3.20E−32 | 71.74 | 54 |
| * | DMR_698 | chr16 | 88405499 | 88406694 | 1195 | 3.90E−32 | 65.98 | 58 |
| | DMR_700 | chr22 | 46080441 | 46080918 | 477 | 5.00E−32 | 80.74 | 39 |
| | DMR_701 | chr11 | 1871079 | 1873829 | 2750 | 5.10E−32 | 46.23 | 111 |
| | DMR_702 | chr2 | 94857833 | 94858511 | 678 | 5.10E−32 | 60.02 | 67 |
| * | DMR_703 | chr6 | 116564997 | 116565523 | 526 | 5.10E−32 | 63.73 | 52 |
| | DMR_705 | chr3 | 39502023 | 39502690 | 667 | 6.20E−32 | 58.04 | 57 |
| | DMR_706 | chr7 | 66505989 | 66506679 | 690 | 7.20E−32 | 70.66 | 43 |
| * | DMR_707 | chr11 | 60852872 | 60853137 | 265 | 7.30E−32 | 51.98 | 43 |
| | DMR_708 | chr9 | 136846447 | 136846818 | 371 | 7.60E−32 | 29.43 | 96 |
| | DMR_709 | chr19 | 38770344 | 38771169 | 825 | 8.20E−32 | 59.74 | 57 |
| | DMR_710 | chr3 | 42905770 | 42906871 | 1101 | 9.80E−32 | 44.37 | 121 |
| | DMR_711 | chr16 | 28623453 | 28623665 | 212 | 1.10E−31 | 83.09 | 43 |
| | DMR_713 | chr4 | 3041143 | 3041784 | 641 | 1.10E−31 | 50.37 | 78 |
| | DMR_714 | chr6 | 167796478 | 167796839 | 361 | 1.10E−31 | 79.71 | 44 |
| | DMR_715 | chr10 | 52778039 | 52779169 | 1130 | 1.20E−31 | 47.53 | 86 |
| * | DMR_716 | chr19 | 53554703 | 53555186 | 483 | 1.20E−31 | 66.69 | 42 |
| | DMR_718 | chr19 | 45385349 | 45386784 | 1435 | 1.30E−31 | 52.25 | 89 |
| * | DMR_719 | chr9 | 32955593 | 32956065 | 472 | 1.30E−31 | 57.29 | 45 |
| * | DMR_720 | chr9 | 38526669 | 38526937 | 268 | 1.30E−31 | 65.28 | 51 |
| | DMR_721 | chr9 | 122225835 | 122226346 | 511 | 1.40E−31 | 44.41 | 100 |
| | DMR_722 | chr16 | 2785483 | 2785734 | 251 | 1.50E−31 | 69.05 | 53 |
| | DMR_724 | chr6 | 117547876 | 117548367 | 491 | 1.50E−31 | 59.4 | 66 |
| * | DMR_725 | chr2 | 1652746 | 1653972 | 1226 | 1.60E−31 | 69.73 | 51 |
| | DMR_726 | chr22 | 38081772 | 38082875 | 1103 | 1.60E−31 | 50.38 | 87 |
| * | DMR_728 | chr11 | 18455482 | 18456263 | 781 | 1.90E−31 | 42.63 | 63 |
| | DMR_729 | chr9 | 120842525 | 120843331 | 806 | 1.90E−31 | 47.05 | 110 |
| | DMR_731 | chr19 | 58439640 | 58440634 | 994 | 2.50E−31 | 37.75 | 97 |
| | DMR_732 | chr3 | 125958251 | 125958732 | 481 | 2.70E−31 | 53.61 | 84 |
| * | DMR_733 | chr17 | 78997800 | 78999007 | 1207 | 2.80E−31 | 51.97 | 71 |
| | DMR_734 | chr19 | 47003891 | 47004435 | 544 | 2.80E−31 | 77.98 | 45 |
| | DMR_735 | chr20 | 62433626 | 62434649 | 1023 | 3.00E−31 | 52.92 | 75 |
| | DMR_736 | chr1 | 179591555 | 179592070 | 515 | 3.40E−31 | 59.26 | 69 |
| | DMR_737 | chr22 | 45889614 | 45890093 | 479 | 3.80E−31 | 66.37 | 53 |
| | DMR_738 | chr10 | 75043674 | 75044070 | 396 | 4.60E−31 | 53.3 | 63 |
| * | DMR_739 | chr5 | 141393356 | 141394390 | 1034 | 5.20E−31 | 50.34 | 59 |
| * | DMR_741 | chrX | 21656188 | 21656948 | 760 | 5.40E−31 | 48.61 | 58 |
| | DMR_743 | chr3 | 130747382 | 130748035 | 653 | 5.80E−31 | 74.58 | 45 |
| * | DMR_744 | chr22 | 46090928 | 46092315 | 1387 | 5.90E−31 | 47 | 67 |
| | DMR_745 | chr19 | 39407846 | 39408747 | 901 | 6.00E−31 | 56.94 | 78 |
| * | DMR_746 | chr11 | 118971669 | 118971913 | 244 | 6.50E−31 | 69.73 | 42 |
| * | DMR_747 | chr11 | 77137930 | 77138547 | 617 | 6.90E−31 | 73.41 | 44 |
| | DMR_748 | chr17 | 45144199 | 45145020 | 821 | 7.00E−31 | 55.3 | 83 |
| | DMR_749 | chr10 | 89251457 | 89252107 | 650 | 7.40E−31 | 16.71 | 97 |
| | DMR_750 | chr19 | 14474055 | 14474267 | 212 | 7.80E−31 | 73.29 | 41 |
| | DMR_751 | chr2 | 55282210 | 55282584 | 374 | 8.10E−31 | 26.61 | 86 |
| | DMR_752 | chr7 | 155257227 | 155258109 | 882 | 8.20E−31 | 63.82 | 56 |
| * | DMR_753 | chr8 | 22275279 | 22275556 | 277 | 8.40E−31 | 55.14 | 43 |
| * | DMR_754 | chr7 | 767520 | 768037 | 517 | 8.60E−31 | 51.04 | 45 |
| * | DMR_755 | chr6 | 85114071 | 85114594 | 523 | 9.10E−31 | 72.17 | 42 |
| | DMR_756 | chr10 | 64041475 | 64041938 | 463 | 9.40E−31 | 60.6 | 58 |
| * | DMR_757 | chr19 | 46022712 | 46023393 | 681 | 9.50E−31 | 70.46 | 51 |
| | DMR_758 | chr21 | 45454989 | 45456205 | 1216 | 9.60E−31 | 56.52 | 84 |
| | DMR_759 | chr14 | 69571535 | 69572001 | 466 | 9.80E−31 | 50.27 | 82 |
| | DMR_760 | chr2 | 74414444 | 74415060 | 616 | 1.00E−30 | 57.56 | 71 |
| * | DMR_761 | chr13 | 22696580 | 22696773 | 193 | 1.10E−30 | 52.04 | 53 |
| | DMR_762 | chr14 | 64541013 | 64541523 | 510 | 1.10E−30 | 53.1 | 53 |
| | DMR_763 | chr4 | 52750837 | 52751716 | 879 | 1.10E−30 | 44.55 | 128 |
| * | DMR_764 | chr5 | 141427535 | 141427862 | 327 | 1.20E−30 | 58.24 | 46 |
| * | DMR_766 | chr5 | 141247426 | 141247666 | 240 | 1.50E−30 | 57.87 | 45 |
| * | DMR_767 | chr8 | 23070815 | 23071438 | 623 | 1.50E−30 | 59.51 | 52 |
| | DMR_768 | chr3 | 8767850 | 8768535 | 685 | 1.60E−30 | 49.99 | 100 |
| * | DMR_769 | chr19 | 13070271 | 13070958 | 687 | 1.70E−30 | 41.83 | 67 |
| * | DMR_770 | chr13 | 46438018 | 46438305 | 287 | 1.80E−30 | 53.99 | 53 |
| * | DMR_771 | chr9 | 41953037 | 41953508 | 471 | 1.80E−30 | 46.04 | 90 |
| | DMR_772 | chr19 | 55433145 | 55433716 | 571 | 1.90E−30 | 60.07 | 69 |
| | DMR_773 | chr13 | 24328322 | 24328515 | 193 | 2.30E−30 | 77.61 | 43 |
| | DMR_775 | chr6 | 31728149 | 31728953 | 804 | 2.90E−30 | 49.02 | 78 |
| | DMR_776 | chr21 | 42685011 | 42685657 | 646 | 3.00E−30 | 46.01 | 76 |
| * | DMR_777 | chr5 | 140871150 | 140871332 | 182 | 3.00E−30 | 69.58 | 40 |
| * | DMR_778 | chr9 | 136361085 | 136361499 | 414 | 3.00E−30 | 61.92 | 48 |
| * | DMR_779 | chr2 | 117859422 | 117859888 | 466 | 3.10E−30 | 41.74 | 52 |
| * | DMR_781 | chr2 | 237886098 | 237887064 | 966 | 3.20E−30 | 44.19 | 78 |
| * | DMR_782 | chr14 | 64601560 | 64602081 | 521 | 3.30E−30 | 72.7 | 37 |
| | DMR_783 | chr19 | 50458535 | 50459064 | 529 | 3.60E−30 | 60.47 | 58 |
| * | DMR_784 | chr1 | 211479026 | 211479386 | 360 | 3.70E−30 | 58.33 | 58 |
| * | DMR_785 | chr9 | 137368473 | 137369243 | 770 | 4.20E−30 | 57.63 | 56 |
| | DMR_786 | chr13 | 20141974 | 20142290 | 316 | 4.70E−30 | 75.97 | 40 |
| * | DMR_787 | chr1 | 161079583 | 161079947 | 364 | 5.00E−30 | 52.32 | 63 |
| * | DMR_788 | chr21 | 29076541 | 29078047 | 1506 | 5.00E−30 | 42.15 | 115 |
| * | DMR_789 | chr5 | 140883436 | 140883642 | 206 | 5.00E−30 | 62.17 | 41 |
| * | DMR_790 | chr10 | 47565733 | 47565996 | 263 | 5.50E−30 | 46.26 | 48 |
| | DMR_791 | chr1 | 23902604 | 23903403 | 799 | 5.60E−30 | 49.13 | 101 |
| * | DMR_792 | chr19 | 18849601 | 18851295 | 1694 | 6.60E−30 | 44.71 | 88 |
| | DMR_793 | chr2 | 106065281 | 106065944 | 663 | 6.60E−30 | 47.37 | 110 |
| * | DMR_794 | chr11 | 73957025 | 73957987 | 962 | 6.70E−30 | 56.9 | 61 |
| | DMR_795 | chr9 | 134441956 | 134442947 | 991 | 6.90E−30 | 61.38 | 60 |
| * | DMR_796 | chr14 | 103711458 | 103712817 | 1359 | 7.10E−30 | 53.2 | 70 |
| * | DMR_797 | chr5 | 140796641 | 140796956 | 315 | 7.40E−30 | 42.62 | 50 |
| | DMR_798 | chr19 | 1041818 | 1042412 | 594 | 7.90E−30 | 54.17 | 70 |
| | DMR_799 | chr3 | 146251415 | 146251941 | 526 | 7.90E−30 | 75.33 | 47 |
| | DMR_800 | chr17 | 35373971 | 35374941 | 970 | 8.40E−30 | 29.2 | 92 |
| | DMR_801 | chr9 | 42851510 | 42851921 | 411 | 8.40E−30 | 63.77 | 51 |
| * | DMR_802 | chr1 | 10639435 | 10639778 | 343 | 9.30E−30 | 58.79 | 49 |
| * | DMR_803 | chr2 | 240596331 | 240597065 | 734 | 9.30E−30 | 40.29 | 62 |
| | DMR_804 | chr7 | 23205942 | 23206337 | 395 | 9.50E−30 | 62.57 | 57 |
| * | DMR_805 | chr17 | 72342458 | 72343604 | 1146 | 1.00E−29 | 57.17 | 57 |
| | DMR_806 | chr5 | 42952924 | 42953522 | 598 | 1.10E−29 | 58.32 | 53 |
| | DMR_807 | chr16 | 1770167 | 1770729 | 562 | 1.20E−29 | 53.55 | 70 |
| * | DMR_808 | chr19 | 1009251 | 1009517 | 266 | 1.20E−29 | 61.05 | 37 |
| * | DMR_809 | chr2 | 23617383 | 23618348 | 965 | 1.20E−29 | 64.83 | 46 |
| | DMR_810 | chr19 | 2494816 | 2495337 | 521 | 1.40E−29 | 46.3 | 65 |
| * | DMR_811 | chr2 | 161340866 | 161341348 | 482 | 1.40E−29 | 61.94 | 36 |
| * | DMR_812 | chr6 | 18019769 | 18020142 | 373 | 1.40E−29 | 53.87 | 53 |
| | DMR_813 | chr16 | 3950290 | 3950968 | 678 | 1.60E−29 | 66.7 | 54 |
| * | DMR_814 | chr7 | 1076179 | 1077088 | 909 | 1.60E−29 | 60.54 | 58 |
| | DMR_815 | chr8 | 144713826 | 144714111 | 285 | 1.70E−29 | 56.66 | 59 |
| | DMR_816 | chr11 | 49208239 | 49208485 | 246 | 1.80E−29 | 79.73 | 43 |
| | DMR_817 | chr17 | 75036075 | 75037050 | 975 | 1.80E−29 | 63.58 | 57 |
| | DMR_818 | chr1 | 1237104 | 1238157 | 1053 | 2.00E−29 | 54.31 | 76 |
| | DMR_819 | chr7 | 963863 | 964900 | 1037 | 2.00E−29 | 65.71 | 59 |
| | DMR_820 | chr19 | 56643332 | 56643646 | 314 | 2.20E−29 | 71 | 47 |
| | DMR_821 | chr1 | 1238542 | 1239834 | 1292 | 2.40E−29 | 43.32 | 91 |
| * | DMR_822 | chr10 | 58326922 | 58327537 | 615 | 2.40E−29 | 59.57 | 63 |
| * | DMR_823 | chr1 | 146486599 | 146487661 | 1062 | 2.50E−29 | 44.07 | 110 |
| | DMR_824 | chr15 | 99507284 | 99508522 | 1238 | 2.70E−29 | 54.96 | 62 |
| * | DMR_825 | chr8 | 140100478 | 140100705 | 227 | 2.80E−29 | 60.14 | 36 |
| | DMR_826 | chr2 | 130692395 | 130693488 | 1093 | 2.90E−29 | 50.02 | 74 |
| | DMR_827 | chr9 | 137278779 | 137278935 | 156 | 2.90E−29 | 71.14 | 38 |
| * | DMR_828 | chr7 | 100381626 | 100382092 | 466 | 3.20E−29 | 47.18 | 51 |
| | DMR_830 | chr2 | 112431935 | 112432705 | 770 | 3.40E−29 | 79.54 | 38 |
| | DMR_831 | chr5 | 173232953 | 173233483 | 530 | 3.40E−29 | 38.37 | 85 |
| | DMR_833 | chr1 | 153789421 | 153791059 | 1638 | 3.60E−29 | 55.87 | 84 |
| * | DMR_834 | chr16 | 33059212 | 33059742 | 530 | 3.60E−29 | 61.89 | 46 |
| | DMR_835 | chr2 | 156320268 | 156321985 | 1717 | 3.70E−29 | 43.25 | 128 |
| * | DMR_836 | chr14 | 100826239 | 100826630 | 391 | 4.00E−29 | 63.37 | 37 |
| | DMR_838 | chr17 | 81536606 | 81536988 | 382 | 4.20E−29 | 72.23 | 48 |
| * | DMR_839 | chr11 | 14259047 | 14259661 | 614 | 4.40E−29 | 37.43 | 71 |
| * | DMR_840 | chr2 | 132257210 | 132257606 | 396 | 4.60E−29 | 82.41 | 42 |
| | DMR_841 | chr10 | 92592104 | 92592633 | 529 | 4.70E−29 | 62.74 | 46 |
| | DMR_842 | chr19 | 1154628 | 1155728 | 1100 | 4.70E−29 | 61.84 | 61 |
| | DMR_843 | chr3 | 131026450 | 131027049 | 599 | 4.70E−29 | 18.9 | 93 |
| | DMR_844 | chr6 | 28734044 | 28734685 | 641 | 5.00E−29 | 59.04 | 57 |
| | DMR_845 | chrX | 15674173 | 15675208 | 1035 | 5.30E−29 | 26.27 | 68 |
| * | DMR_846 | chr5 | 141241659 | 141242044 | 385 | 5.40E−29 | 55.08 | 63 |
| | DMR_847 | chr2 | 174729790 | 174730742 | 952 | 5.50E−29 | 46.96 | 105 |
| * | DMR_848 | chr7 | 64037260 | 64037808 | 548 | 5.60E−29 | 60.78 | 57 |
| * | DMR_849 | chr14 | 20723387 | 20723701 | 314 | 6.50E−29 | 71.43 | 46 |
| | DMR_850 | chr19 | 46078912 | 46079186 | 274 | 6.50E−29 | 55.69 | 67 |
| * | DMR_851 | chr19 | 37403407 | 37403762 | 355 | 6.60E−29 | 47.91 | 50 |
| * | DMR_852 | chr16 | 50673590 | 50673838 | 248 | 7.00E−29 | 66.15 | 42 |
| * | DMR_853 | chr10 | 103668126 | 103669232 | 1106 | 7.20E−29 | 59.44 | 62 |
| * | DMR_854 | chr5 | 141428145 | 141429155 | 1010 | 7.20E−29 | 43.4 | 63 |
| | DMR_857 | chr12 | 98745522 | 98746399 | 877 | 7.70E−29 | 47.83 | 112 |
| | DMR_858 | chr17 | 47778248 | 47778682 | 434 | 7.70E−29 | 60.38 | 60 |
| * | DMR_859 | chr10 | 27413463 | 27413721 | 258 | 8.00E−29 | 56.36 | 46 |
| | DMR_860 | chr6 | 81749042 | 81751474 | 2432 | 8.10E−29 | 57.87 | 58 |
| | DMR_861 | chr7 | 63925527 | 63926187 | 660 | 8.40E−29 | 57.87 | 60 |
| * | DMR_862 | chr16 | 2126438 | 2127200 | 762 | 8.50E−29 | 57.32 | 58 |
| | DMR_863 | chr11 | 65453984 | 65455063 | 1079 | 8.60E−29 | 57.34 | 73 |
| | DMR_864 | chr17 | 4901675 | 4902464 | 789 | 8.60E−29 | 44.04 | 69 |
| | DMR_865 | chr19 | 1456571 | 1457002 | 431 | 9.10E−29 | 68.99 | 43 |
| * | DMR_866 | chr16 | 34158859 | 34158990 | 131 | 9.20E−29 | 56.5 | 39 |
| | DMR_867 | chr12 | 125861377 | 125861939 | 562 | 9.90E−29 | 50.49 | 71 |
| | DMR_869 | chr12 | 116318122 | 116319603 | 1481 | 1.10E−28 | 39.59 | 97 |
| | DMR_871 | chr16 | 4680054 | 4681317 | 1263 | 1.20E−28 | 46.97 | 70 |
| | DMR_872 | chr21 | 41426011 | 41426332 | 321 | 1.20E−28 | 28.02 | 67 |
| | DMR_875 | chr7 | 23247125 | 23248231 | 1106 | 1.50E−28 | 49.75 | 73 |
| * | DMR_876 | chr16 | 3294890 | 3295238 | 348 | 1.70E−28 | 57.1 | 43 |
| | DMR_877 | chr3 | 194487357 | 194487870 | 513 | 1.80E−28 | 42.97 | 89 |
| * | DMR_878 | chr22 | 47154347 | 47155326 | 979 | 2.00E−28 | 58.65 | 69 |
| * | DMR_879 | chr16 | 81486482 | 81487885 | 1403 | 2.10E−28 | 54.7 | 62 |
| | DMR_880 | chr9 | 134403605 | 134404753 | 1148 | 2.10E−28 | 59.64 | 62 |
| * | DMR_881 | chr2 | 74130499 | 74130886 | 387 | 2.20E−28 | 60.24 | 52 |
| | DMR_882 | chr20 | 33719984 | 33720597 | 613 | 2.20E−28 | 47.04 | 85 |
| | DMR_883 | chr19 | 9362914 | 9363453 | 539 | 2.40E−28 | 66.41 | 55 |
| * | DMR_884 | chr19 | 49701199 | 49701745 | 546 | 2.70E−28 | 51.68 | 59 |
| * | DMR_885 | chr19 | 18306473 | 18307607 | 1134 | 3.00E−28 | 37.68 | 78 |
| | DMR_887 | chr2 | 240519382 | 240520689 | 1307 | 3.50E−28 | 45.77 | 110 |
| | DMR_888 | chr9 | 6715824 | 6716231 | 407 | 3.50E−28 | 51.22 | 72 |
| | DMR_889 | chr19 | 2272060 | 2273486 | 1426 | 3.80E−28 | 40.2 | 100 |
| * | DMR_890 | chr11 | 85934622 | 85935067 | 445 | 4.20E−28 | 66.15 | 43 |
| | DMR_891 | chr13 | 25300911 | 25301139 | 228 | 4.30E−28 | 81.14 | 35 |
| | DMR_892 | chr15 | 70473626 | 70475047 | 1421 | 4.50E−28 | 51.86 | 81 |
| | DMR_893 | chr17 | 15749563 | 15749824 | 261 | 4.60E−28 | 74.59 | 37 |
| | DMR_894 | chr19 | 13298665 | 13299045 | 380 | 4.70E−28 | 64.7 | 46 |
| * | DMR_895 | chr5 | 140884246 | 140884569 | 323 | 4.70E−28 | 49.26 | 49 |
| | DMR_896 | chr16 | 51134551 | 51135240 | 689 | 4.80E−28 | 54.59 | 76 |
| | DMR_897 | chr16 | 2966808 | 2967399 | 591 | 4.90E−28 | 46.47 | 90 |
| | DMR_898 | chr20 | 663825 | 664697 | 872 | 4.90E−28 | 48.2 | 99 |
| * | DMR_899 | chr18 | 79616635 | 79617078 | 443 | 5.10E−28 | 50.86 | 57 |
| * | DMR_900 | chr10 | 14508100 | 14509380 | 1280 | 5.40E−28 | 56.96 | 61 |
| | DMR_901 | chr2 | 127681110 | 127681825 | 715 | 5.50E−28 | 61.02 | 62 |
| * | DMR_902 | chr3 | 139020337 | 139020652 | 315 | 5.50E−28 | 45.19 | 50 |
| | DMR_903 | chr19 | 14209375 | 14209881 | 506 | 6.10E−28 | 28.32 | 89 |
| | DMR_904 | chr22 | 50548384 | 50549019 | 635 | 6.10E−28 | 81.12 | 43 |
| | DMR_906 | chr22 | 46066497 | 46067821 | 1324 | 6.30E−28 | 74.45 | 49 |
| * | DMR_907 | chr11 | 18209073 | 18209378 | 305 | 6.80E−28 | 71.83 | 44 |
| * | DMR_908 | chr21 | 43757639 | 43758565 | 926 | 7.10E−28 | 50.41 | 67 |
| * | DMR_909 | chr19 | 474274 | 475050 | 776 | 7.20E−28 | 42.02 | 88 |
| | DMR_910 | chr10 | 38402046 | 38402763 | 717 | 7.70E−28 | 41.95 | 92 |
| * | DMR_911 | chr12 | 100716089 | 100716506 | 417 | 8.20E−28 | 50.96 | 61 |
| * | DMR_912 | chr13 | 100975555 | 100976014 | 459 | 8.40E−28 | 53.46 | 42 |
| | DMR_913 | chr7 | 2717353 | 2717980 | 627 | 8.70E−28 | 56.9 | 60 |
| | DMR_914 | chr11 | 120169718 | 120170224 | 506 | 9.20E−28 | 62.73 | 50 |
| * | DMR_915 | chr10 | 103473263 | 103473698 | 435 | 9.70E−28 | 49.58 | 65 |
| * | DMR_916 | chr16 | 4319327 | 4320640 | 1313 | 1.00E−27 | 52.16 | 76 |
| | DMR_917 | chr2 | 207930661 | 207931135 | 474 | 1.00E−27 | 48.73 | 59 |
| | DMR_918 | chr5 | 141338922 | 141339688 | 766 | 1.00E−27 | 47.11 | 76 |
| | DMR_919 | chr7 | 100745135 | 100745563 | 428 | 1.00E−27 | 73.79 | 41 |
| | DMR_920 | chr19 | 15010828 | 15011331 | 503 | 1.10E−27 | 42.81 | 130 |
| | DMR_921 | chr4 | 1404069 | 1404490 | 421 | 1.10E−27 | 64.95 | 56 |
| | DMR_922 | chr2 | 130252573 | 130253165 | 592 | 1.20E−27 | 57.87 | 58 |
| | DMR_923 | chrX | 103220676 | 103221342 | 666 | 1.30E−27 | 44.61 | 62 |
| | DMR_924 | chr18 | 9138078 | 9139413 | 1335 | 1.40E−27 | 65.15 | 55 |
| | DMR_925 | chr9 | 94332304 | 94332916 | 612 | 1.40E−27 | 65.79 | 50 |
| | DMR_926 | chr1 | 152189260 | 152189474 | 214 | 1.60E−27 | 72.62 | 46 |
| * | DMR_927 | chr15 | 85749040 | 85750862 | 1822 | 1.60E−27 | 52.66 | 81 |
| * | DMR_928 | chr9 | 91159851 | 91160324 | 473 | 1.60E−27 | 67.68 | 43 |
| * | DMR_929 | chr1 | 40303358 | 40304260 | 902 | 1.70E−27 | 49.14 | 82 |
| * | DMR_931 | chr17 | 81277454 | 81278317 | 863 | 1.90E−27 | 39.66 | 55 |
| | DMR_933 | chr8 | 144713126 | 144713496 | 370 | 1.90E−27 | 63.34 | 54 |
| * | DMR_935 | chr19 | 13001621 | 13002061 | 440 | 2.10E−27 | 68.42 | 47 |
| | DMR_936 | chr11 | 47214157 | 47214725 | 568 | 2.30E−27 | 76.84 | 40 |
| | DMR_937 | chr3 | 138960188 | 138960983 | 795 | 2.30E−27 | 45.88 | 79 |
| * | DMR_938 | chr5 | 37208979 | 37209346 | 367 | 2.30E−27 | 55.09 | 35 |
| * | DMR_939 | chr7 | 74195216 | 74196541 | 1325 | 2.40E−27 | 51.15 | 64 |
| | DMR_940 | chr15 | 34495198 | 34495465 | 267 | 2.60E−27 | 71.4 | 43 |
| * | DMR_941 | chr16 | 67654431 | 67655042 | 611 | 2.60E−27 | 49.98 | 53 |
| | DMR_942 | chr2 | 197786185 | 197786361 | 176 | 2.90E−27 | 76.95 | 45 |
| | DMR_943 | chr17 | 58519209 | 58519773 | 564 | 3.10E−27 | 59.57 | 55 |
| | DMR_945 | chr17 | 4785219 | 4785455 | 236 | 3.50E−27 | 82.53 | 33 |
| * | DMR_946 | chr5 | 141366182 | 141366539 | 357 | 3.50E−27 | 64.79 | 44 |
| * | DMR_947 | chr19 | 5893850 | 5895045 | 1195 | 3.60E−27 | 53.13 | 62 |
| | DMR_948 | chr1 | 180953644 | 180954310 | 666 | 3.70E−27 | 76.97 | 43 |
| | DMR_949 | chr7 | 44064388 | 44065836 | 1448 | 3.70E−27 | 46 | 96 |
| | DMR_950 | chr17 | 4900803 | 4901131 | 328 | 3.80E−27 | 58.12 | 52 |
| | DMR_951 | chr16 | 88639543 | 88640039 | 496 | 3.90E−27 | 64.19 | 49 |
| * | DMR_952 | chr19 | 54434558 | 54435578 | 1020 | 3.90E−27 | 59.99 | 53 |
| | DMR_953 | chr19 | 10514896 | 10515070 | 174 | 4.00E−27 | 65.73 | 48 |
| | DMR_954 | chr11 | 503162 | 504160 | 998 | 4.10E−27 | 66.33 | 54 |
| * | DMR_955 | chr16 | 16150292 | 16150781 | 489 | 4.70E−27 | 70.26 | 46 |
| | DMR_957 | chr2 | 23663312 | 23663702 | 390 | 4.90E−27 | 73.48 | 42 |
| * | DMR_959 | chr7 | 57404636 | 57405071 | 435 | 4.90E−27 | 50.29 | 63 |
| * | DMR_960 | chr16 | 22424007 | 22425786 | 1779 | 5.20E−27 | 49.52 | 95 |
| | DMR_961 | chr9 | 62532273 | 62533389 | 1116 | 5.30E−27 | 34.49 | 66 |
| | DMR_962 | chr1 | 54781388 | 54781708 | 320 | 5.40E−27 | 41.3 | 71 |
| * | DMR_963 | chr1 | 2503648 | 2504478 | 830 | 5.50E−27 | 54.58 | 61 |
| | DMR_964 | chr15 | 77893695 | 77894831 | 1136 | 5.60E−27 | 50.05 | 72 |
| | DMR_965 | chr9 | 62897540 | 62898610 | 1070 | 5.70E−27 | 33.04 | 97 |
| | DMR_966 | chr11 | 72078642 | 72079690 | 1048 | 6.00E−27 | 55.49 | 61 |
| | DMR_967 | chr22 | 46083656 | 46084675 | 1019 | 6.00E−27 | 50.64 | 59 |
| | DMR_969 | chr11 | 77588647 | 77589194 | 547 | 6.80E−27 | 58.08 | 48 |
| | DMR_970 | chr6 | 168101223 | 168101754 | 531 | 6.80E−27 | 75.66 | 41 |
| * | DMR_971 | chr16 | 34802717 | 34803354 | 637 | 7.40E−27 | 44.27 | 49 |
| | DMR_973 | chr3 | 9600784 | 9601063 | 279 | 9.00E−27 | 69.95 | 42 |
| | DMR_974 | chr18 | 12912228 | 12912549 | 321 | 9.20E−27 | 50.77 | 46 |
| | DMR_975 | chr9 | 136345158 | 136345749 | 591 | 9.40E−27 | 68.62 | 46 |
| * | DMR_976 | chr10 | 1363251 | 1363487 | 236 | 1.00E−26 | 60.63 | 38 |
| | DMR_977 | chr19 | 56660613 | 56661040 | 427 | 1.00E−26 | 63.35 | 43 |
| | DMR_978 | chr7 | 38311319 | 38311553 | 234 | 1.00E−26 | 80.67 | 34 |
| | DMR_979 | chr2 | 10091277 | 10091877 | 600 | 1.20E−26 | 48.4 | 91 |
| | DMR_980 | chr2 | 91839916 | 91840363 | 447 | 1.20E−26 | 74.13 | 43 |
| | DMR_981 | chr4 | 1035426 | 1036169 | 743 | 1.20E−26 | 54.6 | 64 |
| * | DMR_983 | chr19 | 6233070 | 6234290 | 1220 | 1.30E−26 | 47.1 | 73 |
| | DMR_984 | chr22 | 39685915 | 39686067 | 152 | 1.30E−26 | 86.83 | 31 |
| * | DMR_985 | chr16 | 3413769 | 3414376 | 607 | 1.40E−26 | 51.93 | 56 |
| | DMR_986 | chr19 | 38386787 | 38387393 | 606 | 1.50E−26 | 42.08 | 97 |
| | DMR_987 | chr2 | 190627873 | 190628466 | 593 | 1.50E−26 | 74.23 | 40 |
| * | DMR_988 | chr5 | 141193877 | 141194285 | 408 | 1.50E−26 | 71.15 | 37 |
| | DMR_989 | chrX | 71492371 | 71492478 | 107 | 1.50E−26 | 76.79 | 33 |
| * | DMR_991 | chr8 | 37747765 | 37748396 | 631 | 1.60E−26 | 54.43 | 38 |
| * | DMR_992 | chr17 | 20902447 | 20902859 | 412 | 1.70E−26 | 70.07 | 39 |
| * | DMR_993 | chr17 | 886039 | 886757 | 718 | 2.00E−26 | 67.34 | 41 |
| * | DMR_994 | chr6 | 29015212 | 29015558 | 346 | 2.00E−26 | 42.97 | 51 |
| | DMR_995 | chr11 | 22432133 | 22433254 | 1121 | 2.20E−26 | 60.84 | 53 |
| | DMR_996 | chr9 | 127938096 | 127939084 | 988 | 2.20E−26 | 53.38 | 74 |
| * | DMR_997 | chr9 | 35792342 | 35793603 | 1261 | 2.70E−26 | 38 | 85 |
| | DMR_998 | chr1 | 161605885 | 161606620 | 735 | 2.80E−26 | 49.34 | 72 |
| * | DMR_999 | chr17 | 81121832 | 81122362 | 530 | 2.80E−26 | 51.88 | 72 |

Table I shows regions of the genome differentially-methylated in embryonic (pre-fetal) cells and cancers compared to their normal fetal of adult counterparts. Positions identified are from the Hg38 version of the human genome.

Methylation specific PCR (MSP) is the most commonly used method for detecting methylated or unmethylated DNA. MSP involves the step of bisulfite conversion. Sodium bisulfite is used to deaminate cytosine to uracil while leaving 5-methyl-cytosine intact. Methylation-specific PCR uses PCR primers targeting the bisulfite induced sequence changes to specifically amplify either methylated or unmethylated alleles. Bisulfite conversion destroys about 95% of the DNA. Since DNA concentrations are typically very low in the serum or plasma, a 95% reduction in DNA results in a detection rate of less than 50%.

Alternative methods use restriction enzymes that digest specifically either the methylated or unmethylated DNA. Enzymes that cut specifically methylated DNA are rare. However, enzymes that cut specifically unmethylated DNA are more readily available. Detection methods then establish whether digestion has occurred or not, and thus depending on the specificity of the enzyme used, allows detection of whether the underlying DNA was methylated or unmethylated and thus associated with cancer or not.

Methylation-sensitive enzyme digestion has been previously proposed. For example, Silva et al, British Journal of Cancer, 80:1262-1264, 1999 conducted methylation-sensitive enzyme digestion followed by PCR.

The present invention provides improved methods of methylation-sensitive detection of ctDNA utilizing novel differentially-methylated genes associated with the embryonic-fetal transition (EFT) and hence the embryo-onco phenotype thereby improving cancer diagnostic performance.

The method involves the use of a methylation-sensitive restriction enzyme to digest DNA sequences. DNA sequences of interest are selected which contain at least two restriction sites which may or may not be methylated. The method is preferably carried out with methylation-sensitive restriction enzymes which preferentially cleave unmethylated sequences compared to methylated sequences. Methylated sequences remain undigested and are detected. Digestion of unmethylated sequences at least one of the methylation-sensitive restriction enzyme sites results in the target sequence not being detected or amplifiable. Thus a methylated sequence can be distinguished from an unmethylated sequence. In one embodiment of the invention, the quantity of uncut target sequence detected in a biological sample, e.g. plasma or serum of cancer patients is higher than that demonstrated in a biological sample of the same type of healthy or cancer-free individuals since the target sequences are more highly methylated in cancer patients than healthy individuals.

In the alternative, restriction enzymes which cut methylated DNA can be used. Unmethylated DNA sequences are not digested and can be detected. In another embodiment of this invention, lower quantifies of the uncut DNA sequence are detected in a biological sample, e.g. plasma, or serum of cancer patients when compared with that demonstrated in a biological sample of the same type in cancer-free individuals.

In a preferred embodiment according to the present invention, the target sequence is detected by amplification by PCR. Real-time quantitative PCR can be used. Primer sequences are selected such that at least two methylation-sensitive restriction enzyme sites are present in the sequence to be amplified using such primers. The methods in accordance with the present invention do not use sodium bisulfite. Amplification by a suitable method, such as PCR, is used to detect uncut target sequence, and thus to identify the presence of methylated DNA which has not been cut by restriction enzymes.

In accordance with the present invention, any suitable methylation-sensitive restriction enzyme can be used. Examples of methylation-sensitive restriction enzymes that cut unmethylated DNA are listed in Table II.

TABLE II

| Name | Target Sequence | Effect of Methylation |
|---|---|---|
| AatII | GACGTC | blocked |
| AjiI | CACGTC | blocked |
| BstUI | CGCG | blocked |
| Bsh1236I | CGCG | blocked |
| Bsh12851 | CGRYCG | blocked |
| BshTI | ACCGGT | blocked |
| Bsp68I | TCGCGA | blocked |
| Bsp119I | TTCGAA | blocked |
| Bsp143II | RGCGCY | blocked |
| Bsul5I | ATCGAT | blocked |
| CseI | GACGC | blocked |
| Cfr10I | RCCGGY | blocked |
| Cfr42I | CCGCGG | blocked |
| CpoI | CGGWCCG | blocked |
| Eco47III | AGCGCT | blocked |
| Eco52I | CGGCCG | blocked |
| Eco72I | CACGTG | blocked |
| Eco105I | TACGTA | blocked |
| EheI | GGCGCC | blocked |
| Esp3I | CGTCTC | blocked |
| FspAI | RTGCGCAY | blocked |
| Hin6I | GCGC | blocked |
| Hin1I | GRCGYC | blocked |
| HpaII | CCGG | blocked |
| Kpn2I | TCCGGA | blocked |
| MluI | ACGCGT | blocked |
| NotI | GCGGCCGC | blocked |
| NsbI | TGCGCA | blocked |
| PauI | GCGCGC | blocked |
| PdiI | GCCGGC | blocked |
| P112311 | CGTACG | blocked |
| Pfl23II | CGTACG | blocked |
| Ppu21I | YACGTR | blocked |
| Psp1406I | AACGTT | blocked |
| PvuI | CGATCG | blocked |
| SalI | GTCGAC | blocked |
| SgsI | GGCGCGCC | blocked |
| SmaI | CCCGGG | blocked |
| SmuI | CCCGC | blocked |
| SsiI | CCGC | blocked |
| TaiI | ACGT | blocked |
| TauI | GCSGC | blocked |

Table II shows examples of methylation-sensitive restriction enzymes. The letter codes in the recognition sequences represent different combinations of nucleotides and are summarized as follows: R=G or A; Y=C or T; W=A or T; M=A or C; K=G or T; S=C or G; H=A, C or T; V=A, C or G; B=C, G or T; D=A, G or T; N=G, A, T or C. The CpG dinucleotide(s) in each recognition sequence is/are underlined. The cytosine residues of these CpG dinucleotides are subjected to methylation. *The methylation of the cytosine of the CpG dinucleotides in the recognition sequence would prevent enzyme cutting of the target sequence.

The target sequence includes two or more methylation-sensitive restriction enzyme sites. Such sites may be recognized by the same or different enzymes. However, the sites are selected so that at least two sites in each sequence are digested when unmethylated when using enzymes which preferentially cleave unmethylated sequences compared to methylated sequences.

In a less preferred embodiment the target sequence contains at least two sites which are cut or cleaved by restriction enzymes which preferentially cleave methylated sequences. The two or more sites may be cleaved by the same or different enzymes.

Any DMR listed in Table I may be used in accordance with the present invention. Preferred DMR regions are those that contain at least two methylation-sensitive restriction enzyme sites. Generally such methylation markers are genes where promoter and/or encoding sequences are methylated in embryonic cells and cancer patients. Preferably the selected sequences are not methylated or are methylated to a lesser extent in fetal and adult cells and non-cancer or cancer-free individuals.

Thus, in accordance with an alternative aspect of the present invention, there is provided a method for the detection or monitoring of cancer using a biological sample selected from blood, plasma, serum, saliva, urine from an individual, said method comprising:
(a) obtaining DNA from the said biological sample;
(b) digesting the DNA sample with one or more methylation-sensitive restriction enzymes;
(c) quantifying or detecting a DNA sequence of interest after step (b) wherein the DNA sequence is a sequence listed in Table I; and
(d) comparing the level of the DNA sequence from the individual to a normal standard, to detect, prognosticate or monitor cancer.

In accordance with the method of the present invention a sample is taken or obtained from the patient. Suitable samples include blood, plasma, serum, saliva and urine. Samples to be used in accordance with the present invention include whole blood, plasma or serum. Methods for preparing serum or plasma from whole blood are well known among those of skill in the art. For example, blood can be placed in a tube containing EDTA or a specialized commercial product such as Vacutainer SST (Becton Dickenson, Franklin Lake, N.J.) to prevent blood clotting, and plasma can then be obtained from whole blood through centrifugation. Serum may be obtained with or without centrifugation following blood clotting. If centrifugation is used then it is typically, though not exclusively conducted at an appropriate speed, for example, 1500-3000×g. Plasma or serum may be subjected to additional centrifugation steps before being transferred to a fresh tube for DNA extraction.

Preferably, DNA is extracted from the sample using a suitable DNA extraction technique. Extraction of DNA is a matter of routine for one of skill in the art. There are numerous known methods for extracting DNA from a biological sample including blood. General methods of DNA preparation, for example described by Sambrook and Russell, Molecular Cloning a Laboratory Manual, 3^{rd} Edition (2001) can be followed. Various commercially available reagents or kits may also be used to obtain DNA from a blood sample.

In accordance with the invention, the DNA containing sample is incubated with one or more restriction enzyme(s) which preferentially cut unmethylated DNA under conditions such that where two or more restriction enzyme sites are present in the target sequence in the unmethylated state, the restriction enzyme(s) can cut the target sequence at least one such site. In accordance with an alternative aspect of the invention, a DNA sample is incubated with one or more restriction enzymes which only cut methylated DNA under conditions such that where two or more restriction enzyme sites are present in the methylated state, the restriction enzyme(s) can cut the target sequence at least one such site.

Preferably samples are incubated under conditions to allow complete digestion. This may be achieved, for example by increasing the incubation times and/or increasing the quantity of the enzyme used. Typically, the sample will be incubated with 100 active units of methylation-sensitive restriction enzyme for a period of up to 16 hours. It is a matter of routine for one of skill in the art to establish suitable conditions based on the quantity of enzyme used.

After incubation, uncut target sequences are detected. Preferably, these sequences are detected by amplification, for example using the polymerase chain reaction (PCR).

DNA primers are designed to amplify a sequence containing at least two methylation-sensitive restriction enzyme sites. Such sequences can be identified by looking at DNA methylation markers and identifying restriction enzyme sites within those markets which are recognised by methylation-sensitive enzymes. For example using the recognition sequences for the methylation-sensitive enzymes identified in Table II, suitable target sequences can be identified in Table I.

When using methylation-sensitive enzymes, altered quantities of the target sequence will be detected depending on the methylation status of the target sequence in a particular individual. In the preferred aspect of the present invention using methylation-sensitive restriction enzymes which preferentially cut unmethylated DNA, the target sequence will not be detected in the unmethylated state, for example in a healthy individual. However, where the target sequence is methylated, for example in a selected sample from a cancer patient, the target sequence is not cut by the restriction enzyme and the target sequence can thus be detected by PCR.

Thus, the method can be used to determine the methylation status of the target sequence and provide an indication of the cancer status of the individual.

The methods of the present invention may additionally include quantifying or detecting a control sequence. The control sequence is selected which does not show aberrant methylation patterns in cancer. In accordance with a preferred aspect of the present invention, the control sequence is selected to contain at least two methylation-sensitive restriction enzyme recognition sites. Preferably, the control sequence is selected to contain the same number of methylation-sensitive restriction enzyme recognition sites as the DNA sequence of interest. Typically the presence or absence of such control sequences is detected by amplification by the polymerase chain reaction after digestion with the methylation-sensitive restriction enzyme(s). Such control sequences can be used to assess the extent of digestion with the one or more methylation-sensitive restriction enzymes. For example, if after digestion with the methylation-sensitive restriction enzyme(s) control sequences are detectable, this would indicate that the digestion is not complete and the methods can be repeated to ensure that complete digestion has occurred. Preferably the control sequence is selected to contain the same methylation-sensitive restriction enzyme sites that are present in the target sequence.

The present methods can be used to assess the tumor status of an individual. The methods can be used, for example, in the diagnosis and/or prognosis of cancer. The methods can also be used to monitor the progress of cancer, for example, during treatment. The methods can also be used to monitor changes in the levels of methylation over time, for example to assess the susceptibility of an individual to cancer, and the progression of the disease. The methods can also be used to predict the outcome of disease or the likelihood of success of treatment.

### Primer Design

In another aspect of the invention, there is provided probes and primers for use in the method of the invention. Firstly, there is provided a set of primers or a detectably-labelled probe for the detection or monitoring of cancer in a biological sample selected from blood, plasma, serum, saliva, urine from an individual. The set of primers comprises or consists of primers designed using guidelines known in the art (Davidovic R S et al, 2014. Methylation-specific PCR: Four steps in primer design. Cent. Eur. J. Biol. 9:1127-1139) incorporated herein by reference. One experienced in the art will recognize that numerous function forward and reverse primers for PCR can be generated to the DMRs of Table I, some of which may include sequences up to 300 bp 5′ or 3′ of the DMR regions described herein. Online resources are available to teach methods of primer design. Examples of such resources include MSPprimer (http://www.mspprimer.org/cgi-bin/design.cgi), MethMarker (http://methmarker.mpi-inf.mpg.de/), Beacon designer (http://www.premierbiosoft.com/molecular_beacons/), and Primo MSP (http://www.changbioscience.com/primo/primom.html). In general, the steps of primer selection will facilitate the differential PCR amplification of methylated and unmethylated cytosine residues and will include: 1) downloading the DMR sequence from online resources, 2) identifying regions rich in CpG sites, 3) primers with at least one CpG site at its 3′ end, 4) a greater number of non-CpG cytosines is preferred, 5) primer lengths are generally 20-30 nucleotides in length, and 6) because BIS treatment fragments DNA, reaction products should selected to be less than 300 bp in length. In silico analysis of primer designs can also be tested using resources such as those available on the UC Santa Cruz Genome Browser (https://genome.ucsc.edu/cgi-bin/hgPcr?hgsid=748426759_0fMTAb4eddROJREtR7blyFe6YmpG).

The probes are detectably-labelled. The detectable label allows the presence or absence of the hybridization product formed by specific hybridization between the probe and the target sequence to be determined. Any label can be used. Suitable labels include, but are not limited to, fluorescent molecules, radioisotopes, e.g. ¹²⁵I, ³⁵S, enzymes, antibodies and linkers such as biotin.

Methods for induced tissue regenerated (“iTR”) may also be used with the present invention. Examples of such methods are disclosed in International Patent Application PCT/US2019/028816, titled “Improved Methods for Inducing Tissue Regeneration and Senolysis in Mammalian Cells,” incorporated herein by reference in its entirety, International Patent Application Publication WO 2014/197421, titled “Compositions and Methods for Induced Tissue Regeneration in Mammalian Species,” incorporated herein by reference in its entirety, and WO/2017/2142A1, titled “Improved Methods for Detecting and Modulating the Embryonic-Fetal Transition in Mammalian Species,” incorporated herein by reference in its entirety.

Methods for induced cancer maturation (“iCM”) may also be used with the present invention. Examples of such methods are disclosed in WO/2017/2142A1, titled “Improved Methods for Detecting and Modulating the Embryonic-Fetal Transition in Mammalian Species,” incorporated herein by reference in its entirety.

In another aspect, there is provided kits for use in the method of invention. Firstly, there is provided a kit for the detection or monitoring of cancer in a biological sample selected from blood, plasma, serum, saliva, urine from an individual. The kit comprises primers designed to detect methylated CpGs in the DMRs of Table I.

Secondly, there is provided a kit for use as a control during the detection or monitoring of cancer in a biological sample selected from blood, plasma, serum, saliva, urine from an individual. The kit comprises primers designed to detect methylated CpGs in the DMRs of Table I.

The kits of the invention may additionally comprise one or more other reagents or instruments which enable the method of the invention as described above to be carried out. Such reagents or instruments include one or more of the following: suitable buffer(s) (aqueous solutions), PCR reagents, fluorescent markers and/or reagents, means to obtain a sample from individual subject (such as a vessel or an instrument comprising a needle) or a support comprising wells on which reactions can be done. Reagents may be present in the kit in a dry state such that the fluid sample resuspends the reagents. The kit may, optionally, comprise instructions to enable the kit to be used in the method of the invention.

The invention is hereinafter described in more detail by reference to the Examples below.

### EXAMPLES

### Example 1. The Use of Markers of the Embryo-Onco Phenotype to Characterize Malignant Cells

As disclosed in the present invention, there are many cell type-specific DNA methylation marks as a result of the different patterns of gene expression in diverse differentiated cells. Therefore, the validation of true DMRs useful in detecting or diagnosing the embryonic vs fetal or embryonic vs adult phenotypes of cells requires a comparison of embryonic, but nevertheless differentiated cells with post-EFT cells, such as adult differentiated cells of the same differentiated type. And to determine whether those DMRs are pre or post-EFT in nature, it is necessary to also observe DMRs from malignant cells from the corresponding differentiated cell type. In this example, we compare of embryonic, adult, and malignant osteochondral mesenchyme; or embryonic, adult, and malignant skeletal myoblasts, or embryonic, adult, and malignant preadipocytes and embryonic, adult, and malignant skeletal muscle myoblasts.

By way of nonlimiting example of the present invention, DMR_327 with the position of chr10:89837217-89837885 in the + strand of hg38 has the following sequence with CpG sites capitalized and underlined and an example of a methylation-specific restriction endonuclease site, in this case for the restriction endonuclease SmaI at nucleotide positions 53 and 86 (enclosed in box below):

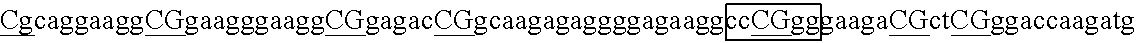

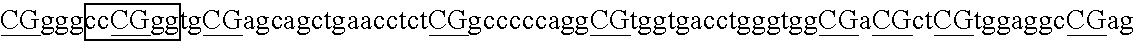

gagcttctggacCGgCGcttCGggggaccaagtggagaggctgctggagttgtCGcctgagtcctcccttagttcttCGCGcCG
gcctCGcccaCGgctcCGggtcccagcCGccactgcagtctcCGcagcacCGagCGgggctccacCGactCGCGacct
ccagcctcCGcctcaagggcagggagCGCGgctgggtctctggaaagccatttttaaatcactgcctctgctgcccccatgtgaggtC
GgagtgtcctccccCGtctttgctttcaggttctttcaggttcctttgggcaaaccCGcagctaagagtccagcttgtgaacttgaacctgaa
cttgctgaagaagctccCGgCGgccccctgctgtctgCGgcctttgtttgagggagaggctggggtcaccCGgttgggcCGcattt
cCGgggcCGtcacctgtCGggctgccaggcCGCGCGtaccttgtcccatCGggggctctgctctgccccctgCGctgatga
CGc.

Additional methylation-specific restriction sites are those for Cfr10I at nucleotide positions 29, 167, and 237 and TauI at nucleotide positions 269, 349, 522, 539, 580, 617 of the above DMR. Therefore, the choice of primers 5′ and 3′ of the said methylation-specific restriction sites, will yield a greater percentage of full-length reaction product in adult cells compared to cells with an embryonic epigenetic profile or cancer cells that have reverted to an embryo-onco epigenetic profile. By way of nonlimiting example, the choice of a forward primer 5′-aggcggagaccggcaagag-3′ and a reverse primer 5′-agaactaagggaggactcaggc-3′ will yield a 212 bp reaction product in normal adult cells and no reaction product in the case of embryonic or cancer cell-derived DNA pre-treated with SmaI endonuclease.

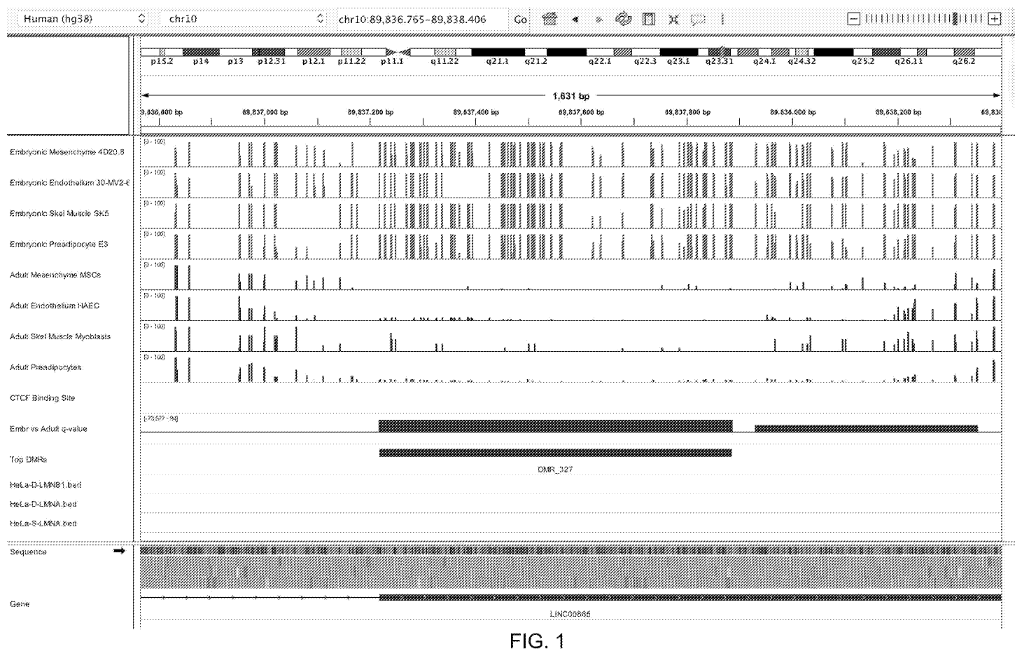

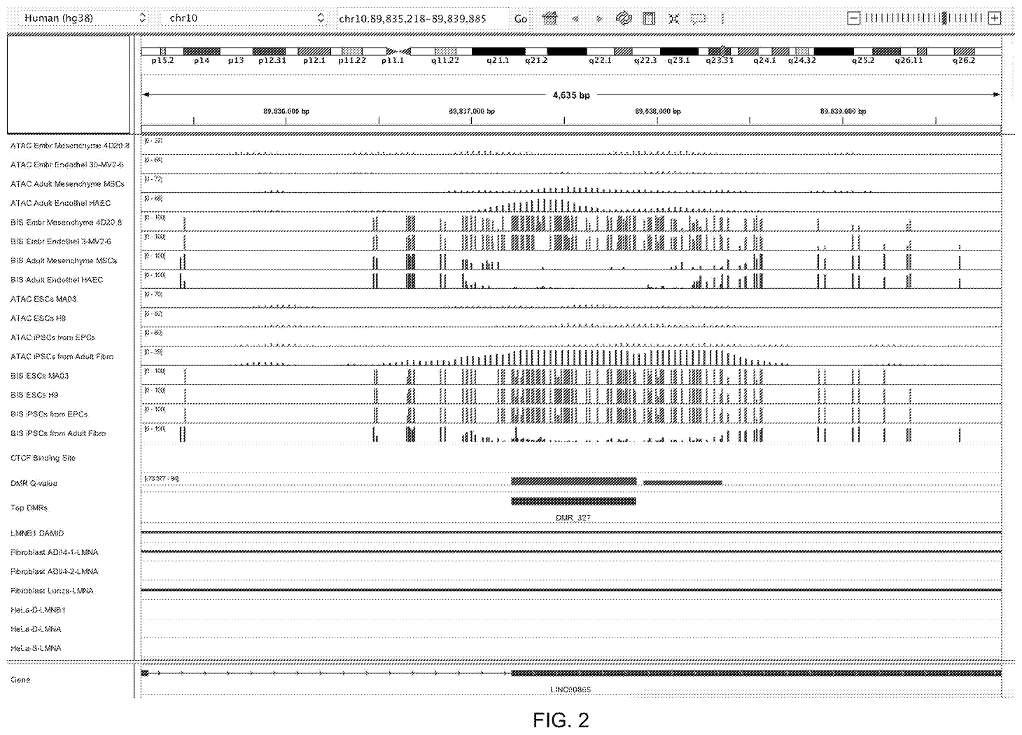

As shown in FIG. 1, all 53 of 53 possible CpG sites within the DMR were hypermethylated in embryonic progenitors compared to their adult counterparts. As shown in FIG. 2, The methylation marks also showed the embryonic pattern in two hES cell line (H9 and MA03), as well as an iPS cell line designated EH3 generated from a line of clonal EPCs designated EN13 (Vaziri et al 2010, Spontaneous reversal of the developmental aging of normal human cells following transcriptional reprogramming Regen Med 5(3):345-363), however iPS cells produced from adult human dermal fibroblasts retained an adult methylation pattern despite the fact that the iPSC cell line exhibiting a pluripotent pattern of gene expression.

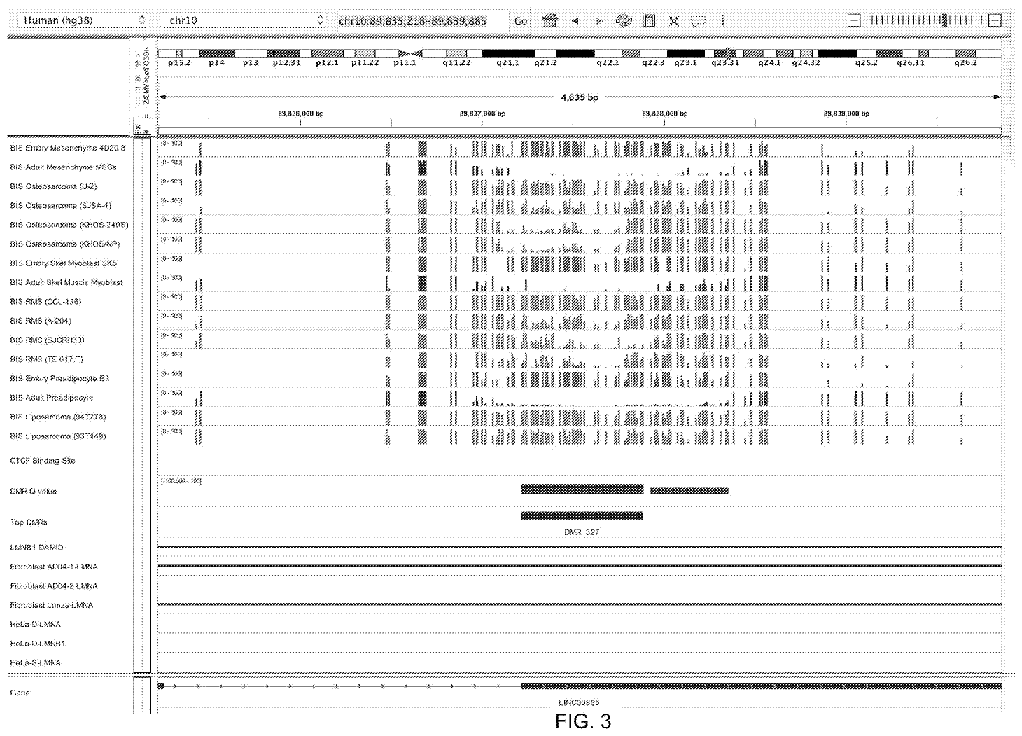

As shown in the IGV image of FIG. 3, CpG methylation results obtained by BIS-seq of a hES cell-derived clonal embryonic progenitor cell line was markedly higher in DMR_327 than corresponding methylation of the normal adult counterpart being bone marrow mesenchymal stem cells (4D20.8 and MSCs respectively). The corresponding cancer cell lines derived from osteogenic mesenchyme (the osteosarcoma cell lines U-2, SJSA-1, KHOS-240S, and KHOS/NP) showed a significant correlation with embryonic cells as opposed to their adult counterparts. Also shown are comparable CpG methylation results obtained by BIS of a hES cell-derived clonal embryonic progenitor cell line corresponding to skeletal myoblasts and an adult derived normal counterpart being adult skeletal myoblasts (SK5 and Adult Skel Muscle Myoblasts respectively) followed by corresponding adult-derived cancer cell lines derived from muscle mesenchyme (the rhabdomyosarcoma (RMS) cell lines CCL-136, A-204, SJCRH30, and TE 617.T). Also shown are CpG methylation results obtained by BIS of a hES cell-derived clonal embryonic progenitor cell line corresponding to white adipocyte progenitors (E3) and an adult derived normal counterpart being adult preadipocytes and the lipogenic cancer cell lines (94T778 and 93T449). In this example, the embryonic methylation pattern in DMR_327 predicts with 100% accuracy the malignant status of 10 different cancer cell lines compared to normal counterparts.

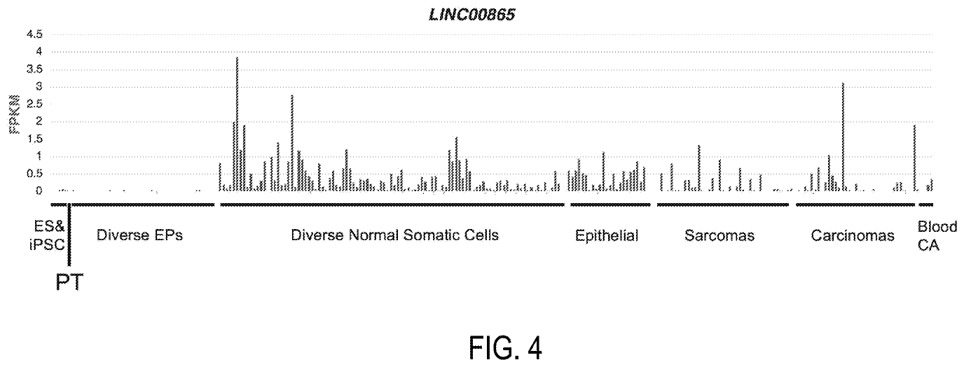

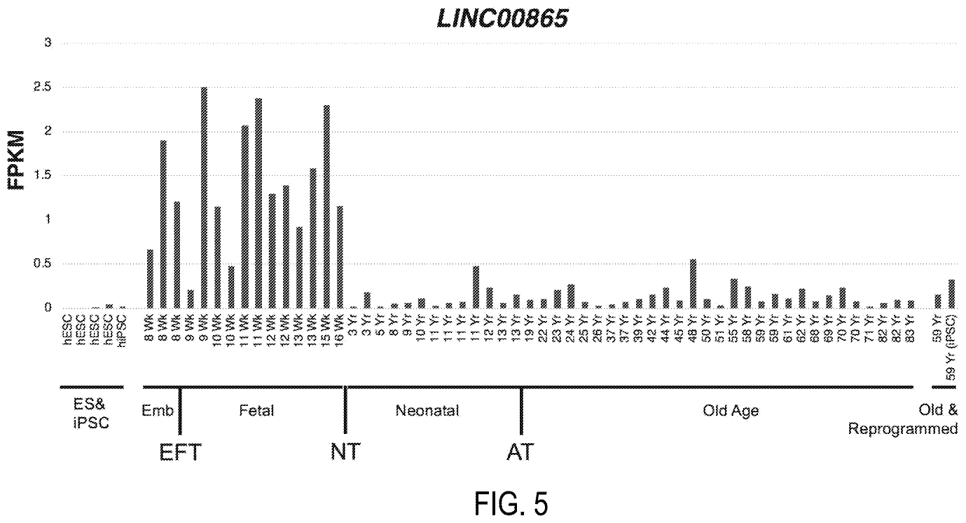

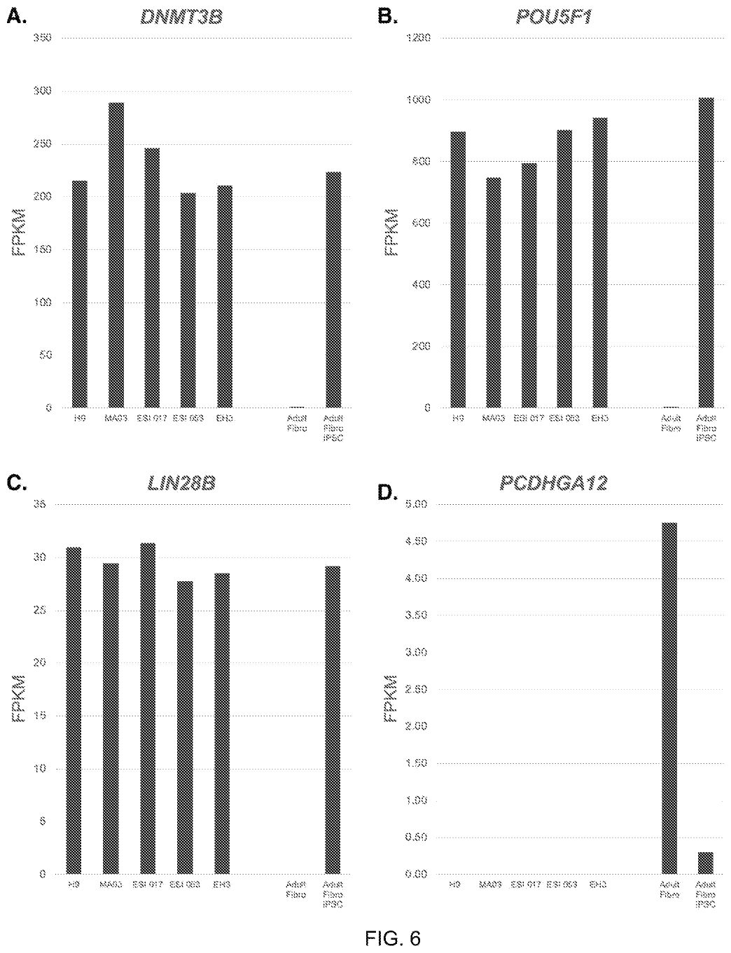

As shown in FIG. 4, a transcript designated LINC00865 coinciding with DMR_327 is expressed at low to undetectable levels in hES cells and iPS cells derived from diverse clonal embryonic progenitor lines (labelled ES & iPSC in FIG. 4), but is expressed in most fetal and adult-derived diverse somatic cell types. As shown in FIG. 5, the transcript is already expressed in cultured late embryonic stages of skin fibroblasts (8 weeks gestation), and is not restored to the low to undetectable levels of expression of hES cells in adult skin-derived iPS cells, even though the adult skin-derived iPS cells were abundantly expressing other markers of hES cells such as OCT4, NANOG, LIN28A (shown in FIG. 6), SOX2, as well as other hES cell markers. Also shown in FIG. 6 is the incomplete reprogramming of the expression of PCDHGA12, previously disclosed as an fetal/adult-onset marker (“Improved Methods for Detecting and Modulating the Embryonic-Fetal Transition in Mammalian Species” (international patent application publication number WO 2017/214342, incorporated herein by reference in its entirety). This demonstrates the utility of the DMRs described herein as markers more sensitive than traditional markers of pluripotency of the complete epigenetic reprogramming of fetal or adult-derived somatic cells.

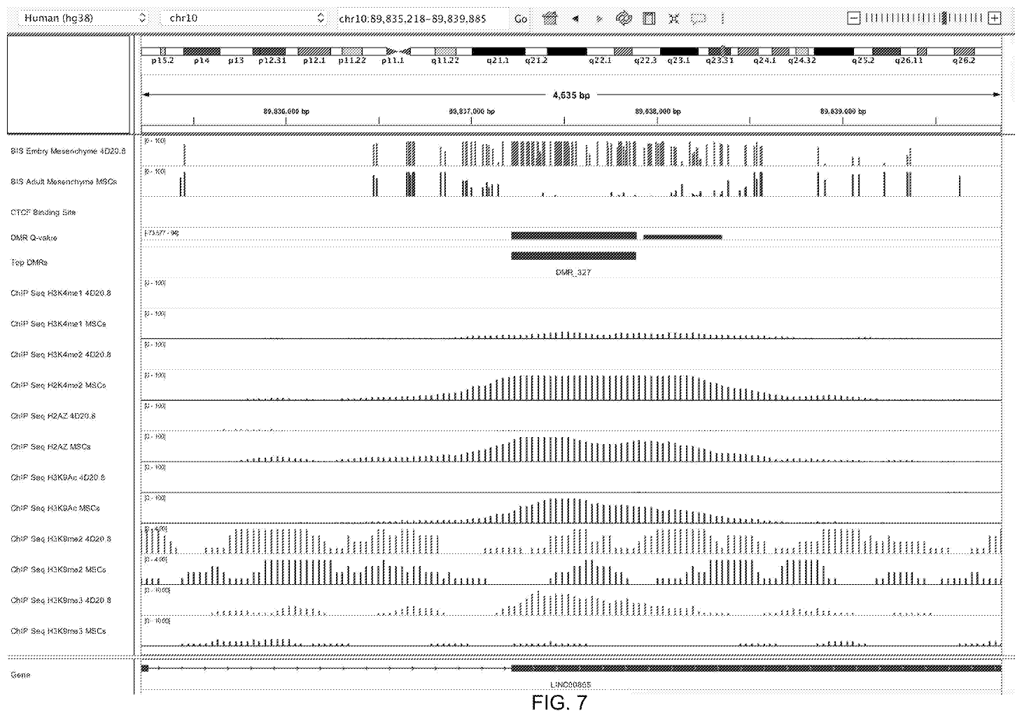

Since the hypermethylation of DMR_327 also predicted normal and cancer cells lines that were in the pre-EFT state and which did not express LINC00865, this example demonstrates the usefulness of the DMRs of the present invention in determining the maturation state of said cells. As shown in FIG. 7, antibody to the post-translation modification of histone H3 (H3K4me1 and H3K4me2), H2AZ, and H3K9Ac precipitates chromatin with the differential de-methylation of the DMRs of the present invention and is therefore useful in enriching post-EFT DMRs of the present invention or alternatively, removing post-EFT DMRs when attempting to detect pre-EFT DMRs. As also shown in FIG. 7, antibody to H3K9me2 and H3K9me3 precipitates pre-EFT DMRs of the present invention, and is therefore useful in enriching pre-EFT DMRs of the present invention.

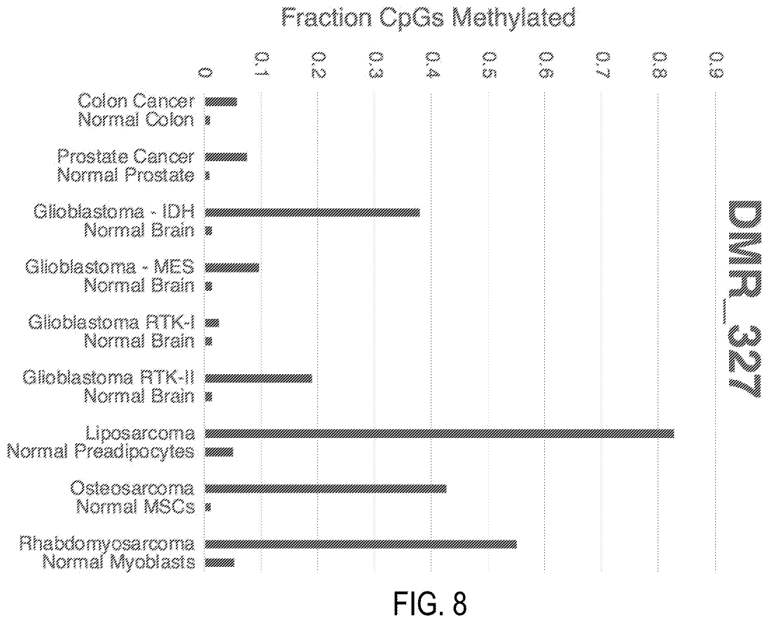

As shown in FIG. 8, the DMRs of the present invention apply to all cancer types (are pan-cancer). As shown, by way of nonlimiting example, DMR_327 is significantly hypermethylated in carcinomas such as in colon cancers compared to normal colon, prostate cancer when compared to normal prostate, brain cancers such as diverse glioblastomas compared to normal brain, and sarcoma cells compared normal counterparts including by way of nonlimiting example, liposarcomas, osteosarcomas, and rhabdomyosarcomas.

As described herein one skilled in the art would know that additional, although less common methylated CpG marks can be found within bp 5′ or 3′ of the DMR. In this example, within the 500 bp expanded range of DMR_327, a total of 94 or an additional 41 CpG sites many of which show increased methylation in embryonic vs adult cells and are also hypermethylated in corresponding cancer types.

### Example 2. The Induction of Cancer Maturation (iCM) in Dematured (DC) Cells

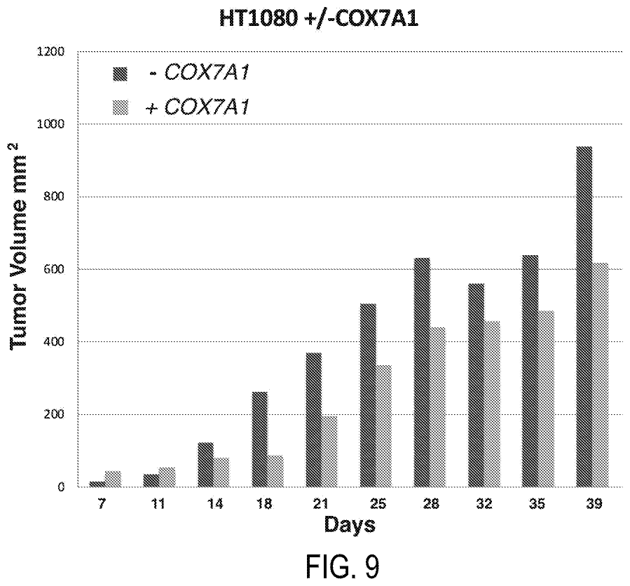

In this example we induce cancer maturation in a cancer cell line displaying DMR markers of the present invention of a pre-fetal state such as hypermethylation of DMR_038 co-localizing with the gene COX7A1 which is not expressed in most pre-fetal differentiated cell types, is progressively increased in expression during fetal and adult development, and repressed in cancer cells displaying a pre-fetal (DC) phenotype. As an example of induced cancer maturation, we expressed the COX7A1 gene at adult levels in the DC fibrosarcoma cell line HT1080. We then analyzed the take rate and growth kinetics of the HT1080 cells with (HT1080+COX7A1) and without (HT1080-COX7A1) COX7A1 introduced by means of lentiviral infection. Growth kinetics of the line with and without iCM was then measured in female athymic nude mice. Ten mice were injected with 5×10⁶ cells subcutaneously once per day of either the native HT1080 cells or the HT1080 cells exogenously expressing COX7A1 (iCM treated). All animals were subjected to a complete necropsy including examination of the carcass and musculoskeletal system; all external surfaces and orifices, cranial cavity and external surfaces of the brain; and thoracic, abdominal and pelvic cavities with their associated organs and tissues. When masses were present in the right flank region, they were carefully removed and the subcutaneous and surrounding tissues were examined for any signs of metastasis from the primary mass. The study pathologist conducted all necropsies and performed evaluation of all of the tissues for the presence of primary and metastatic tumors. Gross findings were limited to masses on the right flank. As shown in FIG. 9, iCM treatment of the DC cells significantly slowed the growth of the resulting tumors.

### Example 3. Increased Sensitivity of Cells with a Post-EFT Pattern of Gene Expression to Senolysis when Treated with iTR Agents

The present invention describes the use of DMR markers of the EFT to determine the sensitivity of cells to undergo apoptosis in the presence of chemotherapeutic or radiotherapy agents that damage DNA or otherwise induce apoptosis. Since the selective removal of cells with DNA damage includes cells commonly designated as “senescent” cells, such as those with significant loss of telomeric DNA, we choose to designate the purposeful induction of apoptosis in damaged cells as “senolysis” as an inclusive term for the induction of apoptosis in cancer cells by the chemotherapeutic and radiotherapies described herein, as well as cells that have significant DNA damage from intrinsic sources such as with telomeric attrition.

The pre-EFT (DC) fibrosarcoma cell line HT1080 was infected with lentivirus expressing COX7A1 together with a control line expressing green fluorescent protein (GFP). The resulting cells were treated with 0, 0.37, and 37 uM camptothecin to generate a DNA damage response and apoptosis. TUNEL (TdT-mediated dUTP-X nick end labeling) adds label to termini of ssDNA and dsDNA. Readout used was fluorescent nuclei read by microscopy. In brief, cell lines were cultured in 96-well plates at 5000 cells/well and grown over night. The following day, the growth medium was removed and replaced with growth medium containing compounds and controls. After 24 h, the cells are fixed for 20 minutes using 4% PFA. Plates are stored at 4 deg C. in PBS until processing. Fixed cells were permeabilized with 0.1% Triton X-100 and 0.1% sodium citrate for 2 minutes on ice. Cells were washed 3 times and incubated in TUNEL reaction buffer according to manufactures protocol for 60 minutes at 37 deg C. Samples were washed 3 times in PBS and stored in 100 uls of PBS for imaging. Cells are stained with Hoechst stain for 10 minutes at RT and washed 1 time with PBS. Each well was imaged using 5× objective. 9 images per well were collected and analyzed for total cell number and number of cells stained for apoptosis.

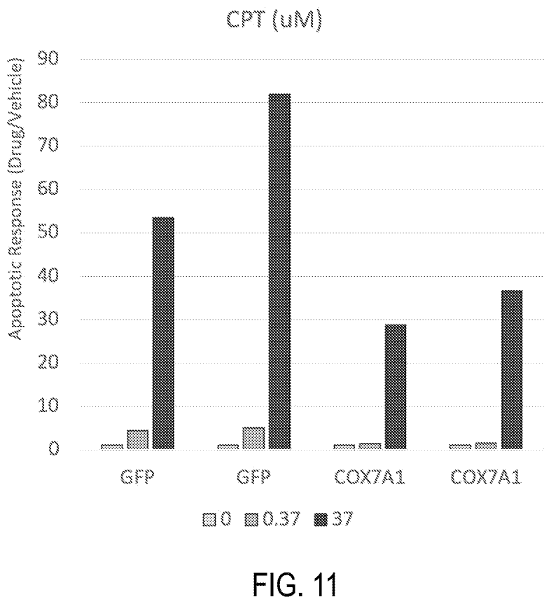

The expression of COX7A1 in the HT1080 fibrosarcoma cell line is associated with significantly decreased sensitivity to apoptosis as shown in FIG. 11 (p<0.05). Similarly, normal pre-EFT vascular progenitors were more sensitive to apoptosis at 37 uM of camptothecin (39% apoptosis) compared to adult aortic endothelial counterparts (25.5% apoptosis). In addition, H₂O₂-mediated apoptosis was measured in post-EFT cells (adult bone marrow MSCs) before and after knock-down of COX7A1 expression. While 82.4% of normal MSCs survived 300 uM of H₂02, the knockdown of COX7A1 as an iTR modality resulted in 59.8% of the MSCs surviving (p<0.05), that is, iTR resulted in increased sensitivity to senolysis. Lastly, PCDHB2 positive exosomes derived from the pre-EFT clonal embryonic vascular endothelial line 30-MV2-6 induced senolysis specifically in senescent fibroblasts. Each of these examples demonstrate the relative resistance of post-EFT cells to chemotherapy or radiotherapy induced senolysis, the modification of that resistance to senolysis by iTR and iCM factors, and the value of the DMRs of the present invention in determining the maturation status of cells, in particular, whether they display a pre- or post-EFT status.

### Example 4. The Mature Post-EFT (AC) Phenotype as Opposed to Relatively Undifferentiated “Cancer Stem Cells” Correlates with Cells Surviving Chemotherapy and Radiation Therapy

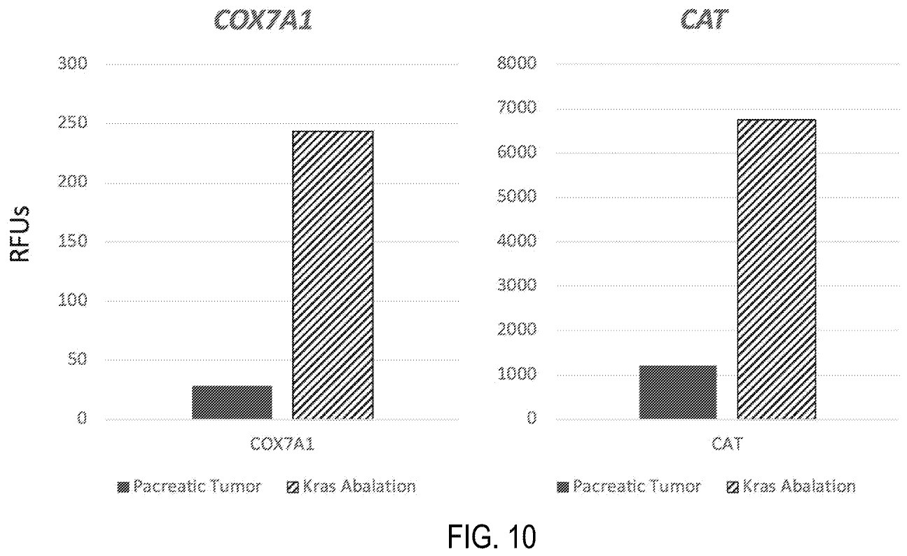

The current widely accepted model of cancer stem cells (CSCs) posits that CSCs are relatively undifferentiated cells that like hematopoietic stem cells divide relatively rarely and hence survive many chemotherapeutic or radiotherapy protocols and repopulate the body after the therapy. The present invention instead teaches the contrary, that these surviving CSCs are instead cancer cells that display a post-EFT pattern (i.e. a more mature pattern) of gene expression. Using the expression of the gene COX7A1 as a transcriptional marker of pre- or post-EFT cells wherein COX7A1 is expressed in post-EFT cells, we observe that pancreatic cancer with ablated KRAS leading to pancreatic CSCs, results in increased COX7A1 and CAT (also a post-EFT marker), not decreased as predicted by current models (FIG. 10). In addition, the treatment of xenograft breast tumors derived from the breast cancer cell line MCF-7 with the anti-tumor anthracycline antibiotic doxorubicin results in COX7A1 RFU levels of 5.04 and 5.57 compared to controls of 3.84 and 4.52 in controls, again indicative of the more mature state of surviving cells following chemotherapy. In addition, in the example of rectal cancer, levels of COX7A1 expression were 92.7 RFUs following radiotherapy compared to 75 RFUs in untreated rectal cancer (p<0.05). Using the pre-EFT marker CPT1B wherein CPT1B is expressed in pre-EFT cells and in cancer cells (corresponding to DMR_087), platinum-resistant ovarian cancer cells expressed on average 384.9 RFU of CPT1B while cis-platin sensitive ovarian cancer expressed a mean expression of 719.5 RFU (p<0.05), consistent with chemotherapy sensitivity corresponding to higher CPT1B expression, higher methylation of DMR_087, and a pre-EFT (DC) phenotype. Lastly, vincristine-sensitive ovarian cancer cells lines expressed on average 75.2 RFU of CPT1B while vincristine-resistant ovarian cancer cells expressed a mean expression of 43.9 RFU (p<0.05), consistent with chemotherapy sensitivity corresponding to higher CPT1B expression, higher methylation of DMR_087, and a pre-EFT (DC) phenotype.

1. A method to determine the developmental staging of cells that were the source of a sample of human DNA comprised of the steps: 1) identifying DMRs differentially-methylated in embryonic (pre-fetal) cells compared to their fetal (prenatal) or adult counterparts, 2) determining whether a sample of human DNA contains methylated or unmethylated CpG epigenetic marks within said DMRs, 2) use of said markers for the diagnosis, prognosis and/or treatment of cancer.

2. The method of claim 1, wherein the use of the information relating to the methylated or unmethylated CpG epigenetic marks is directed to determining the completeness of the in vitro transcriptional reprogramming of cells to pluripotency (iPS cell reprogramming) or the in vivo reprogramming of cells and tissues to reverse aging or to induce tissue regeneration (iTR) in diverse tissues in the body.

3. The method of claim 1, wherein said embryonic (pre-fetal), fetal (prenatal), or postnatal (adult) cells are human.

4. The method of claim 1, wherein said methylated or unmethylated CpG epigenetic marks are identified from blood-derived cfDNA.

5. The method of claim 1, wherein said methylated or unmethylated CpG epigenetic marks are identified from blood-derived cfDNA through the use of methylation-specific restriction endonuclease digestion.

6. The method of claim 1, wherein said methylated or unmethylated CpG epigenetic marks are identified from blood-derived cfDNA through the use of methylation-specific PCR primers.

7. The method of determining the optimum therapeutic strategy for a given cancer in humans comprised of the steps: 1) obtaining DNA from the tumor, 2) determining the relative methylation of one or more of the DMRs of the present invention in said DNA sample, 3) determining whether said DMRs are statistically correlated with pre- or post-EFT, 4) treating pre-EFT cells with chemotherapy, iCM, or radiation therapy, 5) treating post-EFT cells as CSCs and treating with iTR.
